(19) United States
(12) Patent Application Publication
(10) Pub. No.: US 2024/0071622 A1
(43) Pub. Date: Feb. 29, 2024

(54) CLINICAL CLASSIFIERS AND GENOMIC CLASSIFIERS AND USES THEREOF

(71) Applicant: Veracyte, Inc., South San Francisco, CA (US)

(72) Inventors: Giulia C. Kennedy, San Francisco, CA (US); Yoonha Choi, South San Francisco, CA (US); Joshua Babiarz, South San Francisco, CA (US); Jianghan Qu, South San Francisco, CA (US); Daniel Pankratz, South San Francisco, CA (US); Shuyang Wu, South San Francisco, CA (US); Jie Ding, South San Francisco, CA (US); Sangeeta Bhorade, South San Francisco, CA (US); Lori Lofaro, South San Francisco, CA (US); P. Sean Walsh, South San Francisco, CA (US); Jing Huang, South San Francisco, CA (US)

(21) Appl. No.: 18/328,541

(22) Filed: Jun. 2, 2023

Related U.S. Application Data
(63) Continuation of application No. PCT/US21/61649, filed on Dec. 2, 2021.
(60) Provisional application No. 63/121,153, filed on Dec. 3, 2020.

(51) Int. Cl. G16H 50/30; G16B 20/20; G16B 25/10; G16B 40/20

(52) U.S. Cl. CPC G16H 50/30; G16B 20/20; G16B 25/10; G16B 40/20

(57) ABSTRACT

Provided herein are methods and systems for analyzing a sample of a subject to determine whether the subject has, or is at risk of having or developing, a cancer, such as lung cancer.

### CROSS REFERENCE

This application is a continuation application of International Application No. PCT/US2021/061649, filed Dec. 2, 2021, which claims the benefit of U.S. Provisional Application No. 63/121,153, filed Dec. 3, 2020, each of which is entirely incorporated herein by reference.

### BACKGROUND

Lung cancer is the deadliest form of cancer in the United States and the world. An estimated 221,000 new lung cancer diagnoses are expected in the United States in 2015, and approximately 158,000 men and women are expected to fall victim to the disease during the same time period. The high mortality rate is due, in part, to a failure in 70% of patients to detect lung cancer when it is localized and surgical resection remains feasible. Additionally, diagnosis procedures for lung cancer are often painful and invasive.

### SUMMARY

Disclosed herein is a method, comprising, upon obtaining a first level of risk of malignancy of a subject for having or developing a cancer, obtaining a data set corresponding to a sample of the subject; in a programmed computer, using a classifier to assign the data set corresponding to the sample a second level of risk of malignancy for having or developing the cancer; and electronically outputting a report comprising the second level of risk of malignancy assigned to the sample of the subject, wherein the second level of risk of malignancy is determined with a negative predictive value greater than 90%. The first level of risk of malignancy and the second level of risk of malignancy can be different. The second level of risk of malignancy can be greater than the first level of risk of malignancy.

The second level of risk of malignancy can be less than the first level of risk of malignancy. The first level of risk of malignancy can be less than 10% and the second level of risk of malignancy can be less than 1%. The first level of risk of malignancy can be 10% to 60% and the second level of risk of malignancy can be greater than 60%. The first level of risk of malignancy can be 10% to 60% and the second level of risk of malignancy can be less than 10%. The first level of risk of malignancy can be greater than 60% and the second level of risk of malignancy greater than 90%.

The subject can have or can be suspected of having a nodule. The nodule can be identified by imaging analysis. The nodule can be identified as having the first level of risk of malignancy of greater than 60% for lung cancer. The nodule can be identified as having the first level of risk of malignancy of less than 10% for lung cancer. The imaging analysis can be low-dose computed tomography (LDCT), computer aided tomography (CAT), or magnetic resonance imaging (MRI).

The data set can comprise one or more genomic features. The one or more genomic features can comprise a genomic smoking status. The one or more genomic features can comprise gene expression products of genes differentially expressed in subjects that have the cancer and subjects that do not have the cancer. The cancer can be a lung cancer.

The first level of risk of malignancy can be obtained by a first assessment. The first assessment can be a report. The first assessment can be based on a physical examination of the subject. The physical examination can comprise computed tomography scan, non-surgical biopsy, diagnostic bronchoscopy, or a combination thereof. The first level of risk of malignancy can be inconclusive for the cancer.

The subject can have lung nodules that are inconclusive for lung cancer as determined by computed tomography scan or bronchoscopy. The subject can be a current smoker. The subject can be a former smoker. The subject can have a prior history of cancer or can be suspected of having cancer. The subject can not have a prior history of cancer. The subject can have lung nodules that are not results of metastatic lesion in the lung.

The data set can comprise one or more clinical features. The one or more clinical features are selected from the group consisting of: age, gender, smoking status, number of years since subject quit smoking, length of a nodule, infiltrate nodule of the subject, and any combination thereof. The one or more clinical features comprise one or more features selected from the group consisting of: age, gender, smoking status, number of years since subject quit smoking, and length of a nodule.

The data set can comprise one or more gene expression products. The gene expression products can correspond to one or more genes set forth in Table 37, or a derivative thereof.

The method can comprise applying a trained algorithm to the data set to determine the second level of risk of malignancy for having or developing the cancer, and wherein the trained algorithm can be trained with a training data set. The training data set can comprise sequence information derived from transcripts of bronchial epithelial cells. The training data set can comprise sequence information derived from transcripts of nasal epithelial cells. The training data set can comprise gene expression products of one or more genes set forth in Table 37. The training data set can comprise data from samples negative for the cancer and samples positive for the cancer. The training data set can comprise data from samples of current smokers and former smokers. The training data set can comprise data from samples obtained from subjects that have a risk of developing the cancer. The training data set can comprise data from samples obtained from subjects that have a high risk of malignancy based on diagnostic bronchoscopy. The training data set can comprise data from samples obtained from subjects that have a low risk of malignancy based on diagnostic bronchoscopy. The training data set can comprise data from samples obtained from subjects that have an intermediate risk of having the cancer and have only received non-diagnostic bronchoscopy. The training data set can comprise data from samples obtained from subjects that have lung nodules that are inconclusive for lung cancer as determined by computed tomography scan or bronchoscopy.

The subject can have lung nodules that are inconclusive for lung cancer as determined by computed tomography scan or bronchoscopy. The sample can comprise epithelial cells. The sample can comprise epithelial cells from an airway of a subject. The sample can comprise epithelial cells from a mouth, cheek, nose, trachea, or bronchi of a subject. The sample can comprise epithelial cells from a part of an airway of a subject not identified as having a nodule or lesion. The sample can comprise epithelial cells from a histologically normal part of an airway of the subject. The sample can primarily comprise epithelial cells. The sample can comprise nasal epithelial cells or bronchial epithelial cells. The method can further comprise obtaining the sample from the subject by collecting nasal epithelial cells from a nasal passage of the subject or collecting bronchial epithelial cells by bronchial brushing. The nasal epithelial cells can be obtained by nasal swab. The bronchial epithelial cells can be obtained by swab. The first level of risk of malignancy can be based upon identification of nodule(s) or lesion(s) by computed tomography (CT). The nodule(s) or lesion(s) are recommended for diagnostic bronchoscopy. The second level of risk of malignancy can be less than 10%, 9%, 8%, 7%, 6%, 5%, 4%, 3%, 2%, 1%, 0.5%, 0.1%, or lower. The classifier can assign the second level of risk of malignancy with a negative predictive value (NPV) of 90%, 95%, or 99% or higher. The second level of risk of malignancy can be greater than 60%, 65%, 70%, 75%, 80%, 85%, 90%, 95%, or 99%. The classifier can assign the second level of risk of malignancy with a positive predictive value (PPV) of 65%, 70%, 80%, 90%, 99%, or greater.

Disclosed herein is a method, comprising: providing a biological sample of a subject; assaying for expression products of a plurality of genes by hybridizing probes having sequences complementary to the expression products of the plurality of genes to obtain a data set; and in a programed computer, using a classifier to assign the data set corresponding to the sample as negative for lung cancer, wherein the assignment is determined with a negative predictive value greater than 90%.

Disclosed herein is a method, comprising measuring a level of expression of one or more genes from Table 37; and using the level of expression measured in (a) to determine that the subject does not have lung cancer, with a negative predictive value greater than 90%.

Disclosed herein is a system comprising one or more computer processors that are individually or collectively programmed to implement a method, the method comprising: upon obtaining a first level of risk of malignancy of a subject for having or developing a cancer, obtaining a data set corresponding to a sample of the subject; in a programmed computer, using a classifier to assign the data set corresponding to the sample a second level of risk of malignancy for having or developing the cancer; and electronically outputting a report comprising the second level of risk of malignancy of the sample of the subject, wherein the second level of risk of malignancy is determined with a negative predictive value greater than 90%.

The first level of risk of malignancy and the second level of risk of malignancy are different. The second level of risk of malignancy can be greater than the first level of risk of malignancy. The second level of risk of malignancy can be less than the first level of risk of malignancy. The first level of risk of malignancy can be less than 10% and the second level of risk of malignancy can be less than 1%. The first level of risk of malignancy 10% to 60% and the second level of risk of malignancy can be greater than 60%. The first level of risk of malignancy can be greater than 60% and the second level of risk of malignancy greater than 90%.

The subject can have or can be suspected of having a nodule. The nodule can be identified by imaging analysis. The nodule can be identified as having the first level of risk of malignancy of greater than 60% for lung cancer. The nodule can be identified as having the first level of risk of malignancy of less than 10% for lung cancer. The imaging analysis can be low-dose computed tomography (LDCT), computer aided tomography (CAT), or magnetic resonance imaging (MRI).

The data set can comprise one or more genomic features. The one or more genomic features comprise a genomic smoking status. The one or more genomic features comprise gene expression products of genes differentially expressed in subjects that have the cancer and subjects that do not have the cancer. The cancer can be a lung cancer.

The first level of risk of malignancy can be obtained by a first assessment. The first assessment can be a report. The first assessment can be based on a physical examination of the subject. The physical examination can comprise computed tomography scan, non-surgical biopsy, diagnostic bronchoscopy, or a combination thereof. The first level of risk of malignancy can be inconclusive for the cancer.

The subject can have lung nodules that are inconclusive for lung cancer as determined by computed tomography scan or bronchoscopy. The subject can be a current smoker. The subject can be a former smoker. The subject can have a prior history of cancer or can be suspected of having cancer. The subject can not have a prior history of cancer. The subject can have lung nodules that are not results of metastatic lesion in the lung.

The data set can comprise one or more clinical features. The one or more clinical features are selected from the group consisting of: age, gender, smoking status, number of years since subject quit smoking, length of a nodule, infiltrate nodule of the subject, and any combination thereof. The one or more clinical features comprise one or more features selected from the group consisting of: age, gender, smoking status, number of years since subject quit smoking, and length of a nodule.

The data set can comprise one or more gene expression products. The gene expression products correspond to one or more genes set forth in Table 37, or a derivative thereof.

The method can comprise applying a trained algorithm to the data set to determine the second level of risk of malignancy for having or developing the cancer, and wherein the trained algorithm can be trained with a training data set. The training data set can comprise sequence information derived from transcripts of bronchial epithelial cells. The training data set can comprise sequence information derived from transcripts of nasal epithelial cells. The training data set can comprise gene expression products of one or more genes set forth in Table 37. The training data set can comprise data from samples negative for the cancer and samples positive for the cancer. The training data set can comprise data from samples of current smokers and former smokers. The training data set can comprise data from samples obtained from subjects that have a risk of developing the cancer. The training data set can comprise data from samples obtained from subjects that have a high risk of malignancy based on diagnostic bronchoscopy. The training data set can comprise data from samples obtained from subjects that have a low risk of malignancy based on diagnostic bronchoscopy. The training data set can comprise data from samples obtained from subjects that have an intermediate risk of having the cancer and have only received non-diagnostic bronchoscopy. The training data set can comprise data from samples obtained from subjects that have lung nodules that are inconclusive for lung cancer as determined by computed tomography scan or bronchoscopy.

The subject has lung nodules that are inconclusive for lung cancer as determined by computed tomography scan or bronchoscopy. The sample can comprise nasal epithelial cells or bronchial epithelial cells. The first level of risk of malignancy can be based upon identification of nodule(s) or lesion(s) from a CT scan. The identified nodule(s) or lesion(s) can be recommended for diagnostic bronchoscopy. The second level of risk of malignancy can be less than 10% and wherein the classifier assigns the second level of risk of malignancy with a negative predictive value (NPV) of 95% or higher. The second level of risk of malignancy can be greater than 60% and wherein the classifier assigns the second level of risk of malignancy with a positive predictive value (PPV) of 65% or greater.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a diagram outlining a method by which a genomic classifier, as described herein, can be applied to a nasal or bronchial sample from a subject to determine a risk of malignancy of a nodule or lesion after subject is diagnosed with nodules or lesions.

FIG. 2 is a graph depicting the relationship between sensitivity and specificity of a representative model using bronchial samples.

FIG. 3 is a graph depicting the relative AUC of different models using nasal epithelium samples.

FIG. 4 is a graph depicting the specificity obtained from different models using nasal samples.

FIG. 5 is a graph of the specificity of the five classifiers as a measure of validation performance of the five classifiers tested at a sensitivity greater than or equal to 0.95.

FIG. 6 is a graph of the clinical smoking status score generated using the clinical classifier.

FIG. 7 illustrates a comparison of the RIN distribution in nasal brushing samples versus bronchial samples.

FIG. 8 provides a graph of the expression level variation in the 545 nasal brushing samples measured versus the RIN value for reference genes ACTB, GAPDH, AKAP17A and SF3B5.

FIG. 9 provides a graph of the output scores of the clinical factors between nasal brushing samples obtained from subjects diagnosed with either benign or malignant tumors.

FIG. 10 provides a graph of the output scores of the clinical factors between nasal brushing samples obtained from subjects diagnosed with either benign or malignant tumors and further between current and former smokers.

FIG. 11 shows a graph illustrating the score differences obtained using the clinical-genomic classifier between nasal samples obtained from subjects diagnosed with either benign or malignant tumors.

FIG. 12 shows graph of AUC values obtained from different classifiers for all samples and samples obtained from either former or current smokers.

FIG. 13 shows a graph of AUC values obtained from different classifiers for all samples and samples obtained from subjects with nodules less than 3 cm or from subjects with a low/intermediate-test ROM.

FIG. 14 shows a graph of the output scores of the clinical factors between nasal brushing samples obtained from subjects diagnosed with either benign or malignant tumors and further between current and former smokers.

FIG. 15 shows a graph of AUC values obtained from different classifiers for all samples and samples obtained from either former or current smokers.

FIG. 16 shows a graph of AUC values obtained from different classifiers for all samples and samples obtained from subjects with nodules less than 3 cm or from subjects with a low/intermediate-test ROM.

FIG. 17 shows a graph comparing the validation performance, sensitivity versus specificity between the clinical classifier and the clinical-genomic classifier.

FIG. 18 shows a graph of specificity values obtained from different classifiers for all samples and samples obtained from either former or current smokers.

FIG. 19 shows a graph of specificity values obtained from different classifiers for all samples and samples obtained from subjects with nodules less than 3 cm or from subjects with a low/intermediate-test ROM.

FIG. 20 shows a graph of AUC values obtained from different classifiers for all samples and samples obtained from either former or current smokers.

FIG. 21 shows a graph of AUC values obtained from different classifiers for all samples and samples obtained from either former or current smokers.

FIG. 22 shows a graph of specificity values obtained from different classifiers for all samples and samples obtained from either former or current smokers at a sensitivity greater than or equal to 0.95.

FIG. 23 shows a computer system that is programmed or otherwise configured to implement methods provided herein.

FIG. 24 shows a graph of the variation in expression data from cohort samples between current versus former smokers.

FIG. 25 shows a graph of the variation in expression data from cohort samples between samples from subjects diagnosed with malignant or benign tumors.

FIG. 26 shows a graph of the variation of genomic expression between samples obtained at different times.

FIG. 27 shows a graph of the variation of genomic expression between samples obtained from subjects with or without exposure to inhaled medications prior to sample collection.

FIG. 28 illustrates a diagram of the cross-validation procedure used to train the classifier using multiple variables.

FIG. 29 illustrates a diagram of the models used to analyze the clinical features and the genomic features of cohort samples used to train the classifier.

FIG. 30 shows a graph of the variation between the same five patient samples over 37 development plates and 6 verification plates.

FIG. 31 shows a graph of the variation of fifteen different subject samples in relationship to the amount of RNA in each sample.

FIG. 32 illustrates a diagram of the range of risk classification outputs of the classifier.

FIG. 33A illustrates a diagram of the derivation of the study population from the AEGIS I and II cohorts for a validation study

FIG. 33B illustrates a diagram of the derivation of the study population from the Registry cohort for a validation study.

FIG. 34A illustrates the negative predictive value (NPV) of the GSC across different pre-test cancer prevalence in patients who are classified from low to very low risk with specificity of 57.4% and sensitivity of 100%. The prevalence of lung cancer with and without these 45 clinically benign patients was 5.0% and 5.6% in the low pre-test ROM group, respectively

FIG. 34B illustrates the negative predictive value (NPV) of the GSC across different pre-test cancer prevalence in patients who are classified from intermediate to low risk with specificity of 37.3% and sensitivity of 90.6%. The prevalence of lung cancer with and without these 45 clinically benign patients was 28.2% and 34.2% in the intermediate pre-test ROM group, respectively.

FIG. 34C illustrates the positive predictive value (PPV) of the GSC across different pre-test cancer prevalence in patients who are classified from intermediate to high risk with specificity of 94.1% and sensitivity of 28.3%. The prevalence of lung cancer with and without these 45 clinically benign patients was 28.2% and 34.2% in the intermediate pre-test ROM group, respectively.

FIG. 34D illustrates the positive predictive value (PPV) of the GSC across different pre-test cancer prevalence in patients who are classified from high to very high risk with specificity of 91.2% and sensitivity of 34.0%. The prevalence of lung cancer with and without these 45 clinically benign patients was 73.6% and 75.7% in the high pre-test ROM group, respectively.

FIG. 35A illustrates a comparison of the receiver operator curve (ROC) of the GSC in all study patients in the AEGIS I and II cohorts and the Registry.

FIG. 35B illustrates a comparison of the receiver operator curve (ROC) of the GSC in the low and intermediate risk of malignancy study patients in the AEGIS I and II cohorts and the Registry. The asterisk on each curve corresponds to the sensitivity/specificity pair at the decision boundary where patients with scores above the decision boundary will maintain their risk of malignancy; and patients with scores below the decision boundary will have their risk of malignancy down-classified (i.e. low to very low and intermediate to low).

FIG. 35C illustrates a comparison of the receiver operator curve (ROC) of the GSC in the intermediate risk of malignancy study patients in the AEGIS I and II cohorts and the Registry. The asterisk on each curve corresponds to the sensitivity/specificity pair at the decision boundary where patients with scores above the decision boundary will have their risk malignancy up-classified from intermediate to high; and patients with scores below the decision boundary will have their risk of malignancy stay as intermediate.

FIG. 35D illustrates a comparison of the receiver operator curve (ROC) of the GSC in the high risk of malignancy study patients in the AEGIS I and II cohorts and the Registry. The asterisk on each curve corresponds to the sensitivity/specificity pair at the decision boundary where patients with scores above the decision boundary will have their risk malignancy up-classified from high to very high; and patients with scores below the decision boundary will have their risk of malignancy stay as high.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The diagnosis of screen and incidentally detected lung nodules can be challenging. Current guidelines recommend these nodules be managed based upon their probability of malignancy. Patients with nodules having intermediate-risk of malignancy present the biggest diagnostic challenge. Management may include continued imaging surveillance, invasive diagnostic procedures, or surgical resection. Bronchoscopy has a low diagnostic yield for smaller or peripherally located nodules, thus complementary noninvasive diagnostic testing that further stratifies patients may assist in subsequent management decisions.

The Genomic Sequencing Classifier (GSC) is an enhanced second generation classifier that was prospectively developed using a more robust testing platform with richer genomic features from whole transcriptome RNA sequencing in combination with clinical factors. In addition, the GSC was developed with two result thresholds allowing it to serve as both a “rule-in” test and a “rule-out” test, thereby increasing its potential utility in improving risk stratification.

Disclosed herein are non-invasive or minimally invasive assays and related methods that are useful for determining the pathological status of a sample obtained from a subject, which can be used for, as non-limiting examples, diagnosing lung disorder, such as lung cancer, or determining a subject's previous smoking status. Described herein are classifiers, assays and methods that can comprise determining the expression of one or more genes in sample obtained from a subject, for example, a nasal epithelial sample or a bronchial sample. In certain aspects the methods disclosed herein can comprise comparing the expression of one or more of the genes set forth in Table 1 in a sample obtained from a subject to expression of the same genes in a sample of the same tissue type obtained from a control subject. In certain aspects, the assays described herein involves obtaining a sample from a subject's nasal epithelial cells. For example, cells may be taken from the airway of a current or a former smoker (the “field of injury”). This airway may include a nasal passage. In certain aspects, disclosed herein are methods of up- or down-classifying a risk of malignancy for lung cancer in a subject based on analyzing clinical or genomic features of the subject or a sample obtained from the subject. The sample may be obtained from a nasal passage and classification of such a sample may be used to up- or a subject's risk of malignancy for lung cancer, allowing for assessment of risk for lung cancer without requiring invasive sampling procedures. In certain aspects, any of the methods disclosed herein further comprise applying a gene filter to the expression to exclude specimens potentially contaminated with inflammatory cells.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. A human may be an infant, a toddler, a child, a young adult, an adult or a geriatric. A human can be more than about 1, 2, 5, 10, 20, 30, 40, 50, 60, 65, 70, 75, or about 80 years of age.

The subject may have or be suspected of having a disease, such as cancer. The subject may be a smoker, a former smoker or a non-smoker. The subject may have a personal or family history of cancer. The subject may have a cancer-free personal or family history. The subject may be a patient, such as a patient being treated for a disease, such as a cancer patient. The subject may be predisposed to a risk of developing a disease such as cancer. The subject may be in remission from a disease, such as a cancer patient. The subject may be healthy. The subject may exhibit one or more symptoms of lung cancer or other lung disorder (e.g., emphysema, COPD). For example, the subject may have a new or persistent cough, worsening of an existing chronic cough, blood in the sputum, persistent bronchitis or repeated respiratory infections, chest pain, unexplained weight loss and/or fatigue, or breathing difficulties such as shortness of breath or wheezing. The subject may have a lesion, which may be observable by computer-aided tomography (“CT”) or chest X-ray. The subject may be an individual who has undergone a bronchoscopy or who has been identified as a candidate for bronchoscopy (e.g., because of the presence of a detectable lesion, or suspicious or inconclusive imaging result). The subject may be an individual who has undergone an indeterminate or non-diagnostic bronchoscopy. The subject may be an individual who has undergone an indeterminate or non-diagnostic bronchoscopy and who has been recommended to proceed with an invasive lung procedure (e.g., transthoracic needle aspiration, mediastinoscopy, lobectomy, or thoracotomy) based upon the indeterminate or nondiagnostic bronchoscopy. The terms, “patient” and “subject” are used interchangeably herein. The subject may be at risk for developing lung cancer. The subject may be at risk for suffering from a recurrence of lung cancer. The subject may have lung cancer and the assays and methods disclosed herein may be used to monitor the progression of the subject's disease or to monitor the efficacy of one or more treatment regimens.

The term “disease,” as used herein, generally refers to any abnormal or pathologic condition that affects a subject. Examples of a disease include cancer, such as, for example, lung cancer. The disease may be treatable or non-treatable. The disease may be terminal or non-terminal. The disease can be a result of inherited genes, environmental exposures, or any combination thereof. The disease can be cancer, a genetic disease, a proliferative disorder, or others as described herein.

The term “disease diagnostic,” as used herein, generally refers to diagnosing or screening for a disease, to stratify a risk of occurrence of a disease, to monitor progression or remission of a disease, to formulate a treatment regime for the disease, or any combination thereof. A disease diagnostic can include a) obtaining information from one or more tissue samples from a subject, b) making a determination about whether the subject has a particular disease based on the information or tissue sample obtained, c) stratifying the risk of occurrence of the disease, or risk of malignancy, in the subject, including up- or down-classifying a risk of occurrence or malignancy for a subject (e.g., intermediate risk down-classified to low-risk, or intermediate risk up-classified to high risk), and, optionally, d) confirming whether the tissue sample from the subject is positive or negative for a lung disorder (e.g., lung cancer). The disease diagnostic may inform a particular treatment or therapeutic intervention for the disease. The disease diagnostic may also provide a score indicating for example, the severity or grade of a disease such as cancer, or the likelihood of an accurate diagnosis, such as via a p-value, a corrected p-value, or a statistical confidence indicator. The methods disclosed herein may also indicate a particular type of a disease.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### INTRODUCTION

The assays and methods disclosed herein provide classifiers of genomic features, e.g. an expression profile of genes described herein, and clinical features described herein that may be used to assess the risk of malignancy for diseases or disorders, including lung cancer (e.g., adenocarcinoma, squamous cell carcinoma, small cell cancer or non-small cell cancer) when clinical assessment alone is inconclusive for individuals with intermediate risk. Additionally, the assays and methods disclosed herein may provide for classification of whether a subject is a current or former smoker based in part on gene expression products obtained from cells sampled from a nasal or bronchial epithelium. The assays and methods disclosed herein, whether used alone or in combination with other methods, may provide useful information for health care providers to assist them in making early diagnostic and therapeutic decisions for a subject, thereby improving the likelihood that the subject's disease may be effectively treated. Methods and assays disclosed herein may be employed in instances where other methods have failed to provide useful information regarding the lung cancer status of a subject, or to obviate a need for more invasive procedures.

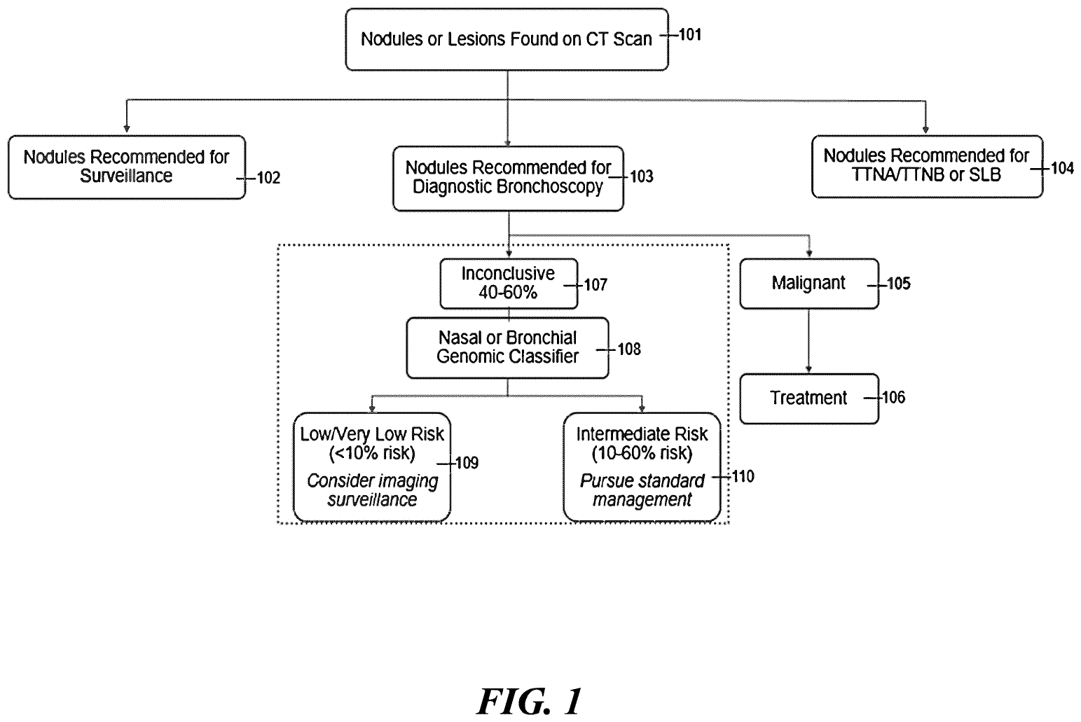

Techniques for obtaining genomic information for lung nodule differential diagnosis may involve using messenger RNA (“mRNA”) transcript expression levels to categorize nodules or lesions detected in the lungs of a subject 101 (e.g., via CT scan) and which are recommended for diagnostic bronchoscopy 103 and are inconclusive 107 as more benign or suspicious, for example, either low or very low risk 109 (down-classifying) or intermediate risk 110 (up-classifying), as demonstrated in FIG. 1.

Altered messenger RNA expression can occur for several reasons, including complex upstream interactions that occur because of sequence changes in key core genes or in relevant peripheral genes, the effect of epigenetic changes that occur without DNA sequence alterations, and both internal and external modifiers, such as inflammation and lifestyle or environment.

The assays and methods disclosed herein may be characterized by the accuracy with which they can discriminate a pathological state, for example, lung cancer from non-lung cancer and their non-invasive or minimally-invasive nature. The assays and methods disclosed herein may be based on detecting differential expression of one or more genes in nasal epithelial cells and such assays and methods may be based on the discovery that such differential expression in nasal epithelial cells are useful for diagnosing cancer in the distant lung tissue. For example, lesions or nodules that are suspicious for lung cancer, or those identified by chest imaging, may be inconclusive and require the decision to follow up with surveillance imaging or a more invasive evaluation. Non-diagnostic bronchoscopy often requires subsequent invasive testing approaches, such as surgical bronchoscopy or biopsy, especially in subjects with intermediate pre-test likelihood of having cancer, even though the lesion may turn out benign. Bronchoscopy may also lack sensitivity in detecting likelihood of cancer in patients with intermediate risk of having cancer when lesion or nodules are small, peripheral, or early stage. As illustrated in FIG. 1, nodules or lesions may be found on the lungs of a subject undergoing a CT scan 101. Based on the results of a CT scan, the CT-identified nodules or lesions may be recommended for surveillance 102, recommended for diagnostic bronchoscopy 103, or recommended for an invasive biopsy, such as transthoracic needle aspiration (TTNA) biopsy or surgical lung biopsy 104. For nodules recommended for diagnostic bronchoscopy, some may be determined to be malignant 105 from the bronchoscopy itself and the subject may be provided treatment 106. However, for a large portion of subjects that undergo bronchoscopy 103, many may receive inconclusive results (e.g., a non-diagnostic bronchoscopy). For such subjects, a nasal or bronchial classifier may be used to analyze gene expression products obtained by analyzing nucleic acid sequences of nasal or bronchial epithelial cells, respectively, and re-classify the subject's risk of having lung cancer. By reclassifying a subject, the individual may avoid more invasive, and costly, medical procedures (e.g., surgical biopsy) which may otherwise be used to obtain more conclusive results. The methods described herein may use genomic and/or clinical classifiers to re-classify the risk of malignancy in a subject. This may obviate a need for more invasive testing approaches mentioned above.

Described herein are methods that may classify a subject's risk of malignancy based on one or more clinical features and/or one or more genomic features, including a gene expression profile of one or more in bronchial epithelial cells or nasal epithelial cells obtained from the subject. The expression profile (e.g., levels and/or transcript sequences) may be used to assess a sample of a subject with inconclusive risk of malignancy 107 and down-classify the risk of malignancy as low or very low (e.g., less than 10%) based on a high negative predictive value (NPV) 109, as illustrated in FIG. 1. Accordingly, a subject re-classified as having low or very low risk of malignancy may be able to avoid undergoing invasive diagnostic procedures. Additionally, a classifier using gene expression profiles of bronchial, nasal, or other cells or tissues may re-classify a subject's sample with inconclusive risk of malignancy as having intermediate 110 (FIG. 1) with risk of malignancy based on a high positive predictive value (PPV). A subject having a first level of risk of malignancy that is intermediate or a CT scan showing inconclusive results 103 may be classified 108 as low risk of malignancy (less than 10% risk, 109), and then may undergo active surveillance with the use of imaging, as illustrated in FIG. 1. A subject having a first level of risk of malignancy that is intermediate or a CT scan showing inconclusive results 103 may be classified 108 as having a intermediate risk of malignancy (10%-60% risk of malignancy, 110), and then may pursue standard management, as illustrated in FIG. 1.

A subject assigned with high or very high risk of malignancy may then undergo further testing, such as surgical bronchoscopy or biopsy, or receive subsequent treatment (e.g. chemotherapy, radiation therapy, immunotherapy, surgical intervention, or combinations thereof) as needed 104, 105, 109, illustrated in FIG. 1.

Accordingly, methods and classifiers provided herein may be used for a substantially less invasive method for diagnosis, prognosis and follow-up of cancer using genomic and/or clinical classifiers. In addition, methods and classifiers provided herein may be used for identification of subjects as appropriate candidates for active surveillance imaging based on low risk of malignancy assigned by the genomic or clinical classifiers.

### Methods for Generating Classification for Samples

The present disclosure provides methods for processing or analyzing a sample of a subject to generate a classification of the sample as benign, suspicious for malignancy, or malignant. In an aspect, methods provided herein may be used for analyzing a sample of a subject to generate a fine-tuned classification of the risk of malignancy. For example, a sample of intermediate risk prior to the classification may be up-classified as of high risk or down-classified as of low risk or very low risk. Such methods may comprise obtaining a plurality of gene expression products from an inconclusive sample and using an algorithm to analyze the gene expression products to classify the sample as benign, suspicious for malignancy, or malignant. In some cases, a plurality of gene expression products may comprise sequences corresponding to mRNA transcripts, mitochondrial transcripts, chromosomal loss of heterozygosity, DNA variants and/or fusion transcripts.

The subject may have undergone an indeterminate or non-diagnostic bronchoscopy. For example, the subject may have undergone an indeterminate or non-conclusive bronchoscopy where the risk of having lung cancer is intermediate. In an aspect, the method may comprise determining that the subject does not have lung cancer, or has a lower risk of having lung cancer, based on the expression levels of one or more (such as, e.g., 2 or more) of the genes set forth in Table 1 in a subject's nasal epithelial cells or bronchial epithelial cells. The methods provided herein may be used to determine that the subject has low or very low risk of having lung cancer (e.g., less than 10% ROM) based on the expression levels of one or more genes set forth in Table 1. Alternatively, the method provided herein may be used to determine that the subject has high or very high risk of having lung cancer based on expression levels of one or more genes set forth in Table 1. In another aspect, the method provided herein may be used to determine that the subject has or does not have lung cancer based on the expression levels in a nasal epithelial cell sample from the subject of one or more (such as, e.g., 2, 3, 4, 5, 6, 7, 8, 9, 10, 11, 12, 13, 14, 15, 16, 17, 18, 19, 20, 21, 22, 23, 24, or 25) genes listed in Table 3, or the subject has low or very low risk of having lung cancer based on the expression levels of one or more (such as, e.g., 2, 3, 4, 5, 6, 7, 8, 9, 10, 11, 12, 13, 14, 15, 16, 17, 18, 19, 20, 21, 22, 23, 24, or 25) genes set forth in Table 3. In some embodiments, the method provided herein may be used to determine that the subject has high or very high risk of having lung cancer (e.g., greater than 60% ROM) based on expression levels of one or more genes set forth in Table 3.

Also contemplated are methods for determining a genomic smoking status of an individual which may be used as an input to a nasal or bronchial classifier, as described here. In some examples, the method may comprise determining a pathological status, e.g., smoking status, of a subject base on the expression levels of one or more genes set forth in Table 2. For example, the method may determine whether a subject is a current or a former smoker based on the expression levels of one or more genes set forth in Table 2 in a sample of the subject.

In some examples, the method may use a trained algorithm that comprises one or more classifiers and is implemented by one or more programmed computer processors to process the expression gene products to generate a classification of sample of a pathological state. The sample may be classified by risk profile. For example, the sample may be stratified as being of very high, high, low, very low, or intermediate risk of being malignant in a second level of risk of malignancy. This risk stratification may be an up- or down-classification relative to what was previously classified as an inconclusive or intermediate risk sample in the first level of risk of malignancy. This re-classification, in turn, may be used to inform monitoring or treatment discussion for the subject from which the sample was obtained.

The algorithm may be a trained algorithm. The algorithm may be trained using reference samples (e.g., an algorithm that is trained on at least 10, 200, 100 or 500 reference samples). Reference samples may be obtained from subjects having been diagnosed with the disease or from healthy subjects. A risk of malignancy may be assigned to the reference samples. The algorithm may also be trained using clinical features (e.g., age, gender, smoking status, smoking history, number of year since quit smoking, nodule length, nodule size, shape of nodule, lesions, or combinations thereof) or genomic features (e,g., expression profiles or products of genes differentially expressed benign samples, expression profiles or products of genes differentially expressed in malignant samples, expression profiles or products of genes differentially expressed in current smokers, expression profiles or products of genes differentially expressed in former smokers, genomic smoking status or index, expression of one or more genes as set forth in Table 1, Table 2, or Table 3) from the reference samples or subject that the sample is obtained therefrom. The trained algorithm may be trained with a combination of clinical and genomic features. The trained algorithm may process the sequence information of expression gene products corresponding to about 10,000 genes. The trained algorithm may process the sequence information of expression gene products corresponding to at least 2 genes of Table 1. The trained algorithm may process the sequence information of expression gene products corresponding to at least 3 genes of Table 1. The trained algorithm may process the sequence information of expression gene products corresponding to at least 4 genes of Table 1. The trained algorithm may process the sequence information of expression gene products corresponding to at least 5 genes of Table 1. The trained algorithm may process the sequence information of expression gene products corresponding to at least 6 genes of Table 1. The trained algorithm may process the sequence information of expression gene products corresponding to at least 7 genes of Table 1. The trained algorithm may process the sequence information of expression gene products corresponding to at least 8 genes of Table 1. The trained algorithm may process the sequence information of expression gene products corresponding to at least 10 genes of Table 1. The trained algorithm may process the sequence information of expression gene products corresponding to at least 11 genes of Table 1. The trained algorithm may process the sequence information of expression gene products corresponding to at least 12 genes of Table 1. The trained algorithm may process the sequence information of expression gene products corresponding to at least 13 genes of Table 1. The trained algorithm may process the sequence information of expression gene products corresponding to at least 14 genes of Table 1. The trained algorithm may process the sequence information of expression gene products corresponding to at least 15 genes of Table 1. The trained algorithm may process the sequence information of expression gene products corresponding to at least 16 genes of Table 1. The trained algorithm may process the sequence information of expression gene products corresponding to at least 17 genes of Table 1. The trained algorithm may process the sequence information of expression gene products corresponding to at least 18 genes of Table 1. The trained algorithm may process the sequence information of expression gene products corresponding to at least 19 genes of Table 1. The trained algorithm may process the sequence information of expression gene products corresponding to at least 20 genes of Table 1. The trained algorithm may process the sequence information of expression gene products corresponding to at least 21 genes of Table 1. The trained algorithm may process the sequence information of expression gene products corresponding to at least 22 genes of Table 1.

The methods disclosed herein may include extracting and analyzing nucleic acids (e.g. RNA or DNA) from one or more samples from a subject. Nucleic acids can be extracted from the entire sample obtained or can be extracted from a portion of the sample. In some cases, the portion of the sample not subjected to nucleic acid extraction may be analyzed by cytological examination or immunohistochemistry. Methods for RNA or DNA extraction from biological samples can include for example phenol-chloroform extraction (such as guanidinium thiocyanate phenol-chloroform extraction), ethanol precipitation, spin column-based purification, or others. Isolated RNA may further be purified, or whole cells containing RNA may be directly placed into microfluidic devices for gene expression and/or sequencing analysis.

As set forth in the present disclosure, an expression level of one or more genes of gene expression products can be obtained by assaying for an expression level. Assaying may comprise array hybridization, nucleic acid sequencing, nucleic acid amplification, or others. Assaying may comprise sequencing, such as DNA or RNA sequencing. Such sequencing may be by next generation (NextGen) sequencing, such as high throughput sequencing or whole genome sequencing (e.g., Illumina). Such sequencing may include enrichment. Assaying may comprise reverse transcription polymerase chain reaction (PCR). Assaying may utilize markers, such as primers, that are selected for each of the one or more genes of the first or second sets of genes. Additional methods for determining gene expression levels may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immuno-absorbance assays, mass-spectrometry, immunohistochemistry, blotting, sequencing, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Gene expression product levels may be normalized to an internal standard such as total messenger ribonucleic acid (mRNA) or the expression level of a particular gene.

RNA (e.g., mNA) may be analyzed by expression profiling, for example, by array-based gene expression profiling. Non-limiting examples of techniques for determining gene expression levels include RT-PCR, DNA microarray hybridization, RNASeq, or a combination thereof. One or more of the gene expression products may be labeled. For example, a mRNA (or a cDNA made from such an mRNA) from a nasal epithelial cell sample may be labeled. In an example, RNA expression can be analyzed with Northern-blot hybridization, ribonuclease protection assay, or reverse transcriptase polymerase chain reaction (RT-PCR) based methods. A number of quantitative RT-PCR based methods have been described and are useful in measuring the amount of transcripts according to the present disclosure. These methods include RNA quantification using PCR and complementary DNA (cDNA) arrays (Shalon, et al, Genome Research 6(7):639-45, 1996; Bernard, et al, Nucleic Acids Research 24(8): 1435-42, 1996), real competitive PCR using a MALDI-TOF Mass spectrometry based approach (Ding, et al., PNAS, 100: 3059-64, 2003), solid-phase mini-sequencing technique, which is based upon a primer extension reaction (U.S. Pat. No. 6,013,431, Suomalainen, et al., Mol. Biotechnol. June; 15(2): 123-31, 2000), ion-pair high-performance liquid chromatography (Doris, et al., J. Chromatogr. A May 8; 806(1):47-60, 1998), and 5′ nuclease assay or real-time RT-PCR (Holland, et al, Proc Natl Acad Sci USA 88: 7276-7280, 1991).

### Risk of Malignancy

In an aspect, the methods disclosed herein may involve classifying the gene expression information and/or clinical information obtained from a subject. A subject may have nodules or lesions based on a computed tomography scan. The subject may have undergone a non-diagnostic bronchoscopy. The subject may have undergone a diagnostic bronchoscopy. A subject may have been assessed with a risk of malignancy, for example, risk of having lung cancer based on clinical information such as age, smoking history, and/or size, position, and shape of nodules. Physicians can make assessment of an individual's risk of having or developing cancer based on clinical test results and examinations. For example, a physician can assess the risk of malignancy based on any lesion or nodule detected with a CT scan or chest radiography. The lesion or nodule may be characterized, for example, based on whether the nodule is solid, part solid, or nonsolid (e.g. pure ground glass nodules), whether the nodule is calcified, the size of the nodule (e.g., less than 1, 2, 3, 4, 5, 6, 7, 8 mm in diameter or more than 8 mm in diameter), and may combine evidence with different diagnosis approaches including PET scan, CT scan, chest radiography, or non-surgical biopsy. A physician's assessment of risk of malignancy may be included in a report. In one non-limiting example, the pre-classifier test risk of malignancy based on clinical factors may be determined by the following equations:

Probability of malignancy=e^{x}/(1+e^{x}), wherein x=−6.8272+(0.0391×age)+(0.7917×smoke)+(1.3388×cancer)+(0.1274×diameter)+(1.0407×spiculation)+(0.7838×location)

where e is the base of natural logarithms, age is the subject's age in years, smoke=1 if the subject is a current or former smoker (otherwise=0), cancer=1 if the subject has a history of an extrathoracic cancer that was diagnosed >5 years ago (otherwise=0), diameter is the diameter of the nodule in millimeters, spiculation=1 if the edge of the nodule has spicules (otherwise=0), and location=1 if the nodule is located in an upper lobe (otherwise=0).

Clinical evaluation of risks is further described in Gould et al., Chest (2013) 143(5 Suppl): e93S-e120S, and this reference is incorporated herein by reference in its entirety.

Accordingly, the methods provided herein may involve re-classifying a risk of malignancy level based on a sample of a subject. This may include obtaining a first level of risk of malignancy for a subject. The first level of risk of malignancy may be a pre-test risk of malignancy. The pre-test risk of malignancy may refer to risk assessments performed prior to classification methods described in the present disclosure. It can include, for example, detection of nodules or lesions on a CT scan, performing a bronchoscopy, and/or determining a risk of malignancy as set forth above, in accordance with Gould et al. 2013. Pre-test bronchoscopy results may be inconclusive or non-diagnostic. Using the methods described herein, the first level of risk of malignancy may be reclassified to a second level of risk of malignancy. In re-classification, the methods described herein may up-classify or down-classify the first level to the second level of risk of malignancy. In one example shown in FIG. 1, for inconclusive or pre-test intermediate risk samples having a first level or pre-test ROM of 10-60%, up- or down-classification may down-classify a subject as low risk (ROM of less than 10%) thereby allowing the subject to forgo potentially invasive follow-up procedures. In another example shown in FIG. 1, up-classification using the methods described herein of a pre-test intermediate or inconclusive sample (e.g., wherein a first level of risk of malignancy is intermediate, based on a ROM calculation described above), the methods described herein may identify that a subject has intermediate risk for which standard management strategies may be required.

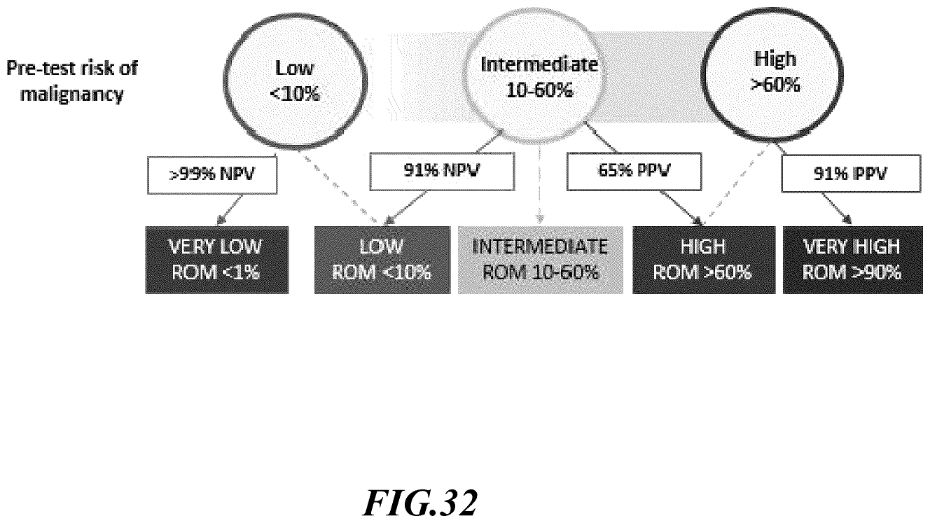

A non-limiting example is illustrated in FIG. 32. For instance, clinical evaluation (e.g., a first level, or pre-test, risk of malignancy) may assign a subject with a low risk of malignancy.

A low pre-test risk of malignancy (e.g., less than 10%) may be re-classified from low (less than 10% to 1%) to very low (less than 1%). Classification from pre-test low to low or very low may be based on in part on expression levels of one or more genes in Table 1 or Table 3 or Table 37. A low pre-test risk of malignancy may be re-classified from low to intermediate. Re-classfication from pre-test low to intermediate may be based in part on expression levels of one or more genes in Table 1 or Table 3 or Table 37.

A sample of an individual may have been assigned with intermediate pre-test risk of malignancy (e.g., between 10% and 60%) by clinical tests before assessment with the genomic or clinical genomic classifiers described herein. In such cases, the intermediate risk of malignancy may be re-classified from intermediate to low risk (e.g., less than 10%). This may be based in part on expression levels of one or more genes in Table 1 or Table 3 or Table 37. A intermediate risk of malignancy may be re-classified from intermediate to high risk (e.g., greater than 60%). This may be based in part on expression levels of one or more genes in Table 1 or Table 3 or Table 37.

Clinical evaluation may assign a subject with a pre-test high risk of malignancy (e.g., more than 60%). An individual with high pre-classifying risk of malignancy may be up-classified as having very high risk of malignancy (e.g., >90%) or down-classified as intermediate risk of malignancy (e.g., between 10%-60%). This may be based in part on expression levels of one or more genes in Table 1 or Table 3 or Table 37.

The trained algorithm may comprise a genomic classifiers, a clinical classifier, or both. The likelihood that the subject has lung cancer, or the risk of malignancy, may also be determined based on the presence or absence of one or more clinical risk factors or diagnostic indicia of lung cancer, such as the results of imaging studies. As used herein, the “likelihood of cancer” is used interchangeably with “risk of malignancy (ROM)” to refer to the probability of a subject having or developing a cancer, for example, a lung cancer.

A risk of malignancy may be determined based in part on clinical features or clinical risk factors. As used herein, the term “clinical risk factors” or “clinical factors” refer broadly to any diagnostic indicia (e.g., subjective or objective diagnostic criteria) that may be relevant for determining a subject's risk of having or developing lung cancer. Examples of clinical risk factors that may be used in combination with the methods or assays disclosed herein may include, but not limited to, for example, imaging studies (e.g., chest X-ray, CT scan, etc.), presence of nodule, lesion, the size, shape, and/or position of lung nodules, the subject's smoking status or smoking history and/or the subject's age. Clinical risk factors may be used as clinical features which are used to classify a sample obtained from a subject. A trained algorithm may also be trained using clinical features that correspond to one or more clinicial risk factors. As such, clinical features may include results from imaging studies (e.g., chest X-ray, CT scan, etc.), presence of nodule, lesion, the size, shape, and/or position of lung nodules, the subject's smoking status or smoking history and/or the subject's age. In certain aspects, when such clinical risk factors are combined with the methods and assays disclosed herein, the predictive power of such methods and assays may be further enhanced.

The risk of malignancy (“ROM”) for lung cancer may be determined based on one or more genomic features. The one or more genomic features may include, for example, a gene expression profile of one or more genes in a sample of the subject. This may include one or more genes disclose herein. For example, the one or more genomic features may comprise certain groups of genes expressed in cells obtained from a nasal sample or a bronchial sample, and which may be analyzed in an expression profile of a subject's sample.

The classifiers described herein may comprise one or more genomic features such as expression profile of genes as described herein and one or more clinical features. The genomic features may comprise expression levels or transcript levels of one or more of the genes set forth in Table 1 or Table 3 or Table 37 in a sample as compared to a reference or a control sample. The genomic features may also comprise a genomic smoking index, for example, a smoking index based on analysis of genes of expression profile of one or more genes as set forth in Table 2.

Differential expression of the one or more genes may be determined with reference to the one or more of the genes set forth in Table 1 or Table 3 or Table 37. As used herein, the term “differential expression” may be used to refer to any qualitative or quantitative differences in expression of the gene or differences in the expressed gene product (e.g., mRNA) in a sample of the subject (e.g. the nasal epithelial cells of the subject). A differentially expressed gene may qualitatively have its expression altered, including an activation or inactivation, in, for example, the presence of absence of cancer and, by comparing such expression in nasal epithelial cell to the expression in a control sample in accordance with the methods and assays disclosed herein, the presence or absence of lung cancer may be determined.

In an aspect, also disclosed herein is a group of genes (e.g., one or more of the genes listed in Table 1, Table 3, or Table 37) that may be analyzed to determine the presence or absence of lung cancer (e.g., adenocarcinoma, squamous cell carcinoma, small cell cancer and/or non-small cell cancer) from a biological sample comprising the subject's nasal epithelial cells. The present disclosure also provides a group of genes (e.g., Table 2) that may be analyzed to determine a subject's smoking status from a biological sample comprising the subject's nasal epithelial cells. For example, expression of one or more genes listed in Table 1 or Table 3 or Table 37 or Table 37 may be assayed to determine whether the subject has or is at risk of developing lung cancer. In another example, expression of one or more genes listed in Table 1 or Table 3 or Table 37 may be assayed to assess a risk of malignancy for lung cancer and expression of one or more genes listed in Table 2 may be assayed to generate a smoking status index which may also factor into the risk of malignancy assessment.

A sample obtained from a subject may comprise cells obtained from different tissues of a subject, for example, nasal epithelial cells or bronchial epithelial cells. Nasal or bronchial epithelial cells may be analyzed using at least one gene listed in Table 1 or Table 37. For example, expression of about 1, 2, 3, 4, 5, 6, 7, 8, 9, 10, 10-15, 15-20, 20-30, 30-40, 40-50, 50-60, 60-70, 70-80, 80-90, 90-100, or at least 10, at least 20, at least 22, of the genes of a sample of a subject as listed in Table 1 or Table 37 may be measured to determine the risk level of lung cancer of the subject. Expression of 1, 2, 3, 4, 5, 6, 7, 8, 9, 10, 11, 12, 13, 14, 15, 16, 17, 18, 19, 20, 21, 22, 23, 24, 25, or 26 genes of a sample of a subject as listed in Table 3 or Table 37 may be measured to determine the risk level of lung cancer of the subject. In another example, about 1, 2, 3, 4, 5, 6, 7, 8, 9, 10, 10-15, 15-20, 20-30, 30-40, 40-50, 50-60, 60-70, 70-80, 80-90, 90-100, or at least 10, at least 20, at least 30, at least 40 at least 50, at least 60, at least 70, at least 80, at least 90, at least 100, at least 110, at least 120, at least 130, at least 140, at least 150, at least 160, at least or at maximum of 170, at least or at maximum of 180, at least or at maximum of 190, at least or at maximum of 200, 210, 220, 230, 240, or 248 of the genes of a sample of a subject as listed in Table 2 may be measured to determine the smoking status of the subject.

Detection of lung cancer in a sample from a subject can be accomplished by processing the expression of the genes or groups of genes set forth in, for example Table 1 or Table 3 or Table 37, in the subject's cells, e.g. nasal epithelial cells, against a control subject or a control group (e.g., a positive control with a confirmed diagnosis of lung cancer). Processing may include applying a trained algorithm to one or more clinical and/or genomic features of a subject. Control samples (e.g., samples determined to be positive or negative for lung cancer) may be used to train an algorithm, which algorithm can then classify a subject's sample.

In certain aspects, the determination of a subject's smoking status, or of a genomic smoking index, can be made by processing expression of the genes or groups of genes from the subject's cells, e.g. nasal epithelial cells, against a control subject or a control group (e.g., a non-smoker negative control, or a smoker positive control).

An appropriate control or reference may be an expression level (or range of expression levels) of a particular gene that is indicative of a known lung cancer status in a comparable control sample, for example, a sample of the same tissue or cell type obtained with same methods. An appropriate reference can be determined experimentally by a practitioner of the methods disclosed herein or may be a pre-existing expression value or range of values.

The control groups can be or can comprise one or more subjects with a positive lung cancer diagnosis, a negative lung cancer diagnosis, non-smokers, smokers and/or former smokers. Preferably, the genes or their expression products of the subject may be compared relative to a similar group, except that the members of the control groups may not have lung cancer. For example, such a comparison may be performed in the nasal epithelial cell sample from a smoker relative to a control group of smokers who do not have lung cancer. Such a comparison may also be performed, e.g., in the nasal epithelial cell sample from a non-smoker relative to a control group of non-smokers who do not have lung cancer. Similarly, such a comparison may be performed in the nasal epithelial cell sample from a former smoker or a suspected smoker relative to a control group of smokers who do not have lung cancer. The transcripts or expression products may then be compared against the control to determine whether increased expression or decreased expression can be observed, which depends upon the particular gene or groups of genes being analyzed, as set forth, for example, in Table 1 or Table 3 or Table 37. In an aspect, at least 50% of the gene or groups of genes subjected to expression analysis may provide the described pattern. Greater reliability may be obtained as the percent approaches 100%. Accordingly, at least about 55%, 60%, 65%, 70%, 75%, 80%, 85%, 90%, 95%, 98%, 99% of the one or more genes subjected to expression analysis may be needed to demonstrate an altered expression pattern that is indicative of the presence or absence of lung cancer, as set forth in, for example, Table 1 or Table 3 or Table 37. Similarly, at least about 55%, 60%, 65%, 70%, 75%, 80%, 85%, 90%, 95%, 98%, 99% of the one or more genes subjected to expression analysis may be needed to demonstrate an altered expression pattern that is indicative of the subject's smoking status, as set forth in, for example, Table 2.

Any combination of the genes and/or transcripts of Table 1, Table 2, Table 3, or Table 37 can be used in connection with the assays and methods disclosed herein. Any combination of at least 5-10, 10-20, 20-22, genes selected from the group consisting of genes or transcripts as shown in the Table 1 or Table 37. A combination of genes used to classify the risk of lung cancer of a subject may be a subset of Table 1 or Table 37. For example, a combination of genes used to classify the risk of lung cancer of a subject may be a selected subset of Table 1 or Table 37 that provides enhanced diagnostic power as compared to a gene combination of the same number of genes randomly taken from Table 1 or Table 37. A combination of genes used to classify the risk of lung cancer of a subject may comprise the genes in Table 3 or Table 37. A combination of genes used to classify the risk of lung cancer may be a subset of Table 3 or Table 37. Similarly, a combination of genes used to classify the smoking status of a subject may be a subset of Table 2.

The analysis of the gene expression of one or more genes may be performed using any of a variety of gene expression methods. Such methods include but are not limited to expression analysis using nucleic acid chips (e.g. Affymetrix chips) and quantitative RT-PGR based methods using, for example real-time detection of the transcripts. Analysis of transcript levels according to the present disclosure can be made using total or messenger RNA or proteins encoded by the genes identified in the diagnostic gene groups of the present disclosure as a starting material. The analysis may be performed analyzing the amount of proteins encoded by one or more of the genes listed in Table 1, Table 2 or Table 3 and present in the sample. The analysis may also comprise an immunohistochemical analysis with an antibody directed against one or more proteins encoded by the genes and/or transcripts as shown in Table 1, Table 2, Table 3 or Table 37.

Analysis may be performed using DNA by analyzing the gene expression regulatory regions of the airway transcriptome genes using nucleic acid polymorphisms, such as single nucleic acid polymorphisms or SNPs, wherein polymorphisms known to be associated with increased or decreased expression are used to indicate increased or decreased gene expression in the individual.

The methods provided herein can be used to determine if nasal epithelial cell gene expression profiles are affected by lung cancer. The methods disclosed herein can also be used to identify patterns of gene expression that are diagnostic of a pathological state, for example, risk of malignancy or smoking status. All or a subset of the genes identified according to the methods described herein can be used to design an array, for example, a microarray, specifically intended for the diagnosis or prediction of lung disorders or susceptibility to lung disorders. The efficacy of such custom-designed arrays can be further tested, for example, in a large clinical trial of smokers.

### Samples

As used herein, a sample or a biological sample can be used to refer to any sample taken or derived from a subject. A sample may comprise one or more cells, for example, nasal epithelial cells. A sample obtained from a subject can comprise tissue, cells, cell fragments, cell organelles, nucleic acids, genes, gene fragments, expression products, gene expression products, gene expression product fragments or any combination thereof. A sample can be heterogeneous or homogenous. A sample can comprise blood, urine, cerebrospinal fluid, seminal fluid, saliva, sputum, stool, lymph fluid, tissue, or any combination thereof. A sample can be a tissue-specific sample such as a sample obtained from a thyroid, skin, heart, lung, kidney, breast, pancreas, liver, muscle, smooth muscle, bladder, gall bladder, colon, intestine, brain, esophagus, or prostate. A sample of the present disclosure can be obtained by various methods, such as, for example, fine needle aspiration (FNA), core needle biopsy, vacuum assisted biopsy, incisional biopsy, excisional biopsy, punch biopsy, shave biopsy, skin biopsy, or any combination thereof. The sample can be obtained from a region of a subject's airway not identified as having a lesion or nodule. The sample can be obtained from a histologically normal party of a subject's airway.

The subject can have a nodule or lesion identified by imaging analysis. The imaging analysis can be computed tomography (CT), low dose CT (LDCT), computer assisted tomography (CAT), X-ray, magnetic resonance imaging (MRI), etc.

If a nodule or lesion is observed in a left lobe of the lung and not the right lobe of the lung, the sample can be obtained from the bronchus or right lobe of the lung. The sample can be substantially epithelial cells from the bronchi of the right lobe of the lung. The sample can be obtained by bronchial brushing.

If a nodule or lesion is observed in a right lobe of the lung and not the left lobe of the lung, the sample can be obtained from the bronchus or left lobe of the lung. The sample can be substantially epithelial cells from the bronchi of the left lobe of the lung. The sample can be obtained by bronchial brushing.

The methods and assays disclosed herein can be characterized as being much less invasive relative to, for example, bronchoscopy. A biological sample may be obtained (e.g., at a point-of-care facility, a physician's office, a hospital) by procuring a tissue or fluid sample from a subject. A biological sample may be obtained from a subject by another individual or entity, such as a healthcare (or medical) professional or robot. A medical professional can include a physician, nurse, medical technician or other. In some cases, a physician may be a specialist, such as an oncologist, surgeon, or endocrinologist. A medical technician may be a specialist, such as a cytologist, phlebotomist, radiologist, pulmonologist or others. In some cases, a medical professional need not be involved in the initial diagnosis of a disease or the initial sample acquisition. An individual, such as the subject, may alternatively obtain a sample through the use of an over the counter kit. The kit may contain collection unit or device for obtaining the sample as described herein, a storage unit for storing the sample ahead of sample analysis, and instructions for use of the kit.

A sample can be obtained a) pre-operatively, b) post-operatively, c) after a cancer diagnosis, d) during routine screening following remission or cure of disease, e) when a subject is suspected of having a disease, f) during a routine office visit or clinical screen, g) following the request of a medical professional, or any combination thereof. Multiple samples at separate times can be obtained from the same subject, such as before treatment for a disease commences and after treatment ends, such as monitoring a subject over a time course. Multiple samples can be obtained from a subject at separate times to monitor the absence or presence of disease progression, regression, or remission in the subject.

A biological sample may be obtained from a subject (e.g., a subject at risk for lung cancer) using a brush or a swab. The sample may comprise nasal epithelial cells. For example, a nasal epithelial cell sample is collected from a subject by nasal brushing or swabbing. The nasal epithelial cell sample may be collected by brushing the inferior turbinate and/or the adjacent lateral nasal wall. For example, following local anesthesia with 2% lidocaine solution, a CYROBRUSH© (MedScand Medical, Malm5, Sweden) or a similar device, is inserted into the nare of the subject, for example the right nare, and under the inferior turbinate using a nasal speculum for visualization. The brush or swab may be turned (e.g., turned 1, 2, 3, 4, 5 times or more) to collect the nasal epithelial cells, which may then be subjected to analysis in accordance with the assays and methods disclosed herein.

The biological sample may or may not comprise cells from a bronchial airway. For example, bronchial airway epithelial cell sample may be obtained by bronchial brushing. Bronchial samples may be collected during bronchoscopy using a standard cytologic brush through the bronchoscope that brushes the bronchial wall. Qiagen's ProtectCell RNA preservative may be used to preserve the samples. The airway epithelial cells, in preservative may then be used for RNA extraction and expression or sequencing analysis. A biological sample also may not include or comprise bronchial airway epithelial cells. For example, in certain instances, the biological sample may not include epithelial cells from the mainstem bronchus. In certain aspects, the biological sample may not include cells or tissue collected from bronchoscopy. The biological sample may or may not need to include cells or tissue isolated from a pulmonary lesion.

A sample may comprise cells harvested from a tissue, e.g., cells harvested from a nasal epithelial cell sample. The cells may be harvested from a sample using standard techniques known in the art or disclosed herein. For example, cells may be harvested by centrifuging a cell sample and re-suspending the pelleted cells. The cells may be re-suspended in a buffered solution such as phosphate-buffered saline (PBS). After centrifuging the cell suspension to obtain a cell pellet, the cells may be lysed to extract nucleic acid, e.g., messenger RNA. All samples obtained from a subject, including those subjected to any sort of further processing, are considered to be obtained from the subject.

RNA yield or RNA amount of a sample can be measured in nanogram to microgram amounts. An example of an apparatus that can be used to measure nucleic acid yield in the laboratory is a NANODROP® spectrophotometer, QUBIT® fluorometer, or QUANTUS™ fluorometer. The accuracy of a NANODROP® measurement may decrease significantly with very low RNA concentration. Quality of data obtained from the methods described herein can be dependent on RNA quantity. Meaningful gene expression or sequence variant data or others can be generated from samples having a low or un-measurable RNA concentration as measured by NANODROP®. In some cases, gene expression or sequence variant data or others can be generated from a sample having an unmeasurable RNA concentration.

The methods as described herein can be performed using samples with low quantity or quality of polynucleotides, such as DNA or RNA. A sample with low quantity or quality of RNA can be for example a degraded or partially degraded tissue sample. The RNA quality of a sample can be measured by a calculated RNA Integrity Number (RIN) value. The RIN value is an algorithm for assigning integrity values to RNA measurements. The algorithm can assign a 1 to 10 RIN value, where an RIN value of 10 can be completely intact RNA. A sample as described herein that comprises RNA can have an RIN value of about 9.0, 8.0, 7.0, 6.0, 5.0, 4.0, 3.0, 2.0, 1.0 or less. In some cases, a sample comprising RNA can have an RIN value equal or less than about 8.0. In some cases, a sample comprising RNA can have an RIN value equal or less than about 6.0. In some cases, a sample comprising RNA can have an RIN value equal or less than about 4.0. In some cases, a sample can have an RIN value of less than about 2.0.

### Markers and Primers for Hybridization, Sequence, and Amplification

Suitable reagents for conducting array hybridization, nucleic acid sequencing, nucleic acid amplification or other amplification reactions include, but are not limited to, DNA polymerases, markers such as forward and reverse primers, deoxynucleotide triphosphates (dNTPs), and one or more buffers. Such reagents can include a primer that is selected for a given sequence of interest, such as the one or more genes of the first set of genes and/or second set of genes. mRNA may be isolated from a sample is converted to complementary DNA (cDNA) in a hybridization reaction or is used in a hybridization reaction together with one or more cDNA probes. Converted cDNAs may be amplified by polymerase chain reaction (PCR) or other amplification method(s) available to those of ordinary skill in the art.

In such amplification reactions, one primer of a primer pair can be a forward primer complementary to a sequence of a target polynucleotide molecule (e.g. the one or more genes of the first or second sets) and one primer of a primer pair can be a reverse primer complementary to a second sequence of the target polynucleotide molecule and a target locus can reside between the first sequence and the second sequence.

Various methods that may be used for selecting primers for PCR amplification may be used. See, e.g., McPherson et al., PCR Basics: From Background to Bench, Springer-Verlag, 2000, incorporated by reference in their entirety. The length of the forward primer and the reverse primer can depend on the sequence of the target polynucleotide (e.g. the one or more genes of the first or second sets) and the target locus. In some cases, a primer can be greater than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, or about 100 nucleotides in length. As an alternative, a primer can be less than about 100, 95, 90, 85, 80, 75, 70, 65, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or about nucleotides in length. In some cases, a primer can be about 15 to about 20, about 15 to about 25, about 15 to about 30, about 15 to about 40, about 15 to about 45, about 15 to about 50, about 15 to about 55, about 15 to about 60, about 20 to about 25, about 20 to about 30, about 20 to about 35, about 20 to about 40, about 20 to about 45, about 20 to about 50, about 20 to about 55, about 20 to about 60, about 20 to about 80, or about 20 to about 100 nucleotides in length.

Primers can be designed according to parameters for avoiding secondary structures and self-hybridization, such as primer dimer pairs. Different primer pairs can anneal and melt at about the same temperatures, for example, within 1° C., 2° C., 3° C., 4° C., 5° C., 6° C., 7° C., 8° C., 9° C. or 10° C. of another primer pair.

The target locus can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends or 5′ ends of the plurality of template polynucleotides.

Markers (i.e., primers) for the methods described can be one or more of the same primer. In some instances, the markers can be one or more different primers such as about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 or more different primers. In such examples, each primer of the one or more primers can comprise a different target or template specific region or sequence, such as the one or more genes of the first or second sets.

One or more primers can comprise a fixed panel of primers. The one or more primers can comprise at least one or more custom primers. The one or more primers can comprise at least one or more control primers. The one or more primers can comprise at least one or more housekeeping gene primers. In some instances, the one or more custom primers anneal to a target specific region or complements thereof. The one or more primers can be designed to amplify or to perform primer extension, reverse transcription, linear extension, non-exponential amplification, exponential amplification, PCR, or any other amplification method of one or more target or template polynucleotides.

Primers can incorporate additional features that allow for the detection or immobilization of the primer but do not alter a basic property of the primer (e.g., acting as a point of initiation of DNA synthesis). For example, primers can comprise a nucleic acid sequence at the 5′ end which does not hybridize to a target nucleic acid, but which facilitates cloning or further amplification, or sequencing of an amplified product. For example, the sequence can comprise a primer binding site, such as a PCR priming sequence, a sample barcode sequence, or a universal primer binding site or others.

A universal primer binding site or sequence can attach a universal primer to a polynucleotide and/or amplicon. Universal primers can include −47F (M13F), alfaMF, AOX3′, AOX5′, BGHr, CMV-30, CMV-50, CVMf, LACrmt, lamgda gt10F, lambda gt 10R, lambda gt11F, lambda gt11R, M13 rev, M13Forward (−20), M13Reverse, male, p10SEQPpQE, pA-120, pet4, pGAP Forward, pGLRVpr3, pGLpr2R, pKLAC14, pQEFS, pQERS, pucU1, pucU2, reversA, seqIREStam, seqIRESzpet, seqori, seqPCR, seqpIRES−, seqpIRES+, seqpSecTag, seqpSecTag+, segretro+PSI, SP6, T3-prom, T7-prom, and T7-termInv. As used herein, attach can refer to both or either covalent interactions and noncovalent interactions. Attachment of the universal primer to the universal primer binding site may be used for amplification, detection, and/or sequencing of the polynucleotide and/or amplicon.

mRNA isolated from a sample may be hybridized to a synthetic DNA probe, which mayincludes a detection moiety (e.g., detectable label, capture sequence, barcode reporting sequence). A non-natural mRNA-cDNA complex may be ultimately made and used for detection of the gene expression product. In another example, mRNA from the sample may be directly labeled with a detectable label, e.g., a fluorophore. In a further example, the non-natural labeled-mRNA molecule may be hybridized to a cDNA probe and the complex is detected.

cDNA may be amplified with primers that introduce an additional DNA sequence (e.g., adapter, reporter, capture sequence or moiety, barcode) onto the fragments (e.g., with the use of adapter-specific primers), or mRNA or cDNA gene expression product sequences are hybridized directly to a cDNA probe comprising the additional sequence (e.g., adapter, reporter, capture sequence or moiety, barcode).

During amplification with the adapter-specific primers, a detectable label, e.g., a fluorophore, may also be added to single strand cDNA molecules.

Amplification therefore may also serve to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature, and (iv) the chemical addition of a detectable label to the cDNA molecules. In an example, the expression of a gene expression product of interest may be detected at the nucleic acid level via detection of non-natural cDNA molecules.

The gene expression products described herein may include RNA comprising the entire or partial sequence of any of the nucleic acid sequences of interest, or their non-natural cDNA product, obtained synthetically in vitro in a reverse transcription reaction. The term “fragment” may be used to refer to a portion of the polynucleotide that generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full length gene expression product polynucleotide disclosed herein. A fragment of a gene expression product polynucleotide may generally encode at least 15, 25, 30, 50, 100, 150, 200, or 250 contiguous amino acids, or up to the total number of amino acids present in a full-length gene expression product protein of the genes described herein.

In certain aspects, a gene expression profile may be obtained by whole transcriptome shotgun sequencing (“WTSS” or “RNAseq”; see, e.g., Ryan el. al. BioTechniques 45: 81-94), which makes the use of high-throughput sequencing technologies to sequence cDNA in order to about information about a sample's RNA content. In general terms, cDNA is made from RNA, the cDNA is amplified, and the amplification products are sequenced.

After amplification, the cDNA may be sequenced using any convenient method. For example, the fragments may be sequenced using Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure (Science 2005 309: 1728); Imelfort et. al. (Brief Bioinform. 2009 10:609-18); Fox el. al. (Methods Mol Biol. 2009; 553:79-108); Appleby et. al. (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. Forward and reverse sequencing primer sites that compatible with a selected next generation sequencing platform may be added to the ends of the fragments during the amplification step.

Products may be sequenced using nanopore sequencing (e.g. as described in Soni et. al. Clin Chem 53: 1996-2001, (2007), or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in each one of U.S. Pat. Nos. 5,795,782, 6,015,714, 6,627,067, 7,238,485 and 7,258,838 and U.S. patent application publications US2006003171 and US20090029477 are herein incorporated by reference in its entirety.

Products may be sequenced using Nanostring sequencing, e.g., as described in Geiss et. al. Nature Biotechnology 2007, 26(3): 317-325 or as described by NanoString Technologies). Nanostring sequencing and the like may comprise an amplification-free assay that measures nucleic acid content by counting molecules directly. Nucleic acid samples may be processed on a Nanostring instrument comprising a sequencing card and a flow cell surface. Specific capture probe pairs may be hybridized to fragmented DNA or RNA molecules from nucleic acid sample material. These captured nucleic acid molecules, with a sequencing window of up to 100 bp, may undergo sample processing, during which the core captured targets may be purified and pooled. Purified and pooled targets may then be transferred to a sequencing card where they are hybridized to the flow cell surface. Sequencing may be accomplished through multiple sequencing cycles which involve cyclic nucleic acid hybridization of targets with sequencing probes, followed by readout with reporter probes. Sequencing probes may contain a hexamer sequencing domain and a reporter domain, where sequencing domain forms the complement to the target to be sequenced, and the reporter domain may be a cyclically-read barcode. The reporter domain encoding the identity of the hexamer sequence hybridized to the target may be read via hybridization with fluorescently labeled reporter probes. Hexamer sequences derived from each single target molecule may be assembled using a graph-based algorithm and the resulting contiguous sequence reads are output into an industry-standard data output file (BAM or CRAM) that includes sequence quality metrics. Nanostring sequencing technology is disclosed in U.S. Pat. Nos. 9,381,563, 7,941,279, 8,415,102, 9,376,712, 9,856,519, 10,077,466, and U.S. patent application publication No. US20180346972, each of which is incorporated herein by reference in its entirety.

The gene expression product of the subject methods may be a protein, and the amount of protein in a particular biological sample may be analyzed using a classifier derived from protein data obtained from cohorts of samples. The amount of protein may be determined by one or more of the following: enzyme-linked immunosorbent assay (ELISA), mass spectrometry, blotting, or immunohistochemistry.

Gene expression product markers and alternative splicing markers may be determined by microarray analysis using, for example, Affymetrix arrays, cDNA microarrays, oligonucleotide microarrays, spotted microarrays, or other microarray products from Biorad, Agilent, or Eppendorf. Microarrays may contain a large number of genes or alternative splice variants that may be assayed in a single experiment. In some cases, the microarray device may contain the entire human genome or transcriptome or a substantial fraction thereof allowing a comprehensive evaluation of gene expression patterns, genomic sequence, or alternative splicing. Markers may be found using standard molecular biology and microarray analysis techniques as described in Sambrook Molecular Cloning a Laboratory Manual 2001 and Baldi, P., and Hatfield, W. G., DNA Microarrays and Gene Expression 2002.

Microarray analysis may begin with extracting and purifying nucleic acid from a biological sample, (e.g. a biopsy or fine needle aspirate). For expression and alternative splicing analysis it may be advantageous to extract and/or purify RNA from DNA. It may further be advantageous to extract and/or purify niRNA from other forms of RNA such as tRNA and rRNA.

Purified nucleic acid may further be labeled with a fluorescent label, radionuclide, or chemical label such as biotin, digoxigenin, or digoxin for example by reverse transcription, polymerase chain reaction (PGR), ligation, chemical reaction or other techniques. The labeling may be direct or indirect which may further require a coupling stage. The coupling stage can occur before hybridization, for example, using ammoallyl-UTP and NHS amino-reactive dyes (like cyanine dyes) or after, for example, using biotin and labelled streptavidin. In one example, modified nucleotides (e.g. at a 1 aaUTP: 4 TTP ratio) may be added enzymatically at a lower rate compared to normal nucleotides, typically resulting in 1 every 60 bases (measured with a spectrophotometer). The aaDNA may then be purified with, for example, a column or a diafiltration device. The aminoallyl group is an amine group on a long linker attached to the nucleobase, which reacts with a reactive label (e.g. a fluorescent dye).

The labeled samples may then be mixed with a hybridization solution which may contain sodium dodecyl sulfate (SDS), SSC, dextran sulfate, a blocking agent (such as COT1 DNA, salmon sperm DNA, calf thymus DNA, PolyA or PolyT), Denhardt's solution, formamine, or a combination thereof.

A hybridization probe may be a fragment of nucleic acid, e.g., DNA or RNA of variable length, which may be used to detect in DNA or RNA samples the presence of nucleotide sequences (the DNA target) that are complementary to the sequence in the probe. The labeled probe may be first denatured (by heating or under alkaline conditions) into single DNA strands and then hybridized to the target DNA.

To detect hybridization of the probe to its target sequence, the probe may be tagged (or labeled) with a molecular marker; commonly used markers are 32P or Digoxigenin, which is nonradioactive antibody-based marker. DNA sequences or RNA transcripts that have moderate to high sequence complementarity (e.g. at least 70%, 80%, 90%, 95%, 96%, 97%, 98%, 99%, or more complementarity) to the probe may then be detected by visualizing the hybridized probe via autoradiography or other imaging techniques. Detection of sequences with moderate or high complementarity may depend on how stringent the hybridization conditions were applied; high stringency, such as high hybridization temperature and low salt in hybridization buffers, may permit only hybridization between nucleic acid sequences that are highly similar, whereas low stringency, such as lower temperature and high salt, may allow hybridization when the sequences are less similar. Hybridization probes used in DNA microarrays may refer to DNA covalently attached to an inert surface, such as coated glass slides or gene chips, and to which a mobile cDNA target is hybridized.

A mix comprising target nucleic acid to be hybridized to probes on an array may be denatured by heat or chemical means and added to a port in a microarray. The holes may then be sealed and the microarray hybridized, for example, in a hybridization oven, where the microarray is mixed by rotation, or in a mixer. After an overnight hybridization, non-specific binding may be washed off (e.g. with SDS and SSC). The microarray may then be dried and scanned in a machine comprising a laser that excites the dye and a detector that measures emission by the dye. The image may be overlaid with a template grid and the intensities of the features (e.g. a feature comprising several pixels) may be quantified.

Various kits may be used for the amplification of nucleic acid and probe generation of the subject methods. Examples of kit that may be used in the present disclosure include but are not limited to NuGen WT-Ovation FFPE kit, cDNA amplification kit with Nugen Exon Module and Frag/Label module. The NuGEN WT-Ovation™. FFPE System V2 is a whole transcriptome amplification system that enables conducting global gene expression analysis on the vast archives of small and degraded RNA derived from FFPE samples. The system is comprised of reagents and a protocol required for amplification of as little as 50 ng of total FFPE RNA. The protocol may be used for qPCR, sample archiving, fragmentation, and labeling. The amplified cDNA may be fragmented and labeled in less than two hours for GeneChip™. 3′ expression array analysis using NuGEN's FL-Ovation™. cDNA Biotin Module V2. For analysis using Affymetrix GeneChip™ Exon and Gene ST arrays, the amplified cDNA may be used with the WT-Ovation Exon Module, then fragmented and labeled using the FL-Ovation™. cDNA Biotin Module V2. For analysis on Agilent arrays, the amplified cDNA may be fragmented and labeled using NuGEN's FL-Ovation™ cDNA Fluorescent Module.

Ambion WT-expression kit may be used for the amplification of nucleic acid and probe generation. Ambion WT-expression kit allows amplification of total RNA directly without a separate ribosomal RNA (rRNA) depletion step. With the Ambion™ WT Expression Kit, samples as small as 50 ng of total RNA may be analyzed on Affymetrix™, GeneChip™ Human, Mouse, and Rat Exon and Gene 1.0 ST Arrays. In addition to the lower input RNA requirement and high concordance between the Affymetrix™ method and TaqMan™ real-time PCR data, the Ambion™ WT Expression Kit may provide a significant increase in sensitivity. For example, a greater number of probe sets detected above background may be obtained at the exon level with the Ambion™ WT Expression Kit as a result of an increased signal-to-noise ratio. Ambion™ expression kit may be used in combination with additional Affymetrix labeling kit. For example, AmpTec Trinucleotide Nano mRNA Amplification kit (6299-A15) may be used in the subject methods. The ExpressArt™ TRinucleotide mRNA amplification Nano kit is suitable for a wide range, from 1 ng to 700 ng of input total RNA. According to the amount of input total RNA and the required yields of RNA, it may be used for 1-round (input >300 ng total RNA) or 2-rounds (minimal input amount 1 ng total RNA), with RNA yields in the range of >10 μg. AmpTec's proprietary TRinucleotide priming technology results in preferential amplification of mRNAs (independent of the universal eukaryotic 3′-poly(A)-sequence), combined with selection against rRNAs. More information on AmpTec Trinucleotide Nao mRNA Amplification kit may be obtained at www.amp-tec.com/products.htm. This kit may be used in combination with cDNA conversion kit and Affymetrix labeling kit.

### Trained Algorithm

The above described methods may be used for determining transcript expression levels for training (e.g., using a classifier training module) a classifier to differentiate whether a subject is a smoker or non-smoker. In another example, the above described methods may be used for determining transcript expression levels for training (e.g., using a classifier training module) a classifier to differentiate whether a subject has cancer or no cancer, e.g., based upon such expression levels in a sample comprising cells harvested from a nasal epithelial cell sample. In an instance, the above described methods may be used for determining transcript expression levels for training (e.g., using a classifier training module) a classifier to differentiate a subject's risk of malignancy based on transcripts of a sample obtained from the subject, e.g., based upon such expression levels in a sample comprising cells harvested from a nasal epithelial cell sample.

The trained algorithm of the present disclosure can be trained using a set of samples, such as a sample cohort. The sample cohort can comprise about 5, 10, 20, 30, 40, 50, 60, 70, 80, 90, 100, 150, 200, 250, 300, 350, 400, 450, 500, 600, 700, 800, 900, 1000, 2000, 3000, 4000, 5000 or more independent samples. The sample cohort can comprise about 100 independent samples. The sample cohort can comprise about 200 independent samples. The sample cohort can comprise between about 100 and about 700 independent samples. The independent samples can be from subjects having been diagnosed with a disease, such as cancer, from healthy subjects, or any combination thereof.

The sample cohort can comprise samples from about 5, 10, 20, 30, 40, 50, 60, 70, 80, 90, 100, 150, 200, 250, 300, 350, 400, 450, 500, 600, 700, 800, 900, 1000 or more different individuals. The sample cohort can comprise samples from about 100 different individuals. The sample cohort can comprise samples from about 200 different individuals. The different individuals can be individuals having been diagnosed with a disease, such as cancer, health individuals, or any combination thereof.

The sample cohort can comprise samples obtained from individuals living in at least 1, 2, 3, 4, 5, 6, 67, 8, 9, 10, 15, 20, 25, 30, 35, 40, 45, 50, 55, 60, 65, 70, 75, or 80 different geographical locations (e.g., sites spread out across a nation, such as the United States, across a continent, or across the world). Geographical locations may include, but are not limited to, test centers, medical facilities, medical offices, post office addresses, cities, counties, states, nations, or continents. In some cases, a classifier that is trained using sample cohorts from the United States may need to be re-trained for use on sample cohorts from other geographical regions (e.g., India, Asia, Europe, Africa, etc.).

The trained algorithm may comprise one or more classifiers. For example, the trained algorithm may comprise a lung cancer classifier, a smoking status classifier, one or more clinical classifiers, one or more genomic classifiers, or both genomic and clinic classifiers. The trained algorithm may comprise an ensemble classifier which comprises multiple independent classifiers. In an example, the trained algorithm may analyze the expression information of expression products of at least 1, 2, 3, 4, 5, 6, 7, 8, 9, 10, 10-15, 15-20, 20-22, of the genes as listed in Table 1. The trained algorithm may be used to analyze the expression information of expression products of at least 1, 2, 3, 4, 5, 6, 7, 8, 9, 10, 11, 12, 13, 14, 15, 16, 17, 18, 19, 20, 21, 22, 23, 24, 25, or 26 genes as listed in Table 3. The trained algorithm may be used to analyze the expression of expression products of at least 1, 2, 3, 4, 5, 6, 7, 8, 9, 10, 10-15, 15-20, 20-30, 30-40, 40-50, 50-60, 60-70, 70-80, 80-90, 90-100, or at least 10, at least 20, at least 30, at least 40 at least 50, at least 60, at least 70, at least 80, at least 90, at least 100, at least 110, at least 120, at least 130, at least 140, at least 150, at least 160, at least or at maximum of 170, at least or at maximum of 180, at least or at maximum of 190, at least or at maximum of 200, 210, 220, 230, 240, or 248 genes as listed in Table 2.

The method and trained algorithm described herein generally have high sensitivity. For example, the specificity of the present method is at least 60%, 65%, 70%, 75%, 80%, 85%, 86%, 87%, 88%, 89%, 90%, 91%, 92%, 93%, 94%, 95%, 96%, 97%, 98%, 99%, or more; at least 70%, 75%, 80%, 85%, 86%, 87%, 88%, 89%, 90%, 91%, 92%, 93%, 94%, 95%, 96%, 97%, 98%, 99%, or more; or at least greater than or equal to 60%.

In certain instances, the negative predictive value (NPV) of a biological sample analyzed by a classifier may be greater than or equal to 80%. The NPV may be at least 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% or more.

Sensitivity typically refers to TP/(TP+FN), where TP is true positive and FN is false negative. Number of Continued Indeterminate results divided by the total number of malignant results based on adjudicated histopathology diagnosis. Specificity typically refers to TN/(TN+FP), where TN is true negative and FP is false positive. The number of actual benign results is divided by the total number of benign results based on adjudicated histopathology diagnosis. Positive Predictive Value (PPV) may be determined by: TP/(TP+FP). Negative Predictive Value (NPV) may be determined by TN/(TN+FN).

A biological sample may be identified as cancerous with an accuracy of greater than 75%, 80%, 85%, 90%, 95%, 99% or more. For example, the biological sample may be identified as cancerous with a sensitivity of greater than 90%. In another example, the biological sample may be identified as cancerous with a specificity of greater than 60%. The biological sample identified as cancerous or benign may have a sensitivity of greater than 90% and a specificity of greater than 60%. The accuracy or sensitivity may be calculated using a trained algorithm.

Results of the expression analysis of the subject methods may provide a statistical confidence level that a given diagnosis is correct. Such statistical confidence level may be above 85%, 90%, 91%, 92%, 93%, 94%, 95%, 96%, 97%, 98%, 99% or 99.5%.

A trained algorithm may produce a unique output each time it is run. For example, using a different sample or plurality of samples with the same classifier can produce a unique output each time the classifier is run. Using the same sample or plurality of samples with the same classifier can produce a unique output each time the classifier is run. Using the same samples to train a classifier more than one time may result in unique outputs each time the classifier is run.

Characteristics of a sample (e.g., mRNA expression levels) can be analyzed using an algorithm that comprises one or more classifiers and which is trained using one or more an annotated reference sets. The identification can be performed by the classifier. More than one characteristic of a sample can be combined to generate classification of tissue sample. In some cases, gene expression levels of one or more genes from a sample can be processed relative to expression levels of a reference set of genes that are used to train one or more classifiers to determine the presence of differential gene expression of one or more genes. A reference set can comprise one or more housekeeping genes. The reference set can comprise known sequence variants or expression levels of genes known to be associated with a particular disease or known to be associated with a non-disease state.

Classifiers of a trained algorithm can perform processing, combining, statistical evaluation, or further analysis of results, or any combination thereof. Performance of any of the forgoing may be automated by a computer system. Separate reference sets may be provided for different features. For example, sequence variant data may be processed relative to a sequence variant data reference set. A gene expression level data may be processed relative to a gene expression level reference set. In some cases, multiple feature spaces may be processed with respect to the same reference set.

Data from the methods described, such as gene expression levels can be further analyzed using feature selection techniques such as filters which can assess the relevance of specific features by looking at the intrinsic properties of the data, wrappers which embed the model hypothesis within a feature subset search, or embedded protocols in which the search for an optimal set of features is built into a classifier algorithm.

Filters useful in the methods of the present disclosure can include, for example, (1) parametric methods such as the use of two sample t-tests, analysis of variance (ANOVA) analyses, Bayesian frameworks, or Gamma distribution models (2) model free methods such as the use of Wilcoxon rank sum tests, between-within class sum of squares tests, rank products methods, random permutation methods, or threshold number of misclassification (TNoM) which involves setting a threshold point for fold-change differences in expression between two datasets and then detecting the threshold point in each gene that minimizes the number of mis-classifications or (3) multivariate methods such as bivariate methods, correlation based feature selection methods (CFS), minimum redundancy maximum relevance methods (MRMR), Markov blanket filter methods, and uncorrelated shrunken centroid methods. Wrappers useful in the methods of the present disclosure can include sequential search methods, genetic algorithms, or estimation of distribution algorithms. Embedded protocols can include random forest algorithms, weight vector of support vector machine algorithms, or weights of logistic regression algorithms.

Raw data obtained from expression profile analyses may be normalized. Normalization may be performed, for example, by subtracting the background intensity and then dividing the intensities making either the total intensity of the features on each channel equal or the intensities of a reference gene and then the t-value for all the intensities may be calculated. More sophisticated methods include z-ratio, loess and lowess regression and RMA (robust multichip analysis), such as for Affymetrix chips.

Statistical evaluation of the results obtained from the methods described herein can provide a quantitative value or values indicative of one or more of the following: the classification of the tissue sample; the likelihood of diagnostic accuracy; the likelihood of disease, such as cancer; and the likelihood of the success of a particular therapeutic intervention. Thus a medical professional, who may not be trained in genetics or molecular biology, need not understand gene expression level or sequence variant data results. Rather, data can be presented directly to the medical professional in its most useful form to guide care or treatment of the subject. Statistical evaluation, combination of separate data results, and reporting useful results can be performed by the trained algorithm. Statistical evaluation of results can be performed using a number of methods including, but not limited to: the students T test, the two sided T test, pearson rank sum analysis, hidden markov model analysis, analysis of q-q plots, principal component analysis, one way analysis of variance (ANOVA), two way ANOVA, and the like. Statistical evaluation can be performed by the trained algorithm.

The presently described gene expression profile can also be used to screen for subjects who are susceptible to or otherwise at risk for developing lung cancer. For example, a current smoker of advanced age (e.g., 70 years old) may be at an increased risk for developing lung cancer and may represent an ideal candidate for the assays and methods disclosed herein. Moreover, the early detection of lung cancer in such a subject may improve the subject's overall survival. Accordingly, in certain aspects, the assays and methods disclosed herein are performed or otherwise comprise an analysis of the subject's clinical risk factors for developing cancer. For example, one or more clinical risk factors selected from the group consisting of advanced age (e.g., age greater than about 40 years, 50 years, 55 years, 60 years, 65 years, 70 years, 75 years, 80 years, 85 years, 90 years or more), smoking status, the presence of a lung nodule greater than 3 cm on CT scan, the lesion or nodule location (e.g., centrally located, peripherally located or both) and the time since the subject quit smoking. The assays and methods disclosed herein may further comprise a step of considering the presence of any such clinical risk factors to inform the determination of whether the subject has lung cancer or is at risk of developing lung cancer.

In certain aspects, the methods and assays disclosed herein may be useful for determining a treatment course for a subject. For example, such methods and assays may involve determining the expression levels of one or more genes (e.g., one or more of the genes set forth in Table 2 or Table 3) in a biological sample obtained from the subject, and determining a treatment course for the subject based on the expression profile of such one or more genes. The treatment course may be determined based on a lung cancer risk-score derived from the expression levels of the one or more genes analyzed. The subject may be identified as a candidate for a lung cancer therapy based on an expression profile that indicates the subject has a relatively high risk of malignancy for lung cancer. The subject may be identified as a candidate for an invasive lung procedure (e.g., transthoracic needle aspiration, mediastinoscopy, lobectomy, or thoracotomy) based on an expression profile that indicates the subject has a relatively high risk of malignancy for lung cancer (e.g., greater than 60%, greater than 70%, greater than 80%, greater than 90%). A relatively high risk of malignancy may mean greater than about a 60% chance of having lung cancer. In certain aspects, a relatively high risk of malignancy means greater than about a 75% chance of having lung cancer. In certain aspects, a relatively high risk of malignancy means greater than about an 80-85% chance of having lung cancer. In certain aspects, a very high risk of malignancy means greater than about a 90% chance of having lung cancer. In one example, relatively low risk of malignancy means less than 10% chance of having lung cancer.

A trained algorithm as provided herein can be used to further up- or down-classify a sample of a subject with intermediate risk of malignancy, corresponding to an inconclusive pre-test malignancy (e.g., the first level of risk of malignancy). A second level of risk of malignancy for a sample obtained from a subject may be generated based on a first level of risk of malignancy and one or more genomic features and one or more clinical features. The second level of risk of malignancy may be an up- or down-classification of the first level of risk of malignancy. The first level of risk of malignancy may be determined using clinical risk factors, for example. This may be re-classified upon analyzing one or more clinical features and one or more genomic features from a subject's sample using a trained algorithm. For example, a subject with a pre-test low risk of malignancy for lung cancer (e.g., less than 10%) may be re-classified as having very low risk of having lung cancer (less than 1%) with an NPV no less than 99%. This may be based on one or more genomic features that include expression of one or more genes as listed in Table 1 or Table 3 or Table 37. A subject with a pre-test intermediate risk of malignancy (e.g., 10-60%) for lung cancer may be re-classified as having low risk (e.g., less than 10%) of malignancy for having lung cancer with an NPV no less than 91%. This may be based on one or more genomic features that include expression of one or more genes as listed in Table 1 or Table 3 or Table 37. In another example, a subject with a pre-test intermediate risk of malignancy of lung cancer may be re-classified as having high risk (e.g., greated than 60%) of having lung cancer with an PPV no less than 65%. They may be based on one or more genomic features that include expression of one or more genes as listed in Table 1 or Table 3 or Table 37. In yet another example, a subject with a pre-test high risk of malignancy (e.g., greater than 60%) of having lung cancer may be re-classified as having very high risk of malignancy (e.g., greater than 90%) for having lung cancer with an PPV no less than 91%. This may be based on one or more genomic features that include expression of one or more genes as listed in Table 1 or Table 3 or Table 37. Accordingly, in certain aspects of the present disclosure, if the methods disclosed herein are indicative of the subject having lung cancer or of being at risk of developing lung cancer, such methods may comprise additionally treating the subject (e.g., administering to the subject a treatment comprising one or more of chemotherapy, radiation therapy, immunotherapy, surgical intervention and combinations thereof).

In the methods of the present disclosure, a subject may be monitored. For example, a subject may be diagnosed with cancer. This initial diagnosis may or may not involve the use of methods disclosed herein. The subject may be prescribed a therapeutic intervention such as a thyroidectomy for a subject suspected of having lung cancer. The results of the therapeutic intervention may be monitored on an ongoing basis by methods disclosed herein to detect the efficacy of the therapeutic intervention. In another example, a subject may be diagnosed with a benign tumor or a precancerous lesion or nodule, and the tumor, nodule, or lesion may be monitored on an ongoing basis by methods disclosed herein to detect any changes in the state of the tumor or lesion. In another aspect, a subject may be diagnosed with a non-conclusive likelihood of having or developing lung cancer. If the methods and assays disclosed herein are indicative of a subject being at a high or very high risk of having or developing lung cancer, the subject may be subjected to more invasive monitoring, such as a direct tissue sampling or biopsy of the nodule, under the presumption that the positive test indicates a higher likelihood of the nodule is a cancer. On the basis of the methods and assays disclosed herein being indicative of a subject's higher risk of having or developing lung cancer, an appropriate therapeutic regimen (e.g., chemotherapy or radiation therapy) may be administered to the subject. Subjects having a low or very low risk of developing lung cancer is may be subjected to further confirmatory testing, such as further imaging surveillance (e.g., a repeat CT scan to monitor whether the nodule grows or changes in appearance before doing a more invasive procedure), or a determination made to withhold a particular treatment (e.g., chemotherapy or radiation therapy) on the basis of the subject's favorable or reduced risk of having or developing lung cancer. The assays and methods disclosed herein may be used to confirm the results or findings from a more invasive procedure, such as direct tissue sampling or biopsy. For example, in certain aspects the assays and methods disclosed herein may be used to confirm or monitor the benign status of a previously biopsied nodule or lesion.

The methods and assays disclosed herein may be useful for determining a treatment course for a subject that has undergone an indeterminate or nondiagnostic bronchoscopy does not have lung cancer, wherein the method comprises determining the expression levels of one or more genes (e.g., one or more of the genes set forth in Table 1 or Table 3 or Table 37) in a sample of cells, e.g. nasal epithelial cells obtained from the subject, and determining whether the subject that has undergone an indeterminate or non-diagnostic bronchoscopy does or does not have lung cancer or is not at risk of developing lung cancer. The methods and assays described herein may comprise determining a lung cancer risk-score derived from the expression levels of the one or more genes analyzed. In an example, the subject that has undergone an indeterminate or non-diagnostic bronchoscopy would have typically been identified as being a candidate for an invasive lung procedure (e.g., transthoracic needle aspiration, mediastinoscopy, lobectomy, or thoracotomy) based upon such indeterminate of nondiagnostic bronchoscopy result, but the subject may be instead identified as being a candidate for a non-invasive procedure (e.g., monitoring by CT scan) because the subjects expression levels of the one or more genes (e.g., one or more of the genes set forth in Table 1 or Table 3 or Table 37) in the sample of cells, e.g. nasal epithelial cells obtained from the subject indicates that the subject has a low risk of having lung cancer (e.g. the instant method indicates that the subject has less than 10%, less than 5%, or less than 1% chance of having cancer). In an example, the subject may be identified as a candidate for an invasive lung cancer therapy based on an expression profile that indicates the subject has a relatively high risk of malignancy (e.g., where the instant method indicates that the subject has a greater than 60% chance of having cancer, or a greater than 70%, 80%, or greater than 90% chance of having cancer). Accordingly, in certain aspects of the present disclosure, if the methods disclosed herein are indicative of the subject having lung cancer or of being at risk of developing lung cancer, such methods may comprise a further step of treating the subject (e.g., administering to the subject a treatment comprising one or more of chemotherapy, radiation therapy, immunotherapy, surgical intervention and combinations thereof).

In some cases, an expression profile is obtained and the subject may not be indicated as being in the high risk or the low risk categories. For example, a health care provider may elect to monitor the subject and repeat the assays or methods at one or more later points in time, or undertake further diagnostics procedures to rule out lung cancer, or make a determination that cancer is present, soon after the subject's lung cancer risk determination was made.

In some aspects, the present disclosure relates to compositions that may be used to determine the expression profile of one or more genes from a subject's biological sample comprising nasal epithelial cells. For example, compositions are provided may comprise nucleic acid probes that specifically hybridize with one or more genes set forth in Table 1, Table 2 or Table 3. These compositions may also include probes that specifically hybridize with one or more control genes and may further comprise appropriate buffers, salts or detection reagents. Such probes may be fixed directly or indirectly to a solid support (e.g., a glass, plastic or silicon chip) or a bead (e.g., a magnetic bead).

The compositions described herein may be assembled into diagnostic or research kits to facilitate their use in one or more diagnostic or research applications. In some embodiments, such kits and diagnostic compositions may be provided that comprise one or more probes capable of specifically hybridizing to at least 1, 2, 3, 4, 5, 6, 7, 8, 9, 10, 10-15, 15-20, 20-30, 30-40, 40-50, 50-60, 60-70, 70-80, 80-90, 90-100, or at least 10, at least 20, at least 30, at least 40 at least 50, at least 60, at least 70, at least 80, at least 90, at least 100, at least 110, at least 120, at least 130, at least 140, at least 150, at least 160, at least or at maximum of 170, at least or at maximum of 180, at least or at maximum of 190 of the genes as listed in Table 1. The kits and diagnostic compositions may comprise one or more probes capable of specifically hybridizing to at least 1, 2, 3, 4, 5, 6, 7, 8, 9, 10, 11, 12, 13, 14, 15, 16, 17, 18, 19, 20, 21, 22, 23, 24, 25, or 26 genes as listed in Table 3. In an example, the kits and diagnostic compositions may comprise one or more probes capable of specifically hybridizing to at least 1, 2, 3, 4, 5, 6, 7, 8, 9, 10, 10-15, 15-20, 20-30, 30-40, 40-50, 50-60, 60-70, 70-80, 80-90, 90-100, or at least 10, at least 20, at least 30, at least 40 at least 50, at least 60, at least 70, at least 80, at least 90, at least 100, at least 110, at least 120, at least 130, at least 140, at least 150, at least 160, at least or at maximum of 170, at least or at maximum of 180, at least or at maximum of 190, at least or at maximum of 200, 210, 220, 230, 240, or 248 genes as listed in Table 2.

A kit may include one or more containers housing one or more of the components provided in this disclosure and instructions for use. Specifically, such kits may include one or more compositions described herein, along with instructions describing the intended application and the proper use and/or disposition of these compositions. Kits may contain the components in appropriate concentrations or quantities for running various experiments.

### Computer Systems

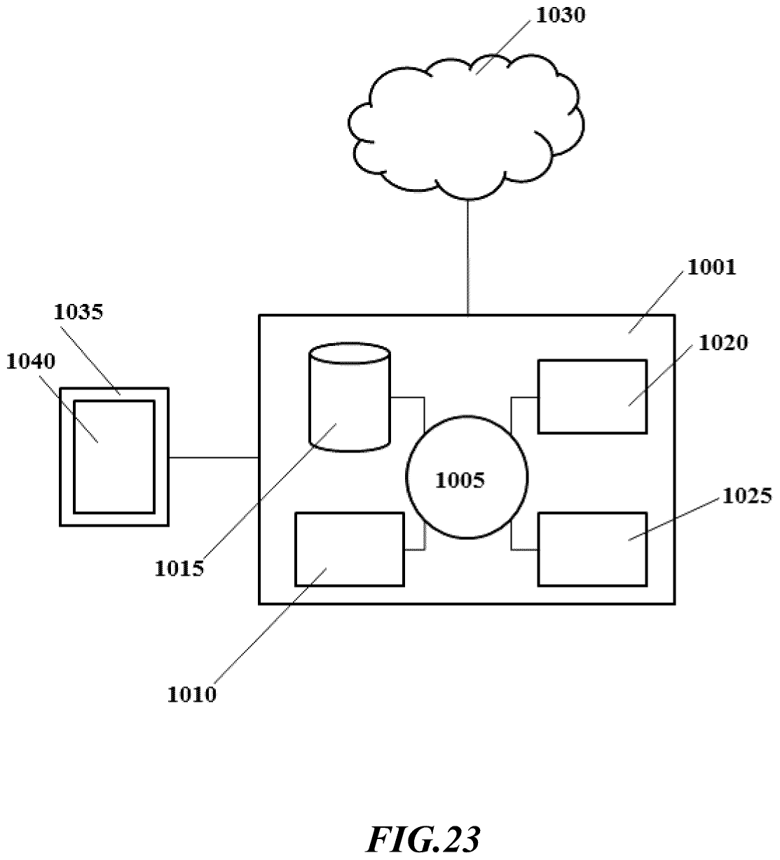

The present disclosure provides computer systems for implementing methods provided herein. FIG. 23 shows an example of a computer system 1001. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 05 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., remote cloud server). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, an electronic output of identified gene fusions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005.

### Interventions

The computer system can include or be in communication with an electronic display that comprises a user interface (UI) for providing, for example, results of nucleic acid sequencing, analysis of nucleic acid sequencing data, characterization of nucleic acid sequencing samples, tissue characterizations, etc. Examples of UI's include, without limitation, a graphical user interface (GUI) and web-based user interface. Treatment may be provided or administered to a subject based on a classification of subject's sample as positive or negative for a condition, likelihood of a condition, such as lung cancer, or risk of malignancy for a condition such as lung cancer. A treatment may be an intervention by a medical professional or in the form of providing actionable information to a subject in the form a tangible report (e.g., delivered through a computer system to be displayed to a subject on a graphical user interface, or a paper copy of a report).

An intervention by a medical profession may involve, by way of non-limiting examples, screening, monitoring, or administering therapy. Screening may include various imaging, or diagnostic testing techniques. Screening using imaging may include a low-dose computerized tomography (CT) scan and X-ray. In a non-limiting example, methods and systems of the present disclosure may be used after a lung nodule is identified in an imaging scan. Imaging may be used to screen or monitor a subject after he or she receives classification results. Diagnostic assays may similarly be used to identify a subject as a candidate for use of the methods of systems disclosed in the instant application. Such assays may include but are not limited to sputum cytology, tissue sample biopsy, immunoblot analysis, RNA sequencing or genome sequencing. Monitoring may involve a low-dose computerized tomography (CT) scan, X-ray, sputum cytology, RNA sequencing or genome sequencing.

In the event that a lung condition, such as cancer, is detected using the systems and methods of the instant disclosure, a therapy may be administered to a subject in need thereof. A therapy may involve, for example, the administration of one or more therapeutic agents or a surgical procedure. Non-limiting examples of therapeutic agents include chemotherapeutic agents, monoclonal antibodies, antibody drug conjugates, EGFR inhibitors, and ALK protein binding agents. A surgical procedure may involve, but is not limited to, thoracotomy, lobectomy, thoracoscopy, segmentectomy, wedge resection, or pneumonectomy. Treatment or therapy may include but is not limited to chemotherapy, radiation therapy, immunotherapy, hormone therapy, and pulmonary rehabilitation.

A treatment may be a medical intervention in the form of a report provided to a subject or to a medical professional. A medical professional may act as an intermediary and deliver results directly to a subject. The report may provide information such as the presence or absence of gene fusion(s) and results generated from classifying a sample as positive or negative for a lung condition based in part on assaying nucleic acids from epithelial cells in the subject's respiratory tract, such as lung cancer. The report may provide information regarding potential treatment options, such as potential drugs or clinical trials, based in part on the fusions detected.

By way of illustrative example, if a sample is classified as positive for lung cancer using the systems or methods of the present disclosure, then the subject may receive one or more of chemotherapy, radiation therapy, immunotherapy, hormone therapy, pulmonary rehabilitation, or any combination thereof. In another non-limiting example, if a sample is classified as negative for lung cancer using the systems or methods of the present disclosure, then the subject may be monitored on an on-going basis, for example, continuing imaging surveillance, for potential development of cancerous nodules or lesions.

Methods and systems of the present disclosure can be implemented by way of one or more algorithms. An algorithm can be implemented by way of software upon execution by the central processing unit. The algorithm can, for example, initiate nucleic acid sequencing, process nucleic acid sequencing data, interpret nucleic acid sequencing results, characterize nucleic acid samples, characterize samples, etc.

TABLE 1
Top-ranked Classifier Genes
Gene Name

SLC7A11
CLDN10
TKT
RUNX1T1
AKR1C2
RPS4Y1
BST1
CD177.1
CD177.2
ATP12A
TSPAN2
GABBR1
MCAM
NOVA1
SDC2
CDR1
CGREF1
CLDN22
NKX3-1
EPHX3
LYPD2
MIA
RNF150

TABLE 2
Smoking index genes

| Gene ID | Gene Name |
|---|---|
| ENSG00000083807 | SLC27A5 |
| ENSG00000089248 | ERP29 |
| ENSG00000105538 | RASIP1 |
| ENSG00000153823 | PID1 |
| ENSG00000166681 | NGFRAP1 |
| ENSG00000177707 | PVRL3 |
| ENSG00000166224 | SGPL1 |
| ENSG00000183840 | GPR39 |
| ENSG00000123739 | PLA2G12A |
| ENSG00000145428 | RNF175 |
| ENSG00000165632 | TAF3 |
| ENSG00000104517 | UBR5 |
| ENSG00000183943 | PRKX |
| ENSG00000211667 | IGLV3-12 |
| ENSG00000081189 | MEF2C |
| ENSG00000185842 | DNAH14 |
| ENSG00000009335 | UBE3C |
| ENSG00000145332 | KLHL8 |
| ENSG00000135100 | HNF1A |
| ENSG00000154165 | GPR15 |
| ENSG00000184845 | DRD1 |
| ENSG00000126895 | AVPR2 |
| ENSG00000198108 | CHSY3 |
| ENSG00000135298 | BAI3 |
| ENSG00000255093 | RP11-794P6.2 |
| ENSG00000105472 | CLEC11A |
| ENSG00000186160 | CYP4Z1 |
| ENSG00000170153 | RNF150 |
| ENSG00000138658 | C4orf21 |
| ENSG00000137460 | FHDC1 |
| ENSG00000102043 | MTMR8 |
| ENSG00000147010 | SH3KBP1 |
| ENSG00000152822 | GRM1 |
| ENSG00000144285 | SCN1A |
| ENSG00000180532 | ZSCAN4 |
| ENSG00000261857 | MIA |
| ENSG00000188385 | JAKMIP3 |
| ENSG00000139117 | CPNE8 |
| ENSG00000154978 | VOPP1 |
| ENSG00000156804 | FBXO32 |
| ENSG00000179673 | RPRML |
| ENSG00000214357 | NEURL1B |
| ENSG00000082293 | COL19A1 |
| ENSG00000138798 | EGF |
| ENSG00000135083 | CCNJL |
| ENSG00000255277 | ABCC6P2 |
| ENSG00000120658 | ENOX1 |
| ENSG00000177181 | RIMKLA |
| ENSG00000154975 | CA10 |
| ENSG00000136274 | NACAD |
| ENSG00000207698 | MIR32 |
| ENSG00000172551 | MUCL1 |
| ENSG00000100461 | RBM23 |
| ENSG00000269657 | AC079210.1 |
| ENSG00000176406 | RIMS2 |
| ENSG00000206532 | RP11-553A10.1 |
| ENSG00000200478 | SNORD115-41 |
| ENSG00000239149 | SNORA59A |
| ENSG00000168243 | GNG4 |
| ENSG00000073150 | PANX2 |
| ENSG00000165899 | OTOGL |
| ENSG00000063438 | AHRR |
| ENSG00000251615 | RP11-774O3.3 |
| ENSG00000167723 | TRPV3 |
| ENSG00000135778 | NTPCR |
| ENSG00000145423 | SFRP2 |
| ENSG00000110881 | ASIC1 |
| ENSG00000154277 | UCHL1 |
| ENSG00000130595 | TNNT3 |
| ENSG00000075213 | SEMA3A |
| ENSG00000134769 | DTNA |
| ENSG00000231663 | RP5-827C21.4 |
| ENSG00000067798 | NAV3 |
| ENSG00000174607 | UGT8 |
| ENSG00000075461 | CACNG4 |
| ENSG00000211804 | TRDV1 |
| ENSG00000156968 | MPV17L |
| ENSG00000115295 | CLIP4 |
| ENSG00000115902 | SLC1A4 |
| ENSG00000185442 | FAM174B |
| ENSG00000016402 | IL20RA |
| ENSG00000119711 | ALDH6A1 |
| ENSG00000139410 | SDSL |
| ENSG00000174175 | SELP |
| ENSG00000002745 | WNT16 |
| ENSG00000156869 | FRRS1 |
| ENSG00000151715 | TMEM45B |
| ENSG00000222018 | C21orf140 |
| ENSG00000170571 | EMB |
| ENSG00000186377 | CYP4X1 |
| ENSG00000227471 | AKR1B15 |
| ENSG00000204529 | GUCY2EP |
| ENSG00000123570 | RAB9B |
| ENSG00000151388 | ADAMTS12 |
| ENSG00000115353 | TACR1 |
| ENSG00000186940 | CHCHD2P9 |
| ENSG00000231752 | EMBP1 |
| ENSG00000187513 | GJA4 |
| ENSG00000162873 | KLHDC8A |
| ENSG00000162520 | SYNC |
| ENSG00000006611 | USH1C |
| ENSG00000147408 | CSGALNACT1 |
| ENSG00000169174 | PCSK9 |
| ENSG00000235169 | SMIM1 |
| ENSG00000179954 | SSC5D |
| ENSG00000204178 | TMEM57 |
| ENSG00000165731 | RET |
| ENSG00000154188 | ANGPT1 |
| ENSG00000154822 | PLCL2 |
| ENSG00000125378 | BMP4 |
| ENSG00000145349 | CAMK2D |
| ENSG00000163817 | SLC6A20 |
| ENSG00000243627 | AP000322.53 |
| ENSG00000136044 | APPL2 |
| ENSG00000196557 | CACNA1H |
| ENSG00000171044 | XKR6 |
| ENSG00000108018 | SORCS1 |
| ENSG00000255569 | TRAV1-1 |
| ENSG00000102409 | BEX4 |
| ENSG00000068796 | KIF2A |
| ENSG00000163872 | YEATS2 |
| ENSG00000254614 | AP003068.23 |
| ENSG00000201143 | SNORD115-42 |
| ENSG00000100628 | ASB2 |
| ENSG00000214841 | AC005493.1 |
| ENSG00000008196 | TFAP2B |
| ENSG00000207932 | MIR33A |
| ENSG00000115486 | GGCX |
| ENSG00000138316 | ADAMTS14 |
| ENSG00000197353 | LYPD2 |
| ENSG00000138386 | NAB1 |
| ENSG00000075673 | ATP12A |
| ENSG00000104432 | IL7 |
| ENSG00000155561 | NUP205 |
| ENSG00000005108 | THSD7A |
| ENSG00000268758 | EMR4P |
| ENSG00000112818 | MEP1A |
| ENSG00000266208 | CTD-2267D19.3 |
| ENSG00000100739 | BDKRB1 |
| ENSG00000092068 | SLC7A8 |
| ENSG00000128610 | FEZF1 |
| ENSG00000145362 | ANK2 |
| ENSG00000170549 | IRX1 |
| ENSG00000153933 | DGKE |
| ENSG00000168959 | GRM5 |
| ENSG00000232629 | HLA-DQB2 |
| ENSG00000196581 | AJAP1 |
| ENSG00000124939 | SCGB2A1 |
| ENSG00000180357 | ZNF609 |
| ENSG00000147573 | TRIM55 |
| ENSG00000236869 | RP11-944L7.4 |
| ENSG00000117154 | IGSF21 |
| ENSG00000137868 | STRA6 |
| ENSG00000129990 | SYT5 |
| ENSG00000095713 | CRTAC1 |
| ENSG00000128683 | GAD1 |
| ENSG00000180611 | MB21D2 |
| ENSG00000157445 | CACNA2D3 |
| ENSG00000170214 | ADRA1B |
| ENSG00000108878 | CACNG1 |
| ENSG00000272173 | U47924.31 |
| ENSG00000144369 | FAM171B |
| ENSG00000102174 | PHEX |
| ENSG00000146250 | PRSS35 |
| ENSG00000167210 | LOXHD1 |
| ENSG00000166582 | CENPV |
| ENSG00000073734 | ABCB11 |
| ENSG00000137968 | SLC44A5 |
| ENSG00000240694 | PNMA2 |
| ENSG00000144426 | NBEAL1 |
| ENSG00000107562 | CXCL12 |
| ENSG00000124678 | TCP11 |
| ENSG00000103175 | WFDC1 |
| ENSG00000262222 | RP11-876N24.4 |
| ENSG00000154845 | PPP4R1 |
| ENSG00000221923 | ZNF880 |
| ENSG00000134256 | CD101 |
| ENSG00000166947 | EPB42 |
| ENSG00000254461 | RP11-755F10.3 |
| ENSG00000163393 | SLC22A15 |
| ENSG00000237188 | RP11-337C18.8 |
| ENSG00000166923 | GREM1 |
| ENSG00000146013 | GFRA3 |
| ENSG00000258875 | CTD-2547L24.3 |
| ENSG00000041515 | MYO16 |
| ENSG00000197558 | SSPO |
| ENSG00000175213 | ZNF408 |
| ENSG00000204179 | PTPN20A |
| ENSG00000159648 | TEPP |
| ENSG00000081052 | COL4A4 |
| ENSG00000139173 | TMEM117 |
| ENSG00000206538 | VGLL3 |
| ENSG00000184117 | NIPSNAP1 |
| ENSG00000164796 | CSMD3 |
| ENSG00000135346 | CGA |
| ENSG00000185518 | SV2B |
| ENSG00000188738 | FSIP2 |
| ENSG00000109472 | CPE |
| ENSG00000163029 | SMC6 |
| ENSG00000101342 | TLDC2 |
| ENSG00000168785 | TSPAN5 |
| ENSG00000172572 | PDE3A |
| ENSG00000134775 | FHOD3 |
| ENSG00000166897 | ELFN2 |
| ENSG00000070159 | PTPN3 |
| ENSG00000112208 | BAG2 |
| ENSG00000184389 | A3GALT2 |
| ENSG00000074211 | PPP2R2C |
| ENSG00000207579 | MIR662 |
| ENSG00000163788 | SNRK |
| ENSG00000137198 | GMPR |
| ENSG00000147041 | SYTL5 |
| ENSG00000224361 | AC011239.1 |
| ENSG00000142528 | ZNF473 |
| ENSG00000250989 | RP11-392E22.5 |
| ENSG00000105784 | RUNDC3B |
| ENSG00000004939 | SLC4A1 |
| ENSG00000013392 | RWDD2A |
| ENSG00000173557 | C2orf70 |
| ENSG00000207562 | MIR34C |
| ENSG00000168811 | IL12A |
| ENSG00000162402 | USP24 |
| ENSG00000166123 | GPT2 |
| ENSG00000101152 | DNAJC5 |
| ENSG00000159712 | ANKRD18CP |
| ENSG00000139116 | KIF21A |
| ENSG00000224689 | ZNF812 |
| ENSG00000117501 | MROH9 |
| ENSG00000172985 | SH3RF3 |
| ENSG00000215271 | HOMEZ |
| ENSG00000254761 | RP11-672A2.1 |
| ENSG00000112812 | PRSS16 |
| ENSG00000072657 | TRHDE |
| ENSG00000176473 | WDR25 |
| ENSG00000164867 | NOS3 |
| ENSG00000244734 | HBB |
| ENSG00000263142 | LRRC37A17P |
| ENSG00000166974 | MAPRE2 |
| ENSG00000179914 | ITLN1 |
| ENSG00000076864 | RAP1GAP |
| ENSG00000198467 | TPM2 |
| ENSG00000126091 | ST3GAL3 |
| ENSG00000184347 | SLIT3 |
| ENSG00000128596 | CCDC136 |
| ENSG00000117479 | SLC19A2 |
| ENSG00000171403 | KRT9 |
| ENSG00000207728 | MIR449B |
| ENSG00000110777 | POU2AF1 |

TABLE 3
Nasal classifier genes related to lung cancer

| Gene ID | Gene Name |
|---|---|
| ENSG00000119946 | CNNM1 |
| ENSG00000143507 | DUSP10 |
| ENSG00000166289 | PLEKHF1 |
| ENSG00000052344 | PRSS8 |
| ENSG00000102878 | HSF4 |
| ENSG00000179933 | C14orf119 |
| ENSG00000142173 | COL6A2 |
| ENSG00000136379 | ABHD17C |
| ENSG00000147883 | CDKN2B |
| ENSG00000034677 | RNF19A |
| ENSG00000204262 | COL5A2 |
| ENSG00000198492 | YTHDF2 |
| ENSG00000121858 | TNFSF10 |
| ENSG00000134339 | SAA2 |
| ENSG00000120875 | DUSP4 |
| ENSG00000131979 | GCH1 |
| ENSG00000106351 | AGFG2 |
| ENSG00000103342 | GSPT1 |
| ENSG00000204576 | PRR3 |
| ENSG00000140750 | ARHGAP17 |
| ENSG00000070159 | PTPN3 |
| ENSG00000115641 | FHL2 |
| ENSG00000071575 | TRIB2 |
| ENSG00000112769 | LAMA4 |
| ENSG00000170791 | CHCHD7 |
| ENSG00000050405 | LIMA1 |

### EXAMPLES

### Example 1: Development of an Algorithm to Determine Smoking Status by Gene Expression from Lung Bronchial Epithelial Tissue

Over 1500 samples from three separate patient cohorts were used to develop and test the method. The three patient cohorts are Aegis I and Aegis II, the Percepta Registry, and DECAMP-1.

Aegis I and Aegis II include samples from patients with suspicious nodules detected on CT and who underwent bronchoscopy. A large proportion of the patients have diagnostic bronchoscopy. A large proportion of the patients have a high pre-test risk of malignancy (both diagnostic and nondiagnostic bronchoscopy groups). Follow up is one year.

The Percepta Registry includes an observational study designed to evaluate Percepta usage in a real-world setting. Non-diagnostic bronchoscopies only, the majority of samples are composed of samples with an intermediate pre-test risk of malignancy. Follow up is one year.

DECAMP-1, “Detection of Early Lung Cancer Among Military Personnel Study 1 (DECAMP-1): Diagnosis and Surveillance of Intermediate Pulmonary Nodules” is enriched with veterans. Cancer prevalence in the pre-test intermediate non-diagnostic bronchoscopy group is 50.8%. Follow up is 2 years.

The samples used to train the classifier are identified in Table 13 below:

TABLE 13
Representative samples used in training classifiers.

| | | Sample type (in training) | | | |
|---|---|---|---|---|---|
| Classifier | Cohort | OOI | Primary | Prior cancer | Total |
| M2 | AEGIS | 579 | 189 | — | 768 |
| | DECAMP1 | 41 | — | — | 41 |
| | Registry | — | 122 | — | 122 |
| | Total | 620 | 311 | — | 931 |
| Smoking | AEGIS | 894 | 189 | 123 | 1206 |
| index | DECAMP1 | 119 | — | 21 | 140 |
| | Registry | 52 | 122 | 58 | 232 |
| | Total | 1065 | 311 | 202 | 1578 |
| Collecting | Registry | 85 | 122 | 58 | 265 |
| timing | Total | 85 | 122 | 58 | 265 |
| All 3 | AEGIS | 894 | 189 | 123 | 1206 |
| classifiers | DECAMP1 | 119 | — | 21 | 140 |
| combined | Registry | 85 | 122 | 58 | 265 |
| | Total | 1065 | 311 | 202 | 1611 |

Next generation sequencing of the purified RNA was carrier out to measure expression of coding RNA. The resulting gene list was curated to remove those gene associated with technical factors. A final set of 17,782 genes was then analyzed using the machine learning algorithms svm and glmnet in a cross-validation system (as can be seen in Table 20 below). RNA-seq data was used to generate gene expression counts.

TABLE 20
Representative data from bronchial samples indicating
that different combinations of models and input
genes can give an AUC greater than 0.95.

| | num Genes in | |
|---|---|---|
| modName | FeatureSet FullModel | median_CVAUC |
| byPvalue-glmnet | 248 | 0.956 |
| byPvalue-svm | 12273 | 0.954 |
| hcProp0.1-glmnet | 124 | 0.951 |
| hcProp0.1-svm | 426 | 0.952 |
| hcProp0.2-glmnet | 125 | 0.951 |
| hcProp0.2-svm | 491 | 0.952 |
| hcProp0.5-glmnet | 130 | 0.955 |
| hcProp0.5-svm | 965 | 0.952 |
| hpProp0.1-glmnet | 73 | 0.952 |
| hpProp0.1-svm | 195 | 0.952 |
| hpProp0.2-glmnet | 92 | 0.954 |
| hpProp0.2-svm | 396 | 0.953 |
| hpProp0.5-glmnet | 130 | 0.955 |
| hpProp0.5-svm | 997 | 0.953 |

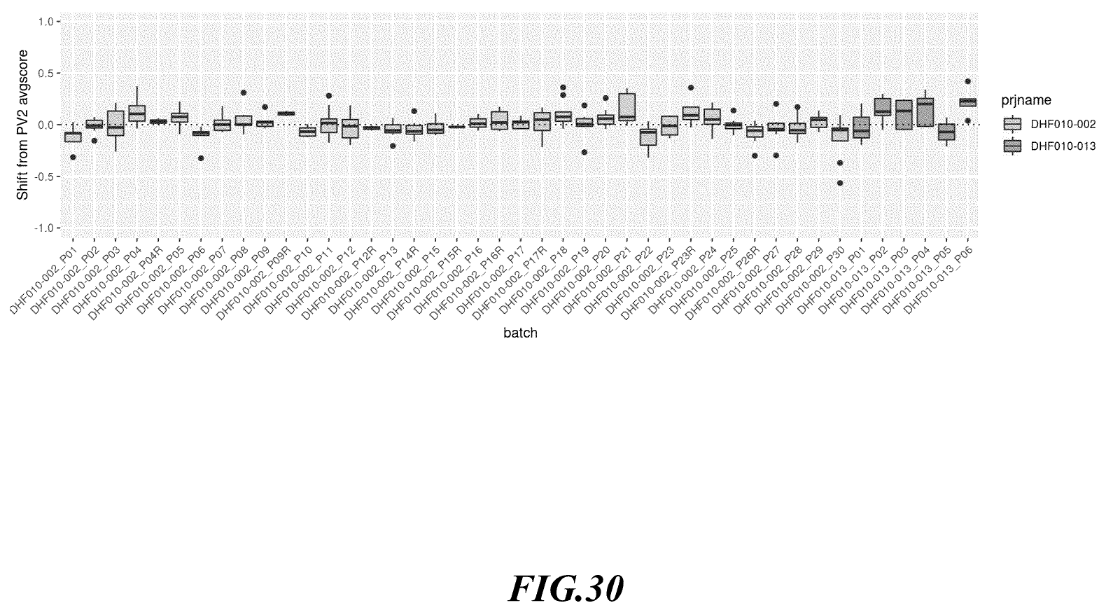

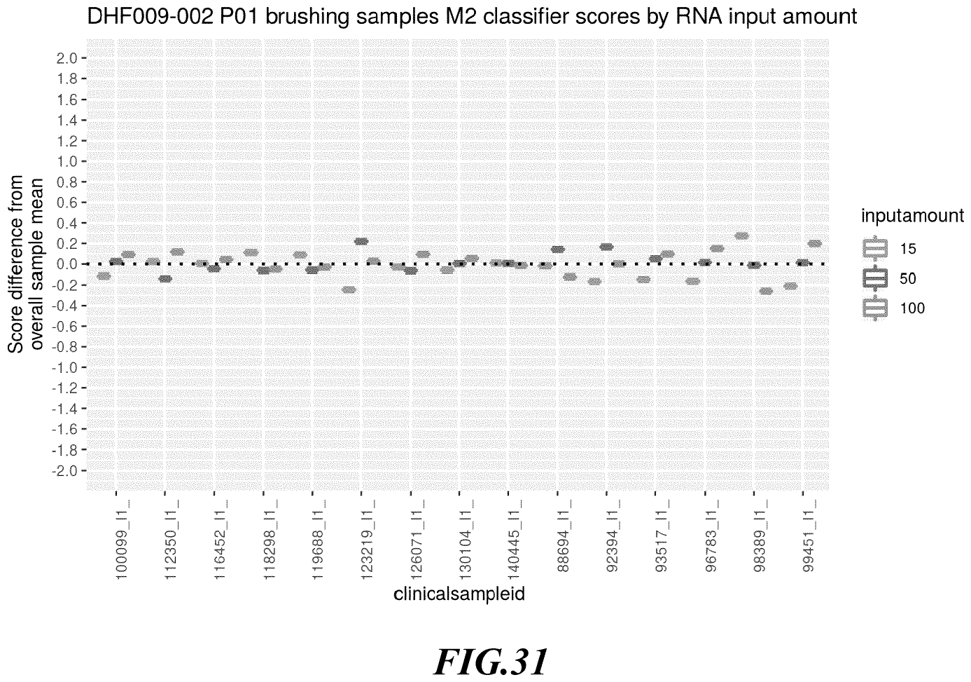

Analytical verification studies were performed on a locked assay system in order to fully characterize the system performance relative to pre-defined specifications prior to unblinding the clinical validation test set. The verification studies include reagent verification (vendor quality assessment, multiple lot qualification of assay components and control material, reagent stability, reagent freeze-thaw stability, etc.) as well as analytical verification (pre-analytical factors such as brush storage and shipping, reproducibility (intra-run, inter-run, and inter-lab), analytical sensitivity by total RNA input titration, and analytical specificity such as blood or genomic DNA). As can be seen in FIG. 30, the same five patient samples were run in 37 development and 6 verification plates/batches. A total standard deviation of ˜4% of the score range across all batches was observed, meeting the analytical product requirements. FIG. 31 shows a graph of fifteen different patient sample RNAs tested at 15, 50, or 100 ng total RNA input and the associated score difference from the overall sample mean. A score standard deviation of ˜4% of score range treating 15 ng, 50 ng, and 100 ng of RNA as replicates equivalent to replicates of 50 ng, meeting test requirements.

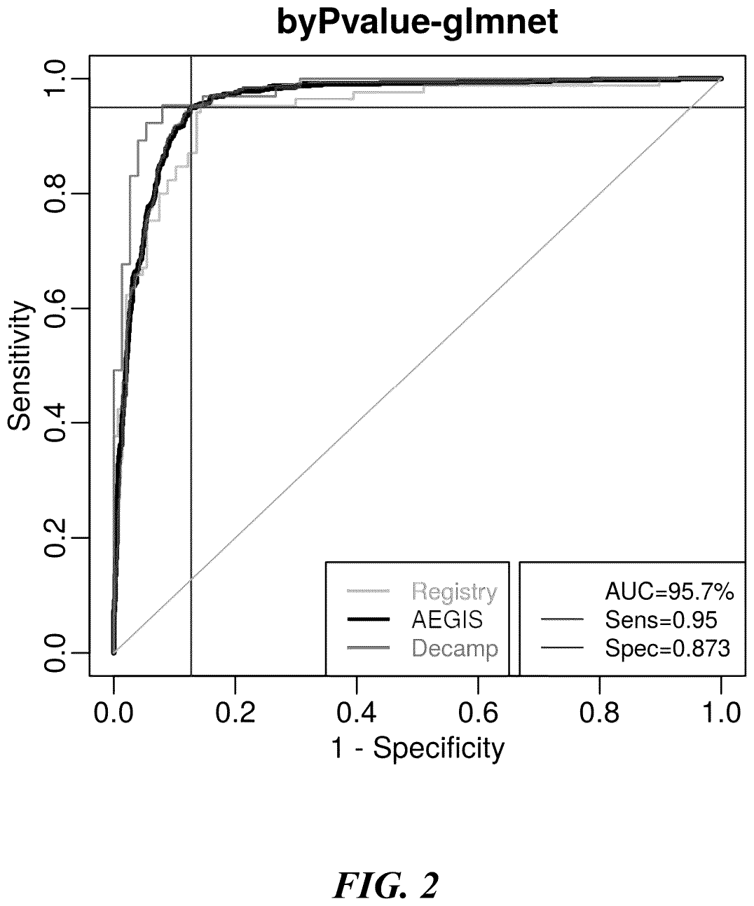

As can be seen in Table 13 and FIG. 2, using the algorithm in conjunction with the expression data from as few as 5 genes to as many as 10,000 genes generated a smoking status score which can differentiate current smokers from former smokers with an AUC >95% (FIG. 1) and a sensitivity of >0.95 and specificity of >0.85 as can be seen in FIG. 2.

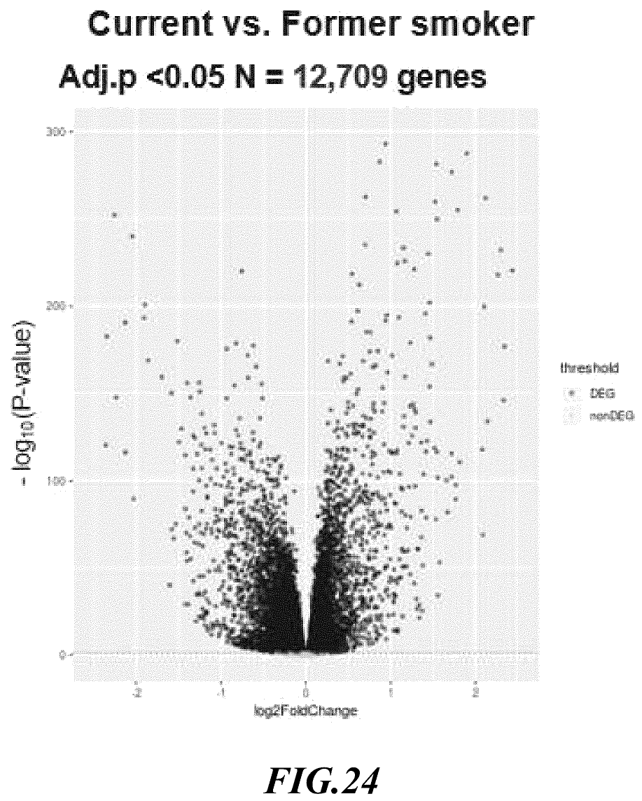

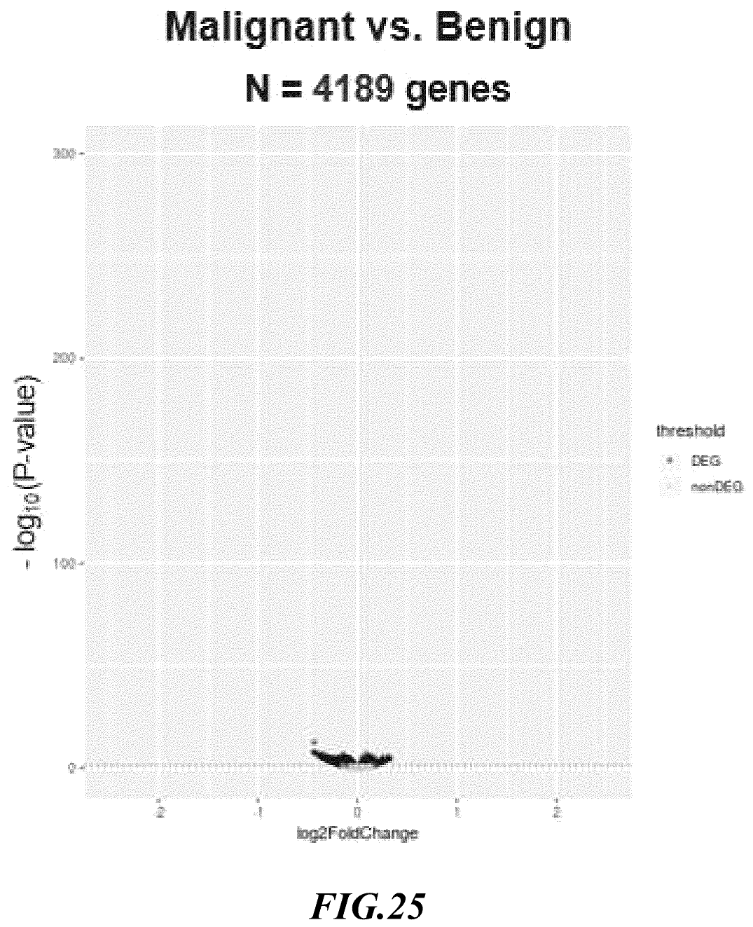

As can be seen in FIG. 24 and FIG. 25, the genomic signal obtained between current versus former smokers (12,709 genes) is a much stronger signal than the genomic signal obtained between samples obtained from subjects diagnosed with malignant versus benign tumors (4,189 genes).

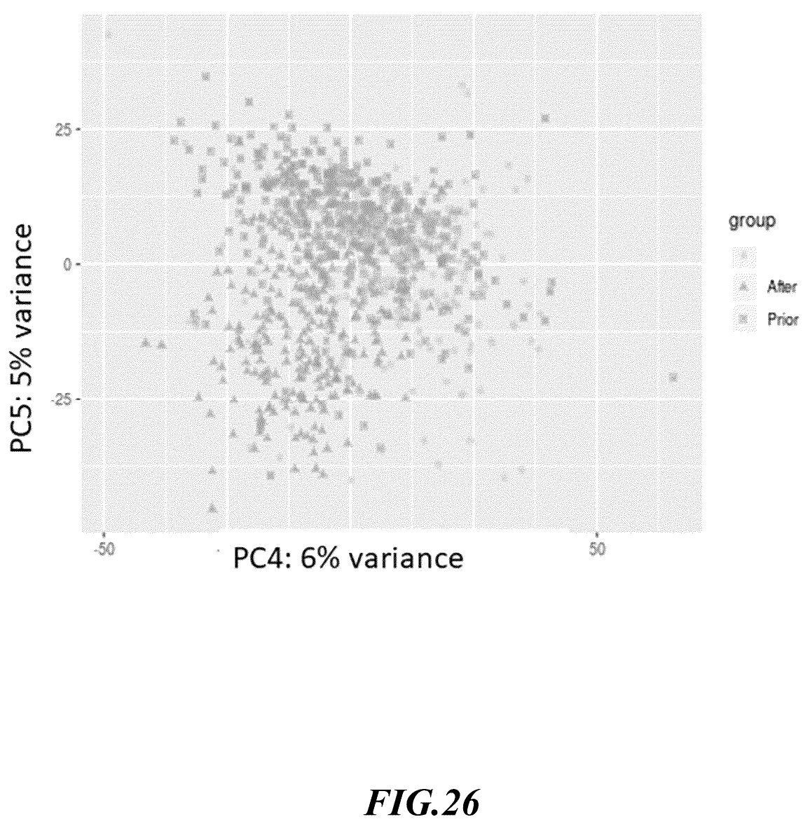

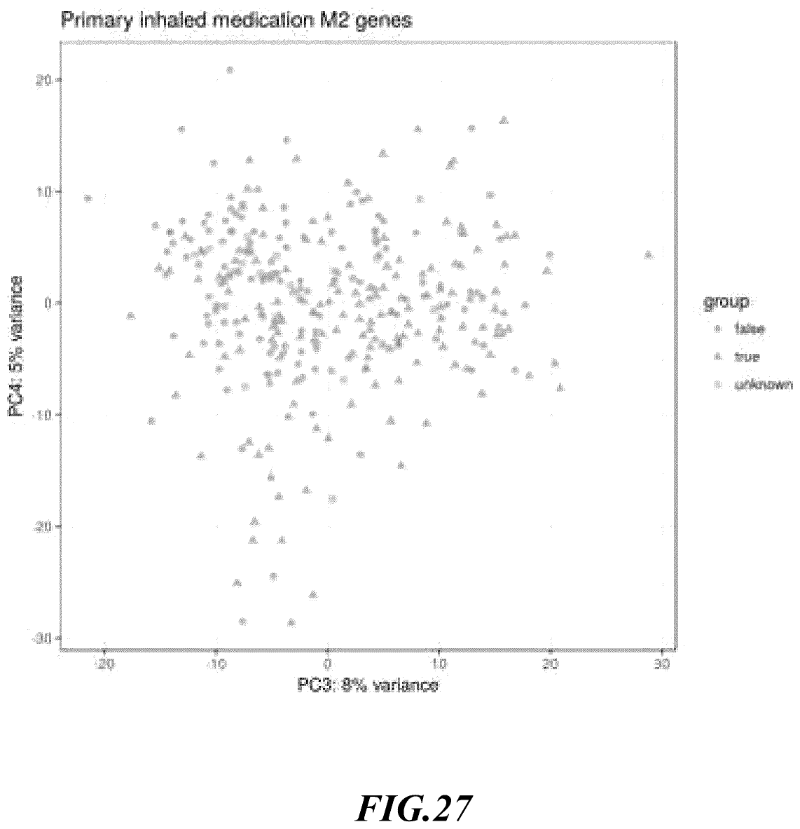

In order to improve the signal between benign and malignant samples, the timing of specimen collection was analyzed. FIG. 26 shows a graph that shows the genomic variance between samples from the same subjects, depending on the timing of collection. It was also noticed that the use of inhaled medication impacts gene expression, as can be seen in FIG. 27 which shows a graph of the variance differences between samples taken from subjects who had and subjects who had not been exposed to oral medications prior to sample collection.

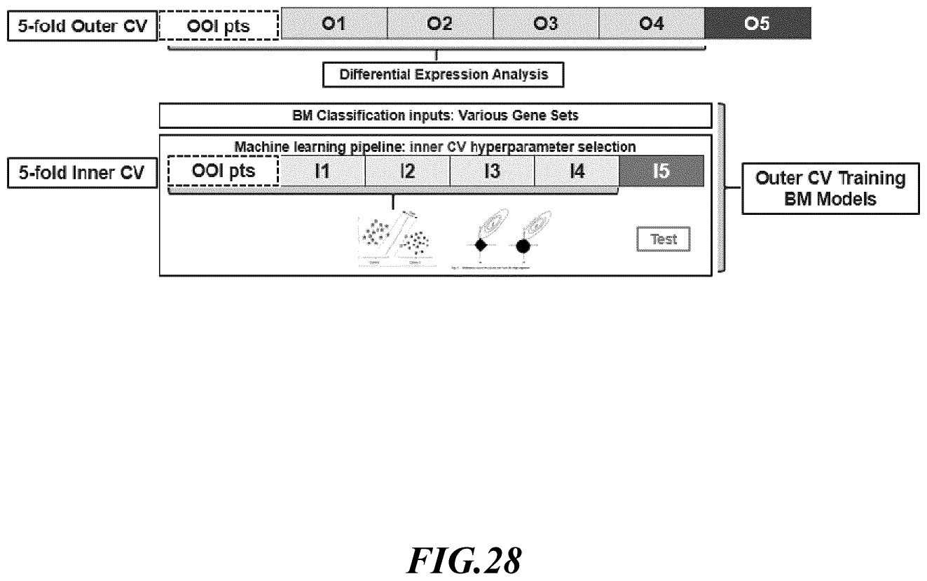

In order to improve performance of the classifier with the additional parameters, a nested cross validation (CV) and model selection protocol was implemented. The protocol includes performing at least 10 repeats of the cross validations to measure performance variability, wherein each cross validation analyzes the differential expression associated with a different parameter. A first feature selection method is utilized in which differentially expressed genes, unsupervised clusters of genes, and interaction terms of clinical variables and selected genes are analyzed. Second, a machine learning algorithm is then applied to identify the inner cross validation hyperparameter selection, as can be seen in FIG. 28. The machine learning method applies support vector machine models (SVM), penalized regression models (i.e., LASSO, Ridge regression), and tree-based methods (i.e. random forest, Xgboost). This pipeline is applied to build and test hundreds of models using many combinations of the methods.

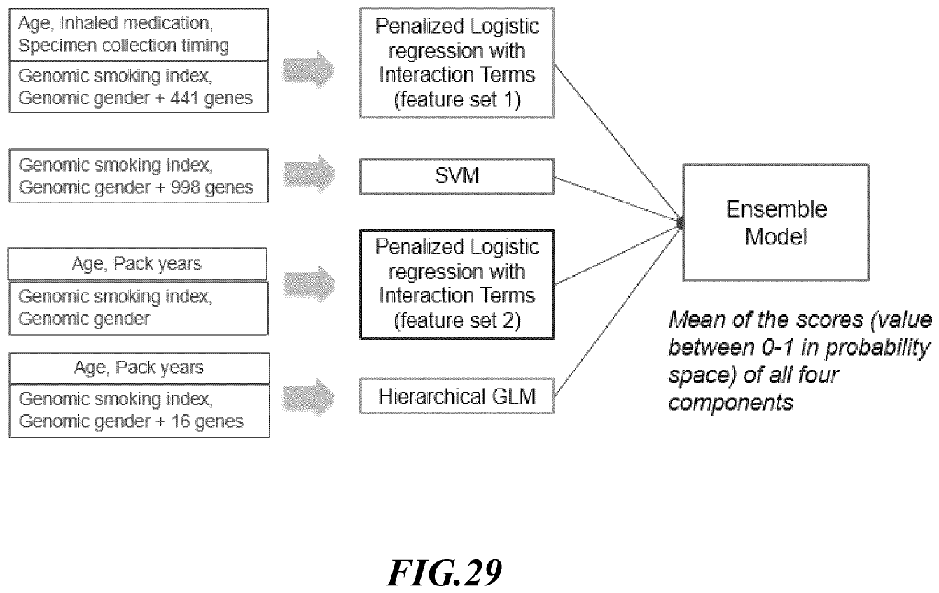

Using the above protocol, the six models were chosen to score the validation sample set. FIG. 29 shows an example of a protocol in which a penalized logistic regression with interaction terms (feature set 1), an SVM, a penalized logistic regression with interaction terms (feature set 2) and a hierarchical GLM were applied to produce an ensemble model used to score the validation sample set. Feature set 1 included the clinical features of age, inhaled medication and specimen timing in conjunction with the genomic features of the genomic smoking index genes, genomic gender, and 441 additional genes. Feature set 2 included the clinical features of age and pack year in conjunction with the genomic features of the genomic smoking index and genomic gender.

### Example 2: Validation of an Algorithm to Determine Smoking Status by Gene Expression from Lung Bronchial Epithelial Cells

The algorithm of Example 1 was applied to an independent test set comprising bronchial epithelial tissue gathered from subjects with either benign (B) or malignant (M) tumors. The subjects were either former smokers or current smokers.

Table 13 indicates the number of samples and the descriptions of the samples from the cohorts used: Aegis I/II and the Percepta Registry.

TABLE 13
Cohort samples used in validation

| Cohort | Description | Number |
|---|---|---|
| Aegis I/II | Within indication | 246 |
| Percepta Registry | Within indication */ | 121*/45** |
| | Local Benign** | |
| Total | 142 | 367/412 |

Patients with adjudicated benign or malignant labels were used to calculated sensitivity and specificity for * samples. Local benign patients (**), without adjudicated labels, were added for computing ROM, NPV (negative predictive value) and PPV (positive predictive value).

Table 14 outlines the patient demographics of the samples used from each cohort.

TABLE 14
Clinical variables of cohort samples used in validation

| | | | | Percepta Registry | |
|---|---|---|---|---|---|
| | | AEGIS | AEGIS | CVP-Within | CVP- |
| | | I | II | Indication | Local B |
| Characteristic | | (N = 109) | (N = 137) | (N = 121) | (N = 45) |
| Sex | Female | 41 | 42 | 58 | 26 |
| | Male | 68 | 95 | 63 | 19 |
| Median age (IQR) | | 62 (54-70) | 63 (55-71) | 65 (58-71) | 65 (56-71) |
| Race | White | 84 | 108 | 93 | 39 |
| | Black | 16 | 26 | 25 | 4 |
| | Other | 9 | 3 | 3 | 1 |
| | Unknown | 0 | 0 | 0 | 1 |
| Smoking | Current | 47 | 60 | 47 | 26 |
| status | Former | 62 | 77 | 74 | 19 |

| | | | | |
|---|---|---|---|---|
| Cumulative | 36 (23-60) | 32 (19-53) | 35 (20-60) | 31 (20-46) |
| tobacco use | | | | |
| Median Pack | | | | |
| Year (IQR) | | | | |

Table 15 outlines additional clinical variables of the cohort samples used in validation.

TABLE 15
Validation samples and associated clinical variables

| | | | | Percepta Registry | |
|---|---|---|---|---|---|
| | | | | CVP-Within | |
| | | AEGIS I | AEGIS II | Indication | CVP-Local B |
| Characteristic | | (N = 109) | (N = 137) | (N = 121) | (N = 45) |
| Lesion size | Infiltrate | 7 | 5 | 0 | 0 |
| | <2 cm | 28 | 57 | 57 | 23 |
| | 2 to 3 cm | 23 | 25 | 24 | 5 |
| | >3 cm | 37 | 37 | 31 | 13 |
| | Unknown | 14 | 13 | 9 | 4 |
| Lesion location | Central | 31 | 41 | 7 | 3 |
| | Peripheral | 40 | 68 | 106 | 38 |
| | Central and peripheral | 28 | 25 | 0 | 0 |
| | Unknown | 10 | 3 | 8 | 4 |
| Lung-cancer | Small-cell | 4 | 4 | 1 | — |
| histologic type | Non-small-cell | 43 | 57 | 43 | — |
| | Adeno | 21 | 37 | 25 | — |
| | Squamous | 13 | 13 | 10 | — |
| | Large-cell | 2 | 2 | 0 | — |
| | Not specified | 7 | 5 | 8 | — |
| | Other | 0 | 0 | 2 | — |
| | Unknown | 1 | 2 | 6 | — |
| Diagnosis of a | Fibrosis | 0 | 1 | 0 | — |
| benign condition | Granuloma | 10 | 16 | 10 | — |
| | Infection | 20 | 16 | 15 | — |
| | Inflammation | 0 | 1 | 2 | — |
| | Multiple | 5 | 3 | 0 | — |
| | Other | 11 | 14 | 2 | — |
| | Unknown | 15 | 23 | 40 | — |

Table 16 shows a breakdown of the clinical validation dataset broken down by pre-test risk of malignancy. Nineteen percent (80 samples) had a low risk, 35% (144 samples) had a high risk, and 46% (188 samples) had an intermediate risk.

TABLE 16
Pre-test risk of malignancy within validation samples

| | | Low risk | | Intermediate risk | | High risk | | |
|---|---|---|---|---|---|---|---|---|
| Cohort | Description | Benign | malignant | benign | malignant | benign | malignant | Total |
| AEGIS I &II | Within indication | 56 | 2 | 58 | 24 | 21 | 85 | 246 |
| Registry | Within | 12 | 2 | 44 | 29 | 13 | 21 | 121* |
| | indication* | | | | | | | |
| | Local Benign** | 8 | . | 33 | . | 4 | . | 45** |

| | |
|---|---|
| Total | 367*/412** |

The final validation set was composed of 246 samples from the Aegis cohort after excluding samples with insufficient remaining RNA and excluding those samples that failed the sequencing QC metrics. To calculate the Risk of malignancy in each risk category of the validation dataset, the number of samples from subjects diagnosed with a malignant tumor in a risk category was divided the total number of samples in the category. The results are summarized in Table 17 below.

TABLE 17
Risk of malignancy (ROM) within the validation dataset.

| | | Low risk | | Intermediate risk | | High risk | | |
|---|---|---|---|---|---|---|---|---|
| Cohort | | Benign | malignant | benign | malignant | benign | malignant | Total |
| AEGIS | | 56 | 2 | 58 | 24 | 21 | 85 | 246 |
| Registry | Adjudicated labels | 12 | 2 | 44 | 29 | 13 | 21 | 121 |
| | Local Benign | 8 | . | 33 | . | 4 | . | 45 |

| | | | |
|---|---|---|---|
| ROM* | 4/80 = 5% | 53/188 = 28.2% | 106/144 = 73.6% |

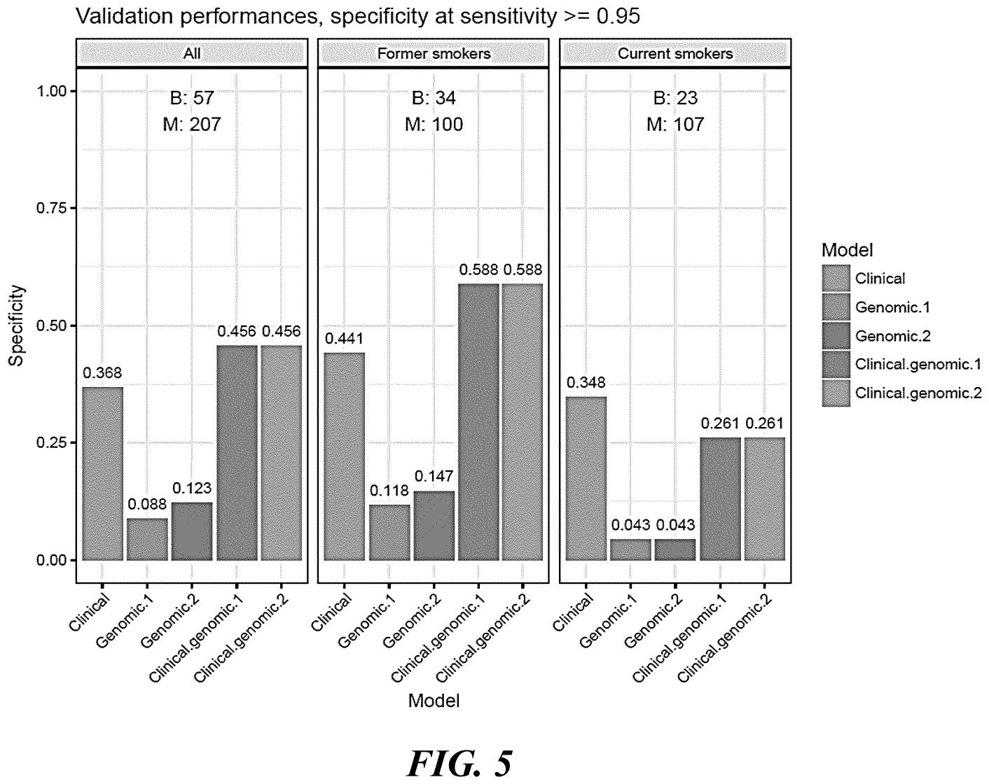

The specificity of the algorithm as applied to the samples was measured with a sensitivity set at great than 95% for all samples. As can be seen in FIG. 5, the specificity for the overall test set was 45.6%. The specificity for samples from former smokers only was 58.8%. The specificity for samples from current smokers only was 26.1%. Table 26 below summarizes the results.

TABLE 26
Validation performances, specificity at
sensitivity greater than or equal to 0.95

| | | | | Clinical | Clinical |
|---|---|---|---|---|---|
| | | Geno- | Geno- | Geno- | Geno- |
| Samples | Clinical | mic 1 | mic 2 | mic 1 | mic 2 |
| All (57 benign, | 0.368 | 0.088 | 0.123 | 0.456 | 0.456 |
| 207 malignant) | | | | | |
| Former Smokers | 0.441 | 0.118 | 0.147 | 0.588 | 0.588 |
| (34 benign, | | | | | |
| 100 malignant) | | | | | |
| Current Smokers | 0.348 | 0.043 | 0.043 | 0.261 | 0.261 |
| (23 benign, | | | | | |
| 107 malignant) | | | | | |

The final performance of classifier on the validation dataset is summarized in Table 18.

TABLE 18
Final performance of the classifier on the Validation Dataset

| | | | % | | |
|---|---|---|---|---|---|
| Product Features | ROM | NPV/PPV | impact | Sensitivity | Specificity |
| Down-classify | 5% | 100% NPV | 53.1% | 100% | 55.9% |
| Low to Very Low | | [90.7-100] | | [39.8-100] | [43.3-67.9] |
| Down-classify | 28.2% | 91.0% NPV | 29.4% | 90.6% | 37.3% |
| Intermediate to Low | | [80.8-96.0] | | [79.3-96.9] | [27.9-47.4] |
| Up-classify | 28.2% | 65.4% PPV | 12.2% | 28.3% | 94.1% |
| Intermediate to High | | [43.8-82.1] | | [16.8-42.3] | [87.6-97.8] |
| Up-classify | 73.6% | 91.5% PPV | 27.3% | 34.0% | 91.2% |
| High to Very High | | [77.9-97.0] | | [25.0-43.8] | [76.3-98.1] |

During the adjudication process for Registry samples, some patient samples did not yield adjudicated benign versus malignant samples. These are all local benign samples when they went into the adjudication. This subgroup is referred to as “local benign.” Local benign patients were excluded when calculating sensitivity and specificity. In other words, sensitivity and specificity were calculated based on adjudicated labels. NPV, PPV, and % impact are all functions of the risk of malignancy (ROM) (estimated including local benign patients), sensitivity, and specificity (both estimated excluding local benign patients).

In the training set, clinical-genomic classifiers slightly outperformed clinical-only classifiers, with higher improvement among former smokers. In the validation set, the overall performance of clinical-genomic classifiers is similar to clinical-only classifiers. In the validation set, clinical-genomic classifiers have a higher specificity (at greater than or equal to 95% sensitivity) than clinical-only classifier among former smokers. The performance of both the clinical-only classifiers and the clinical-genomic classifiers varied across the different subsets of samples.

The classifier was shown to perform four types of risk reclassification, as can be seen in FIG. 32. The application of the classifier to the validation training set is summarized in Table 19.

TABLE 19
Application of classifier to down-classify
and up-classify cancer risk

| | | % | | |
|---|---|---|---|---|
| Product Features | NPV/PPV | impact | Sensitivity | Specificity |
| Down-classify | 100% NPV | 53.1% | 100% | 55.9% |
| Low to Very Low | [90.7-100] | | [39.8-100] | [43.3-67.9] |
| Down-classify | 91.0% NPV | 29.4% | 90.8% | 37.3% |
| Intermediate to Low | [80.8-96.0] | | [79.3-96.9] | [27.9-47.4] |
| Up-classify | 65.4% PPV | 12.2% | 28.3% | 94.1% |
| Intermediate to High | [43.8-82.1] | | [16.8-42.3] | [87.6-97.8] |
| Up-classify | 91.5% PPV | 27.3% | 34.0% | 91.2% |
| High to Very High | [77.9-97.0] | | [25.0-43.8] | [76.3-98.1] |

The classifier was trained on samples from four cohorts: Aegis I/II, Percepta Registry and DECAMP and prospectively validated on three independent cohorts: Aegis I/II and Percepta Registry. The models used in the classifier incorporated interaction terms that stabilized the independent signals in the genomic data arising from smoking status (current v. former), collection time (prior v. after) and the use of inhaled medication (yes/no). The classifier was shown to maintain the core-feature for down-classifying intermediate risk patients to low-risk with a 90% negative predictive value (NPV). The classifier down-classified low risk patients to very low risk patients with a PPV of greater than 99%. The classifier up-classified intermediate risk patients to high risk with a PPV of greater than 65%. The classifier up-classified high risk patients to very high with a PPV of greater than 90%.

### Example 3: Development of an Algorithm to Determine Smoking Status by Gene Expression from Nasal Epithelial Cells

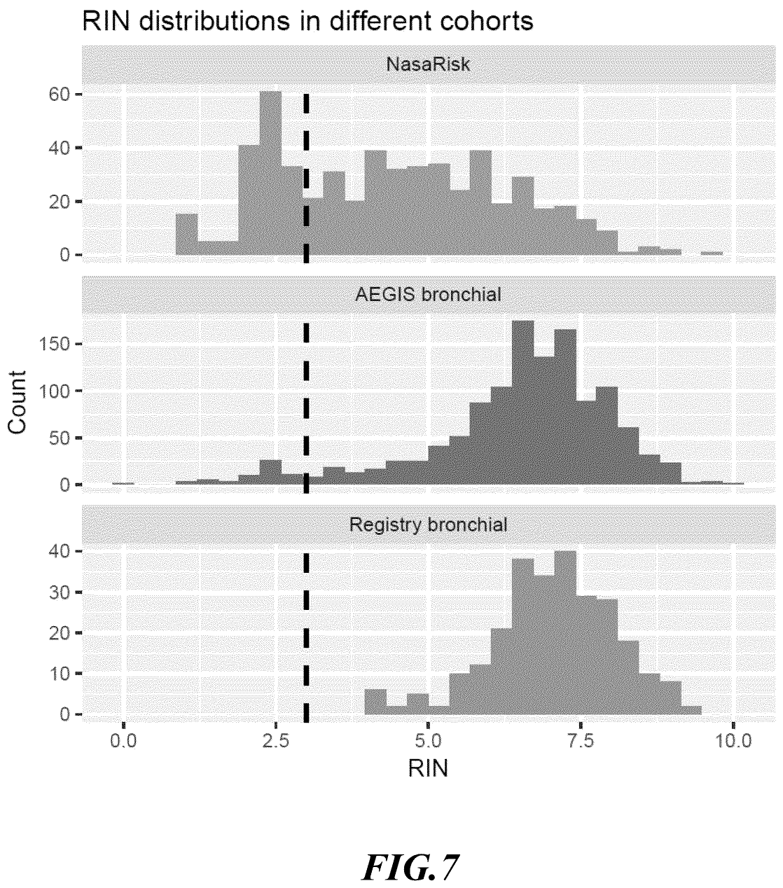

The algorithm was then applied to nasal brushing samples to classify benign versus malignant (B v M) classes of subjects. DNA sequencing (Unified Assay) data was generated from AEGIS nasal brushing samples. Unlike bronchial samples, NasaRisk (AEGIS nasal samples) have a significantly lower RNA integrity number (RIN) than AEGIS bronchial samples and Percepta registry bronchial samples, as can be seen in FIG. 7. Samples with low RINs may have a lower quality RNAseq gene expression measurement.

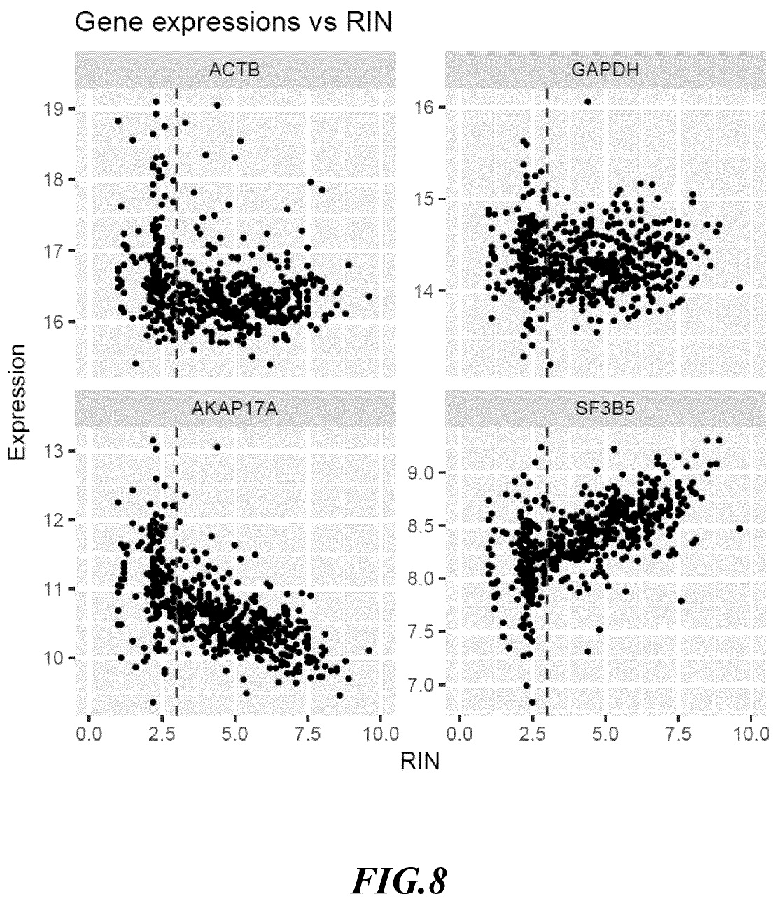

To test the variation of gene expression in nasal brushing samples, the gene expression of four genes, ACTB, GADPH, AKAP17A, and SF3B5 were measured in 545 NasaRisk primary training set samples. ACTB and GAPDH are two housekeeping genes. AKAP17A and SF3B5 are genes with expression levels that were found to be strongly correlated with RIN in the sample set. FIG. 8 shows a graph of RIN versus gene expression for each of the four genes in each of the 545 samples. Among the samples with RIN<3, the gene expression measurements had a larger variation.

Similar to the process of Example 1, next generation sequencing of RNA from 545 samples of nasal epithelial cells were analyzed using the same machine learning process of Example 1. The RNA sequencing data was normalized. A genomic classifier was then built based on the smoking status of the subjects (current v. former).

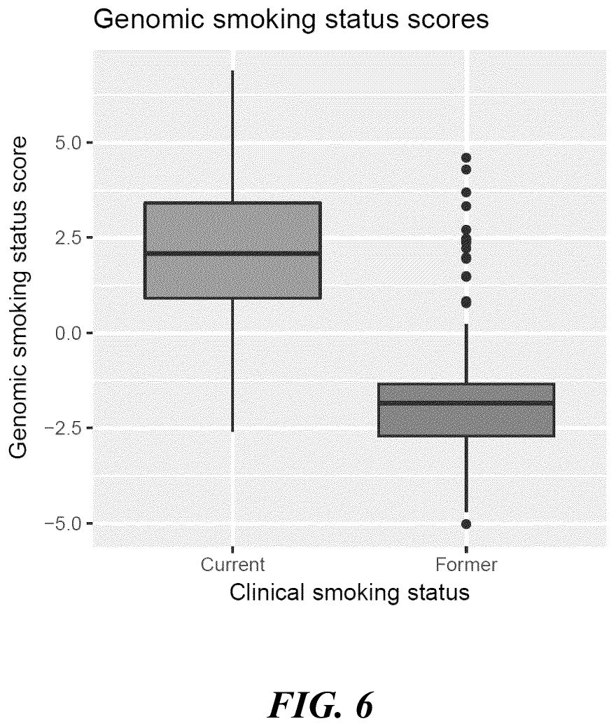

A genomic classifier for smoking stats was built to show that smoking status could be accurately predicted using gene expression and to use the genomic smoking status predictions as a predictor in benign versus malignant classifications. The genomic classifier was built using a Support Vector Machine (SVM) model. Using 0 as the cutoff value, it achieved an accuracy rate of 0.905 (493/545). The genomic smoking status scores created using the model to identify smoking status can be seen in FIG. 6.

The data was then analyzed for differential gene expression between subjects with benign tumors (B) and malignant tumors (M).

The samples were divided into a primary training set, a prior cancer training set, and an OOI training set, as can be seen in Table 4 below. Training set assignments were partially random. All bronchoscopy indeterminate samples were assigned using the methods described herein. Primary group samples were bronchoscopy positive or indeterminate with no prior cancer, could be current or former smokers, and had not been diagnosed with metastatic cancer to the lung. Prior cancer group samples were from subjects previously diagnosed with cancer, could be from current or former smokers, and had not been diagnosed with metastatic cancer to the lung. OOI group samples were from never smoker subjects or from subjects diagnosed with metastatic cancer to the lung

TABLE 4
Number of training set samples:

| | Cancer Diagnosis: | Cancer Diagnosis: | |
|---|---|---|---|
| Training Set Group | Benign | Malignant | Total |
| Primary | 88 | 457 | 545 |
| Prior Cancer | 3 | 158 | 161 |
| OOI | 0 | 178 | 178 |
| Total | 91 | 793 | 884 |

As described above, the samples in the primary training set included samples from subjects classified as current and former smokers and well as a varying pre-test risk of malignancy (ROM), calculated as described in Examples 1 and 2. The number of samples from current and former smokers as well as the pre-test ROM classification of the primary training set can be seen in Tables 5 and 6 below.

TABLE 5
Number of training set samples:

| | Cancer Diagnosis: | Cancer Diagnosis: | |
|---|---|---|---|
| Smoking Status | Benign | Malignant | Total |
| Current Smokers | 27 | 235 | 262 |
| Former Smokers | 61 | 222 | 283 |
| Total | 88 | 457 | 545 |

TABLE 6
Number of training set samples:

| | Cancer Diagnosis: | Cancer Diagnosis: | |
|---|---|---|---|
| Pre-Test ROM | Benign | Malignant | Total |
| High | 16 | 366 | 382 |
| Intermediate | 31 | 30 | 61 |
| Low | 22 | 1 | 23 |
| Unknown | 19 | 60 | 79 |
| Total | 88 | 457 | 545 |

Analysis of samples with a RIN greater than or equal to 3

To improve the performance of the classifier, samples with a RIN<3 were removed, leaving 385 of the 545 samples. The number of samples from current and former smokers as well as the pre-test ROM classification of the primary training set can be seen in Tables 7 and 8 below.

TABLE 7
Number of training set samples:

| | Cancer Diagnosis: | Cancer Diagnosis: | |
|---|---|---|---|
| Smoking Status | Benign | Malignant | Total |
| Current Smokers | 16 | 159 | 175 |
| Former Smokers | 39 | 171 | 210 |
| Total | 55 | 330 | 385 |

TABLE 8
Number of training set samples:

| | Cancer Diagnosis: | Cancer Diagnosis: | |
|---|---|---|---|
| Pre-Test ROM | Benign | Malignant | Total |
| High | 14 | 272 | 286 |
| Intermediate | 18 | 22 | 40 |
| Low | 11 | 1 | 12 |
| Unknown | 12 | 35 | 47 |
| Total | 55 | 330 | 385 |

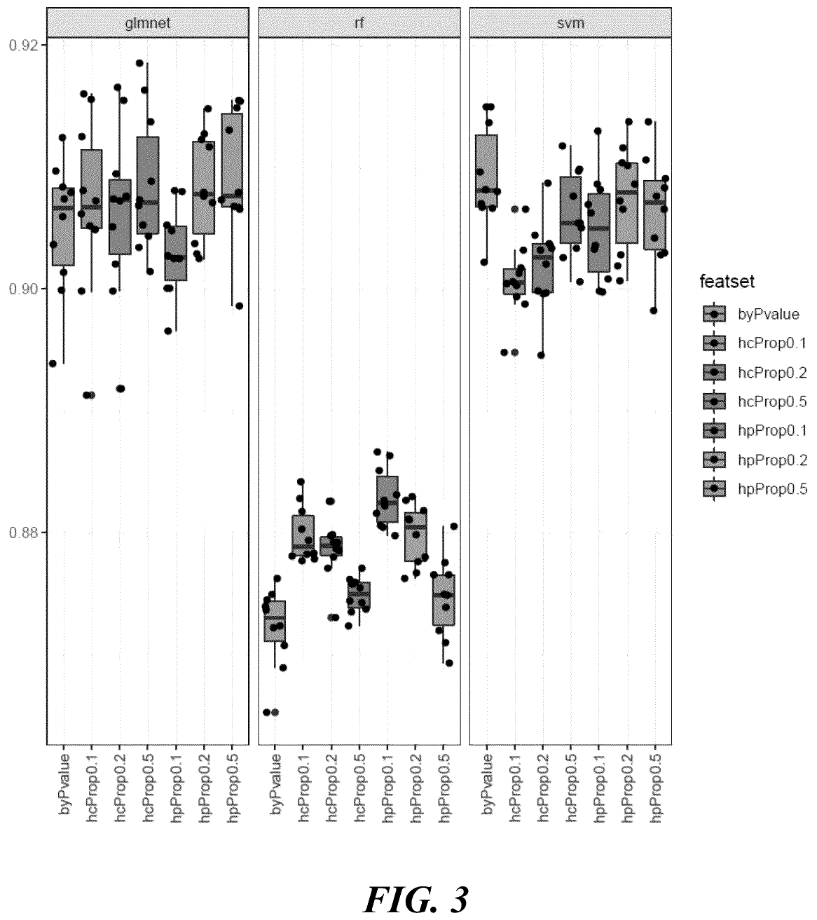

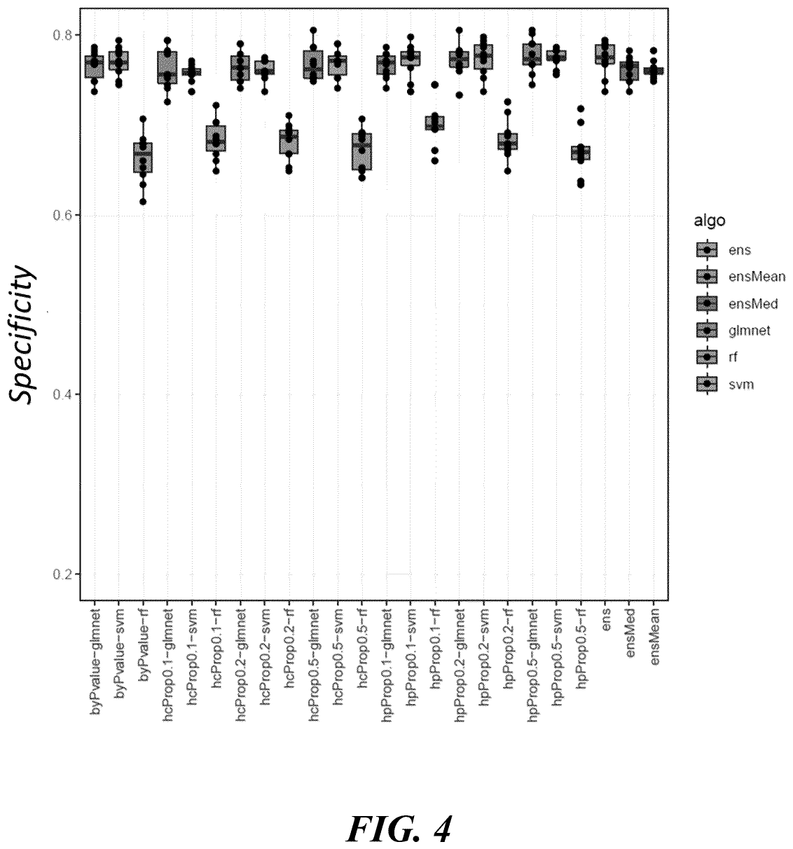

A set of models was identified, each containing 100 genes or more, to identify current smokers from former smokers with an AUC of >90% as can be seen in FIG. 3. A sensitivity of 0.90 and a specificity of 0.78 was obtained as can be seen in FIG. 4. The genes used were also present in the bronchial derived model of Example 1.

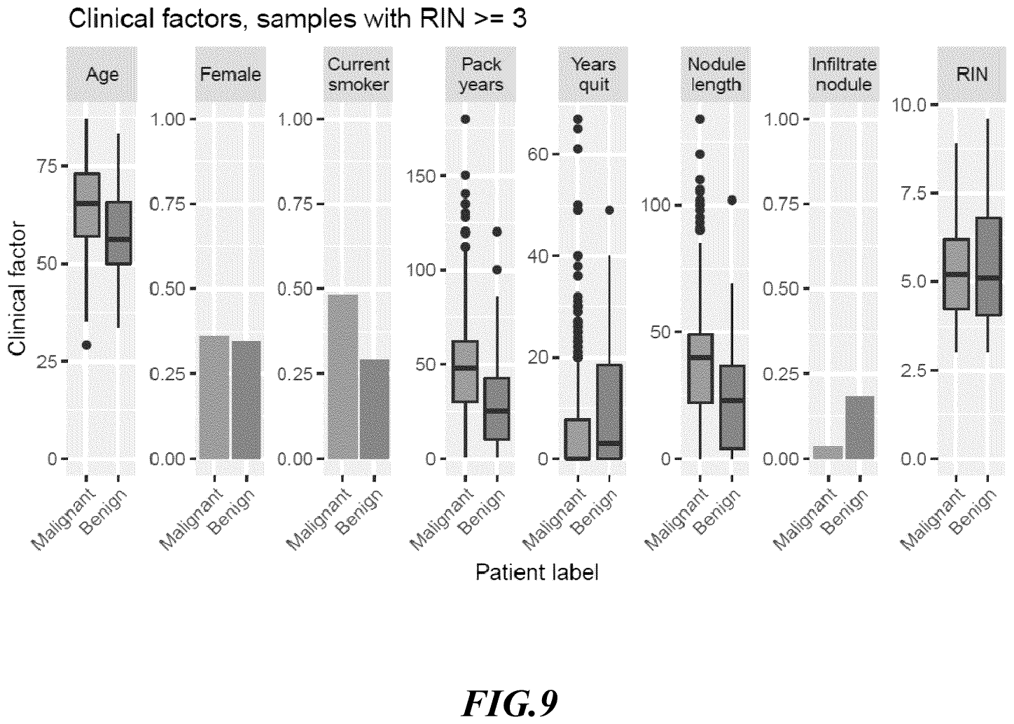

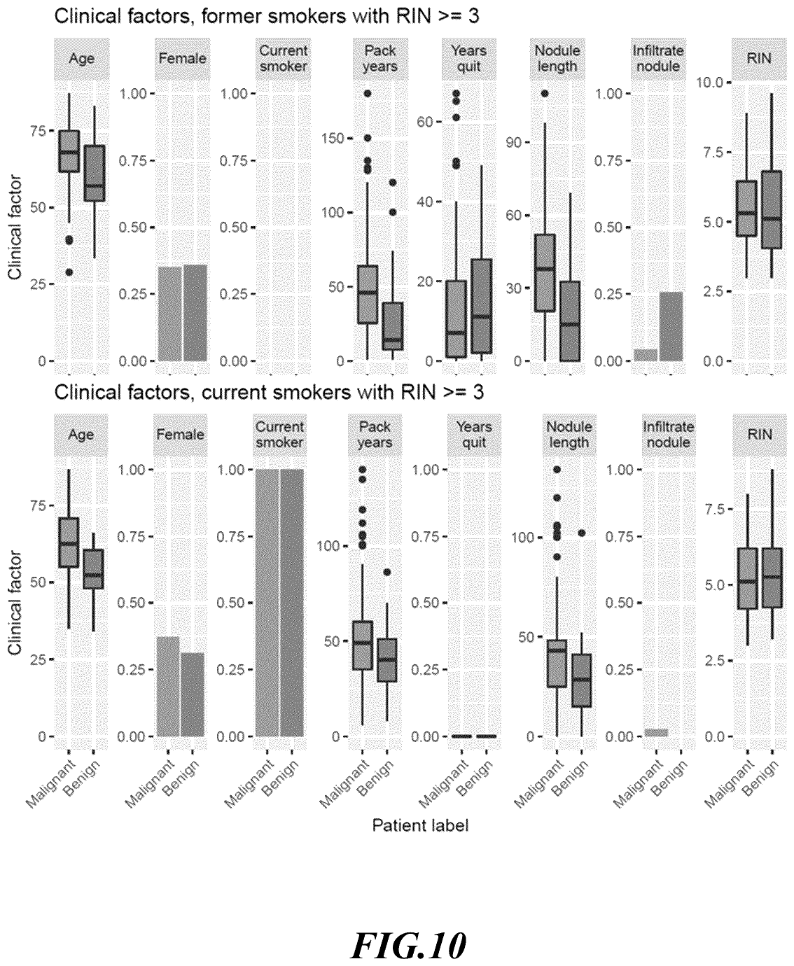

FIG. 9 shows the variation in clinical factors throughout the samples between samples obtained from subjects with benign or malignant tumors. The clinical factors include age, gender, smoking status, pack years, years since smoking, nodule length, infiltrate nodule, and RIN. Age, pack-year and nodule length have apparent differences between benign and malignant samples. In current smokers, there are more malignant samples than benign samples. Furthermore, when clinical factors were additionally analyzed separately for current and former smokers, as can be seen in FIG. 10, pack year and nodule length showed a greater difference between benign and malignant samples in former smokers than in current smokers. Additionally, years since quitting smoking showed a greater difference between benign and malignant samples in former smokers than current smokers.

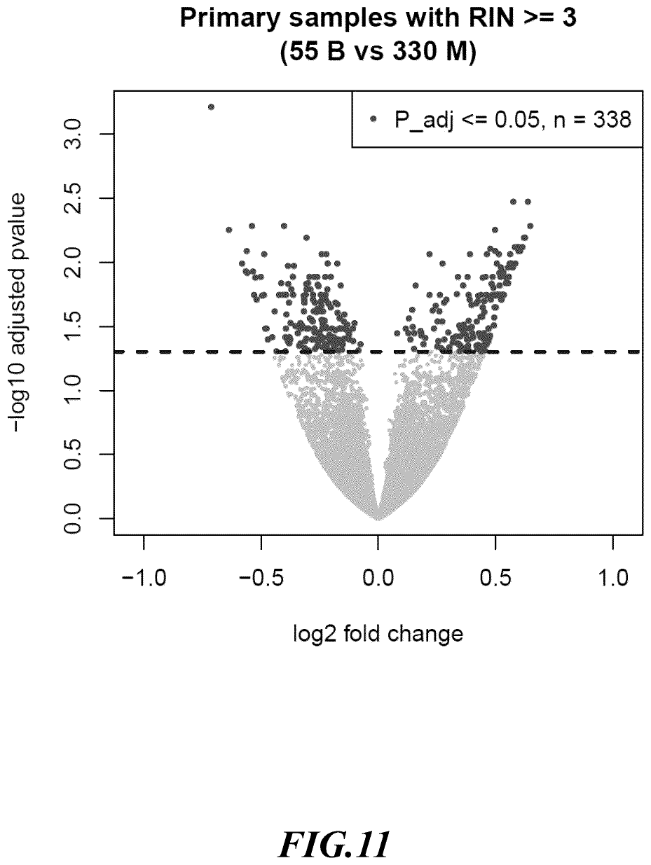

Seeing that the clinical factors helped to differentiate benign versus malignant samples, a negative-binomial test in a DESeq2 package that included smoking status (current/former) and gender (male/female) as covariates was applied to the data set. As can be seen in FIG. 11, a modest number of genes have a significant difference between samples from subjects with benign tumors versus subjects with malignant tumors. Based on adjusted p-values, 338 genes were significantly different between B and M samples. No genes had a fold change greater than 2 and few genes had a fold change more than 1.5.

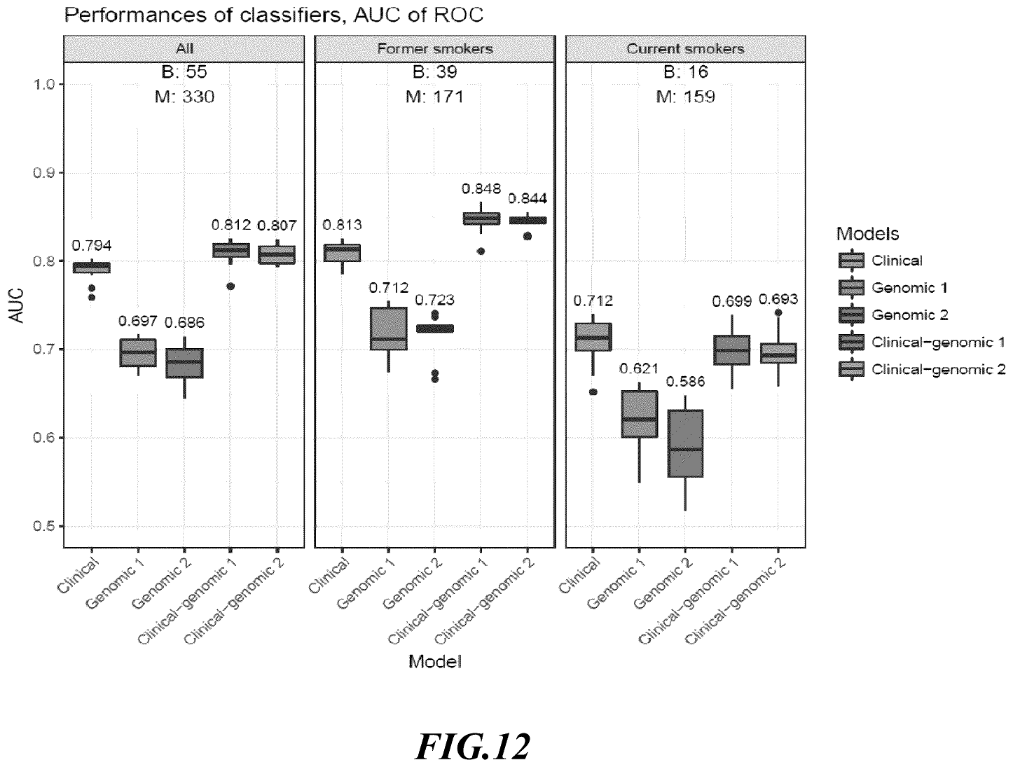

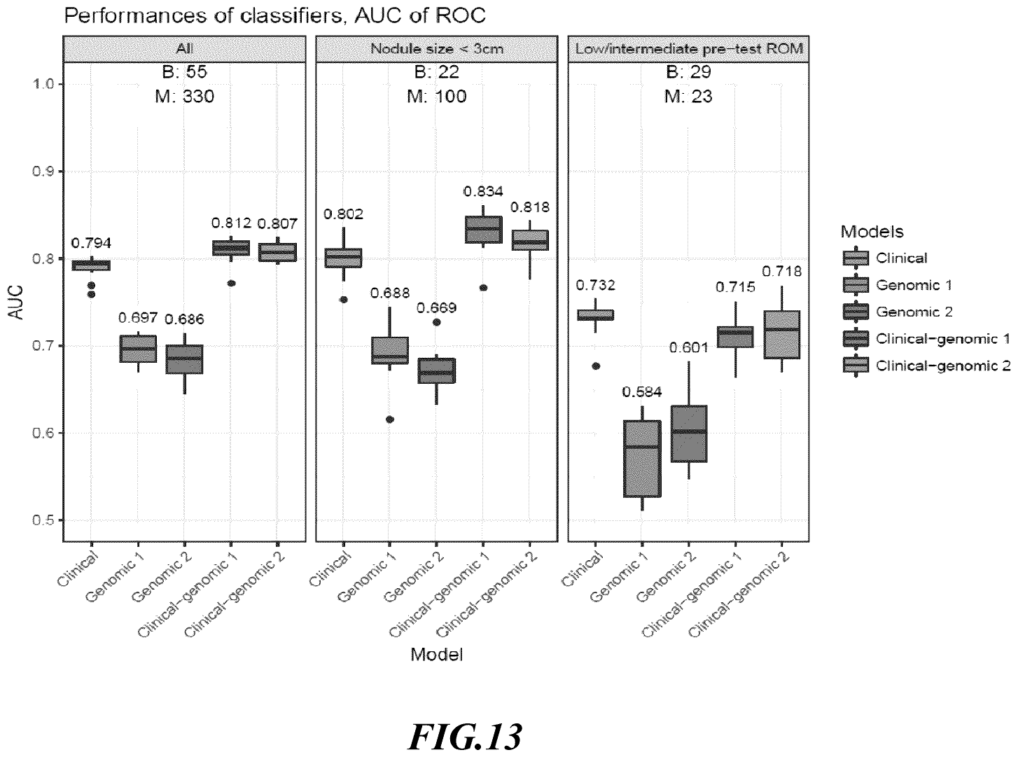

The performance of the classifiers were then tested, as can be seen in FIG. 12 and FIG. 13. Table 27 and Table 28 below summarize the results. All classifiers were evaluated by 5-fold cross-validation (CV) with 10 replicates. The AUC of ROC was used as the criterion for comparison. Performances were evaluated in all samples, former smokers only, and current smokers only. The top classifers from each category are shown. In all samples, clinical-genomic classifiers slightly outperform clinical only classifers. Genomic classifiers perform significantly worse than the other two types of classifiers. In samples with small nodules, clinical-genomic classifiers slightly outperform clinical only classifiers. In samples with low and intermediate pre-test ROMs, clinical genomic classifiers slightly underperform clinical only classifiers.

TABLE 27
Performances of classifiers, AUC of ROC

| | | | | Clinical | Clinical |
|---|---|---|---|---|---|
| | Clini- | Genomic | Genomic | Genomic | Genomic |
| Samples | cal | 1 | 2 | 1 | 2 |
| All (55 | 0.794 | 0.697 | 0.686 | 0.812 | 0.807 |
| benign, 330 | | | | | |
| malignant) | | | | | |
| Former | 0.813 | 0.712 | 0.723 | 0.848 | 0.844 |
| Smokers (39 | | | | | |
| benign, 171 | | | | | |
| malignant) | | | | | |
| Current | 0.712 | 0.621 | 0.586 | 0.699 | 0.693 |
| Smokers (16 | | | | | |
| benign, 159 | | | | | |
| malignant) | | | | | |

TABLE 28
Performances of classifiers, AUC of ROC

| | | | | Clinical | Clinical |
|---|---|---|---|---|---|
| | Clini- | Genomic | Genomic | Genomic | Genomic |
| Samples | cal | 1 | 2 | 1 | 2 |
| All (55 benign, | 0.794 | 0.697 | 0.686 | 0.812 | 0.807 |
| 330 malignant) | | | | | |
| Nodule size <3 | 0.802 | 0.688 | 0.669 | 0.834 | 0.818 |
| cm (22 benign, | | | | | |
| 100 malignant) | | | | | |
| Low/Interme- | 0.732 | 0.584 | 0.601 | 0.715 | 0.718 |
| diate pre-test | | | | | |
| ROM (29 | | | | | |
| benign, 23 | | | | | |
| malignant) | | | | | |

The clinical classifiers comprise input clinical factors: age, gender, smoking status, pack-year, years-since-quit, nodule length, and infiltrate nodule. The clinical classifiers were run with the following models: SVM, penalized GLM, and penalized GLM with interaction term.

The genomic classifiers comprise input from expression of genes chosen with various feature selection options and were run with the following models: SVM and penalized GLM.

The clinical-genomic classifiers comprise input clinical factors (age, gender, pack-year, years-since-quit, nodule length, infiltrate nodule) as well as genomic smoking status, and PIN. The clinical-genomic classifiers were run with the following models: SVM, penalized GLM, and penalized GLM with interaction terms.

### Example 4: Validation of an Algorithm to Determine Smoking Status by Gene Expression from Nasal Epithelial Cells

To validate the algorithm, samples were divided into a primary validation set group and a prior cancer validation set group, as can be seen in Table 9 below.

TABLE 9
Number of validation set samples:

| | Cancer Diagnosis: | Cancer Diagnosis: | |
|---|---|---|---|
| Training Set Group | Benign | Malignant | Total |
| Primary | 138 | 291 | 429 |
| Prior Cancer | 1 | 91 | 92 |
| Total | 139 | 382 | 521 |

As previously discussed in Example 3, validation samples with a RIN<3 were removed from the validation sample set. The number of samples from current and former smokers as well as the pre-test ROM classification of the primary validation set can be seen in Tables 10 and 11 below.

TABLE 10
Number of validation set samples:

| | Cancer Diagnosis: | Cancer Diagnosis: | |
|---|---|---|---|
| Smoking Status | Benign | Malignant | Total |
| Current Smokers | 32 | 94 | 126 |
| Former Smokers | 55 | 109 | 164 |
| Total | 7 | 203 | 290 |

TABLE 11
Number of validation set samples:

| | Cancer Diagnosis: | Cancer Diagnosis: | |
|---|---|---|---|
| Pre-Test ROM | Benign | Malignant | Total |
| High | 13 | 163 | 176 |
| Intermediate | 35 | 24 | 59 |
| Low | 36 | 1 | 37 |
| Unknown | 3 | 15 | 18 |
| Total | 87 | 203 | 290 |

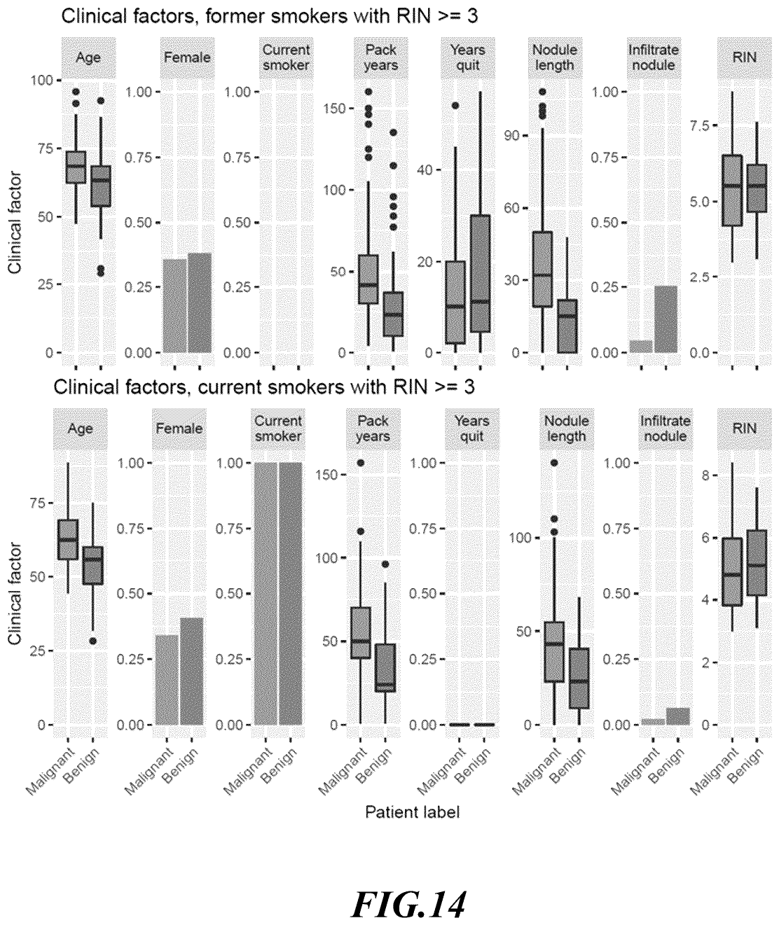

FIG. 14 shows the variation in clinical factors throughout the samples between samples obtained from subjects with benign or malignant tumors and between former smokers and current smokers. The clinical factors include age, gender, pack years, years since smoking, nodule length, infiltrate nodule, and RIN. Pack-year has apparent differences between benign and malignant samples that is greater than that seen in the training set.

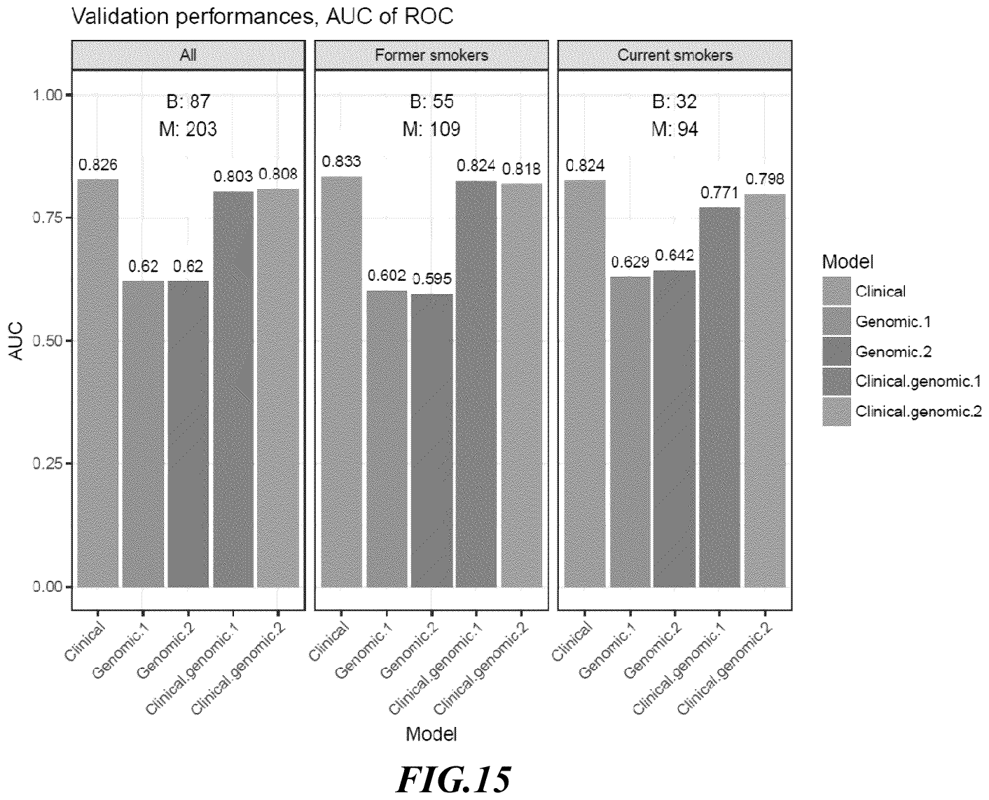

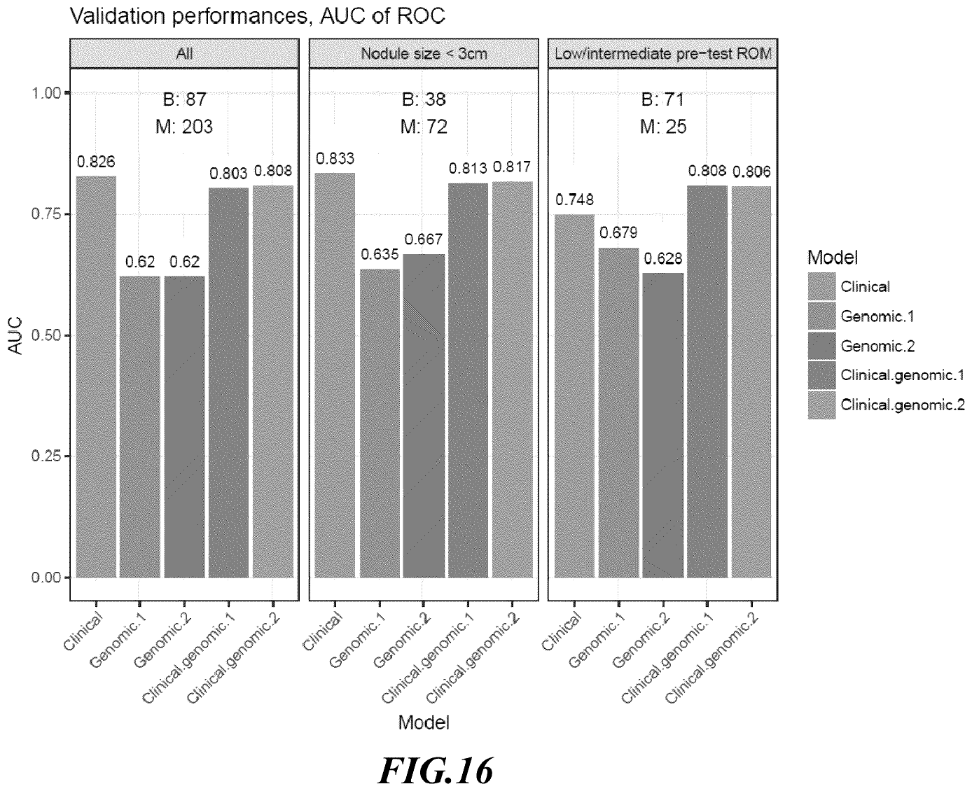

The validation performance of the classifiers were then tested, as can be seen in FIG. 15 and FIG. 16. Table 29 and Table 30 below summarize the results. All classifiers were evaluated by 5-fold cross-validation (CV) with 10 replicates. The AUC of ROC was used as the criterion for comparison. Performances were evaluated in all samples, former smokers only, and current smokers only. The top classifiers from each category are shown. Using AUC of ROC as a metric, clinical-genomic classifiers have slightly worse performance than clinical only classifiers in all three sample sets. Among current smokers, performance of clinical only and clinical-genomic classifiers are much better in validation set than in training set. Clinical-genomic classifiers have slightly worse performance than clinical only classifier in samples with small nodules. Clinical-genomic classifiers have better performance than clinical only classifier in samples with low/intermediate pre-test ROMs.

TABLE 29
Performances of classifiers, AUC of ROC

| | | | | Clinical | Clinical |
|---|---|---|---|---|---|
| | Clini- | Genomic | Genomic | Genomic | Genomic |
| Samples | cal | 1 | 2 | 1 | 2 |
| All (87 | 0.826 | 0.62 | 0.62 | 0.803 | 0.808 |
| benign, 203 | | | | | |
| malignant) | | | | | |
| Former | 0.833 | 0.602 | 0.595 | 0.824 | 0.818 |
| Smokers (55 | | | | | |
| benign, 109 | | | | | |
| malignant) | | | | | |
| Current | 0.824 | 0.629 | 0.642 | 0.771 | 0.798 |
| Smokers (32 | | | | | |
| benign, 94 | | | | | |
| malignant) | | | | | |

TABLE 30
Performances of classifiers, AUC of ROC

| | | | | Clinical | Clinical |
|---|---|---|---|---|---|
| | Clini- | Genomic | Genomic | Genomic | Genomic |
| Samples | cal | 1 | 2 | 1 | 2 |
| All (87 benign, | 0.826 | 0.62 | 0.62 | 0.803 | 0.808 |
| 203 malignant) | | | | | |
| Nodule size <3 | 0.833 | 0.635 | 0.667 | 0.813 | 0.817 |
| cm (38 benign, | | | | | |
| 72 malignant) | | | | | |
| Low/Interme- | 0.748 | 0.679 | 0.628 | 0.808 | 0.806 |
| diate pre-test | | | | | |
| ROM (71 benign, | | | | | |
| 25 malignant) | | | | | |

The clinical classifiers comprise input clinical factors: age, gender, smoking status, pack-year, years-since-quit, nodule length, and infiltrate nodule. The clinical classifiers were run with the following models: SVM, penalized GLM, and penalized GLM with interaction term.

The genomic classifiers comprise input from expression of genes chosen with various feature selection options and were run with the following models: SVM and penalized GLM.

The clinical-genomic classifiers comprise input clinical factors (age, gender, pack-year, years-since-quit, nodule length, infiltrate nodule) as well as genomic smoking status, and PIN. The clinical-genomic classifiers were run with the following models: SVM, penalized GLM, and penalized GLM with interaction terms.

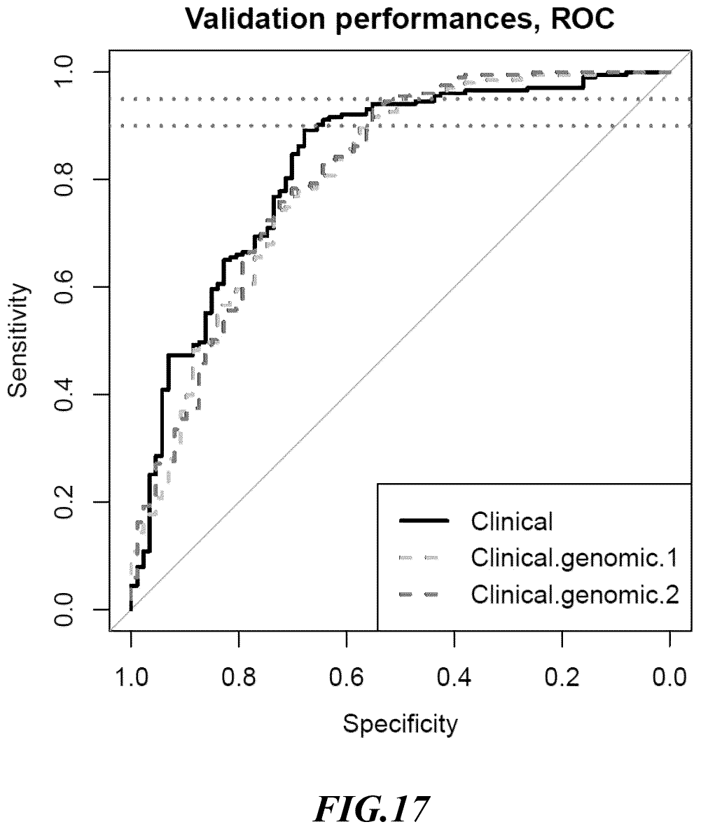

FIG. 17 is a graph of the validation performances, ROC, sensitivity v specificity, of the clinical only and clinical-genomic classifiers. The clinical-genomic classifiers performed better than clinical-only classifier in the very high sensitivity region of greater than or equal to 0.95.

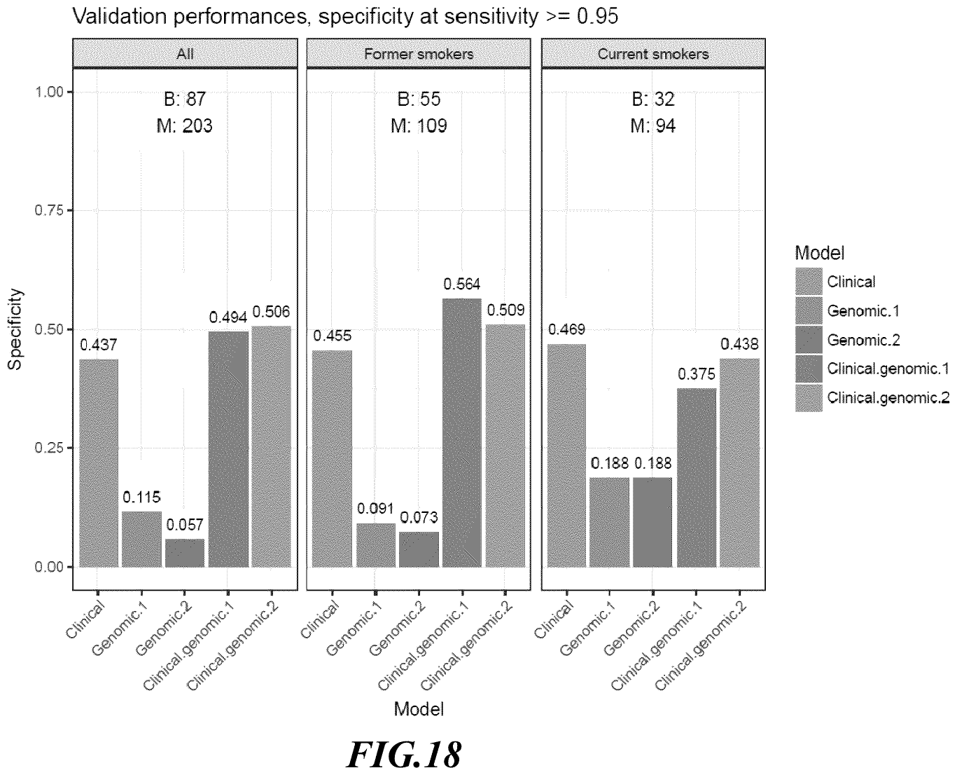

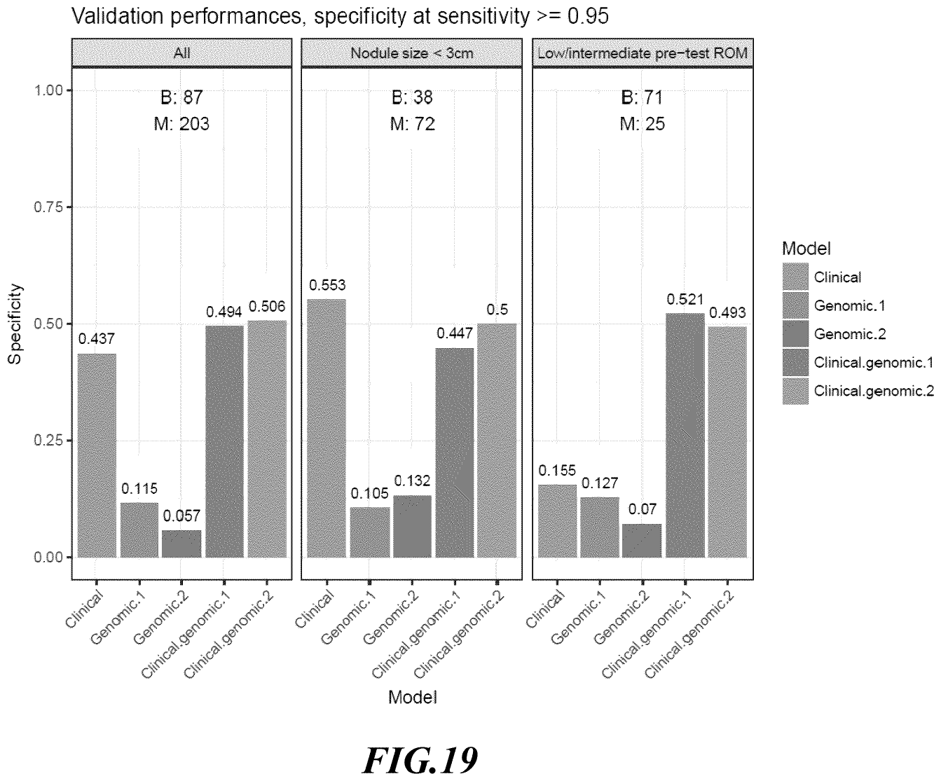

FIG. 18 and FIG. 19 show the specificity of the classifiers at a sensitivity greater than or equal to 0.95. Clinical-genomic classifiers have higher specificities than clinical only classifiers in all samples and in samples from former smokers only. Clinical genomic classifiers have higher specificities than clinical only classifiers in samples with low/intermediate pre-test ROMs. Table 21 and Table 22 below summarize the results:

TABLE 21
Specificity of the classifiers at a sensitivity
greater than or equal to 0.95

| | | | | Clinical | Clinical |
|---|---|---|---|---|---|
| | Clini- | Genomic | Genomic | Genomic | Genomic |
| Samples | cal | 1 | 2 | 1 | 2 |
| All (87 | 0.437 | 0.115 | 0.057 | 0.494 | 0.506 |
| benign, 203 | | | | | |
| malignant) | | | | | |
| Former | 0.455 | 0.091 | 0.073 | 0.564 | 0.509 |
| Smokers (55 | | | | | |
| benign, 109 | | | | | |
| malignant) | | | | | |
| Current | 0.469 | 0.188 | 0.188 | 0.375 | 0.438 |
| smokers (32 | | | | | |
| benign, 94 | | | | | |
| malignant) | | | | | |

TABLE 22
Specificity of the classifiers at a sensitivity
greater than or equal to 0.95

| | | | | Clinical | Clinical |
|---|---|---|---|---|---|
| | Clini- | Genomic | Genomic | Genomic | Genomic |
| Samples | cal | 1 | 2 | 1 | 2 |
| All (87 benign, | 0.437 | 0.115 | 0.057 | 0.494 | 0.506 |
| 203 malignant) | | | | | |
| Nodule size <3 | 0.553 | 0.105 | 0.132 | 0.447 | 0.5 |
| cm (38 benign, | | | | | |
| 72 malignant) | | | | | |
| Low/Interme- | 0.155 | 0.127 | 0.07 | 0.521 | 0.493 |
| diate pre-test | | | | | |
| ROM (71 benign, | | | | | |
| 25 malignant) | | | | | |

### Example 5: Training and Validation of an Algorithm to Determine Smoking Status by Gene Expression from Nasal Epithelial Cells

To further validate the classifiers, samples were randomly assigned to the training set and the validation set with a ratio of 3:2. Only samples with a RIN greater than or equal to 3 were used. The classifiers were built with the same five sets of options as seen above and in Examples 3 and 4. Table 12 below shows the number of nasal brushing samples from subjects diagnosed with benign or malignant tumors in the training and validation sample sets.

TABLE 12
Number of training and validation test samples

| | Cancer Diagnosis: | Cancer Diagnosis: | |
|---|---|---|---|
| Set | Benign | Malignant | Total |
| Training | 85 | 326 | 411 |
| Validation | 57 | 207 | 264 |
| Total | 142 | 533 | 675 |

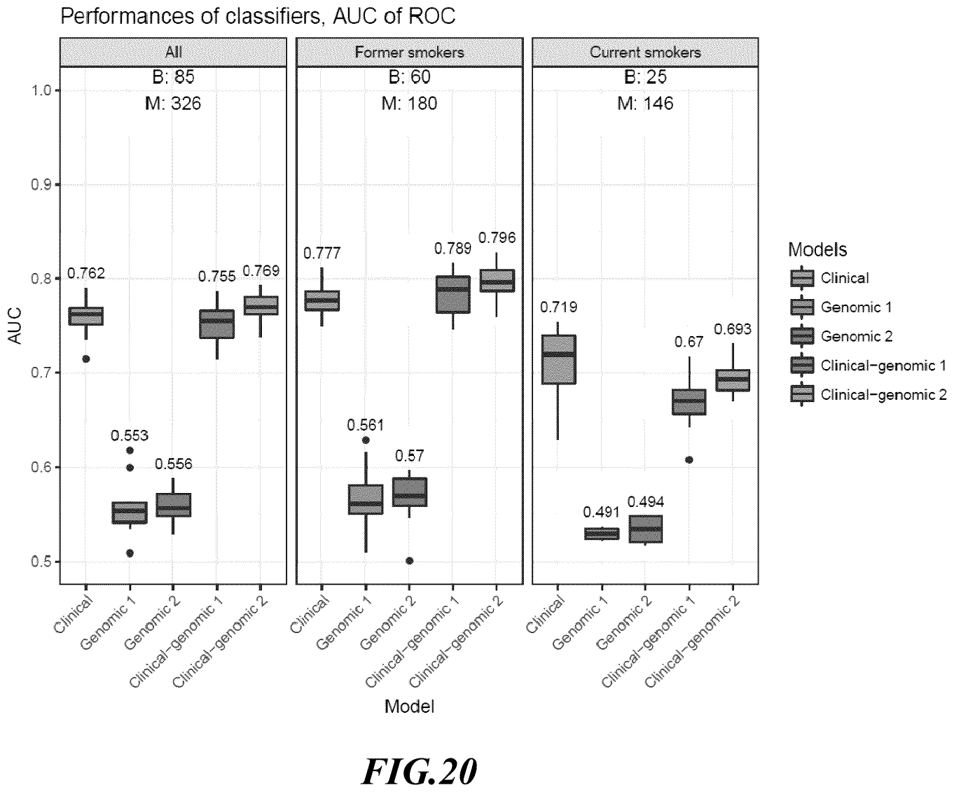

FIG. 20 is a graph showing the training performance of the five classifiers (clinical only, genomic 1, genomic 2, clinical-genomic 1 and clinical-genomic 2) that were used in Examples 3 and 4 as applied to the new training samples. The clinical-genomic classifiers have training performances similar to clinical only classifiers. Table 23 below summarizes the results.

TABLE 23
Performances of classifiers, AUC of ROC

| | | | | Clinical | Clinical |
|---|---|---|---|---|---|
| | Clini- | Genomic | Genomic | Genomic | Genomic |
| Samples | cal | 1 | 2 | 1 | 2 |
| All (85 | 0.762 | 0.553 | 0.556 | 0.755 | 0.769 |
| benign, 326 | | | | | |
| malignant) | | | | | |
| Former | 0.777 | 0.561 | 0.57 | 0.789 | 0.796 |
| Smokers (60 | | | | | |
| benign, 180 | | | | | |
| malignant) | | | | | |
| Current | 0.719 | 0.491 | 0.494 | 0.67 | 0.693 |
| Smokers (25 | | | | | |
| benign, 146 | | | | | |
| malignant) | | | | | |

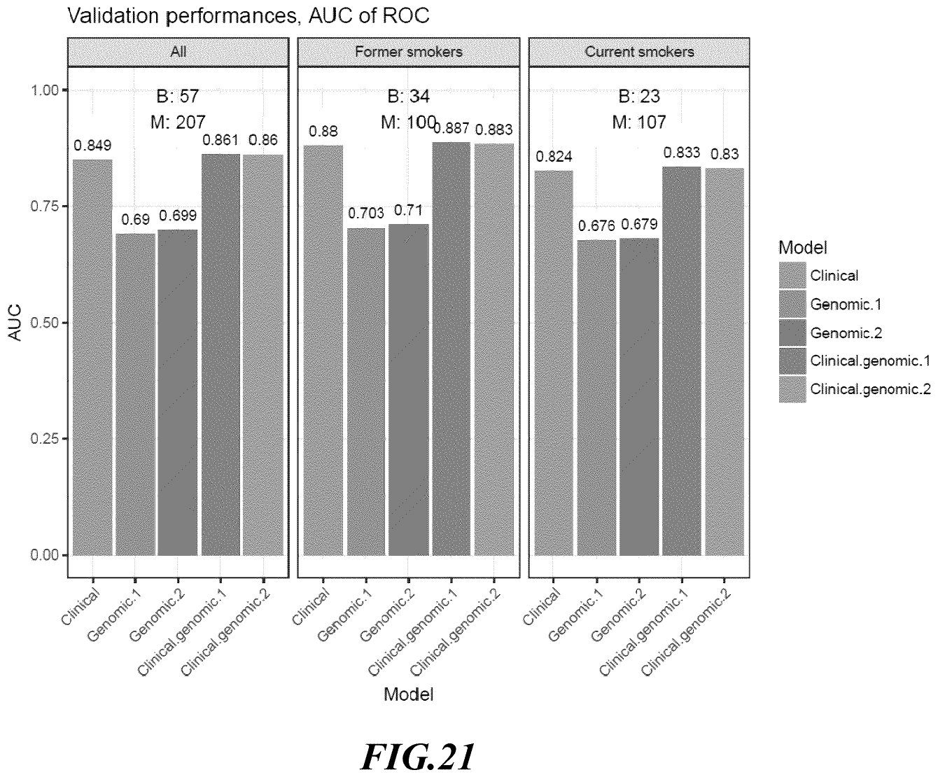

The classifiers were then validated using the new validation sample set. FIG. 21 shows the AUC of the classifiers. Clinical-genomic classifiers have better performance than clinical only classifiers. Table 24 below summarizes the results.

TABLE 24
Performances of classifiers, AUC of ROC

| | | | | Clinical | Clinical |
|---|---|---|---|---|---|
| | Clini- | Genomic | Genomic | Genomic | Genomic |
| Samples | cal | 1 | 2 | 1 | 2 |
| All (57 | 0.849 | 0.69 | 0.699 | 0.861 | 0.86 |
| benign, 207 | | | | | |
| malignant) | | | | | |
| Former | 0.88 | 0.703 | 0.71 | 0.887 | 0.883 |
| Smokers (34 | | | | | |
| benign, 100 | | | | | |
| malignant) | | | | | |
| Current | 0.824 | 0.676 | 0.679 | 0.833 | 0.83 |
| Smokers (23 | | | | | |
| benign, 107 | | | | | |
| malignant) | | | | | |

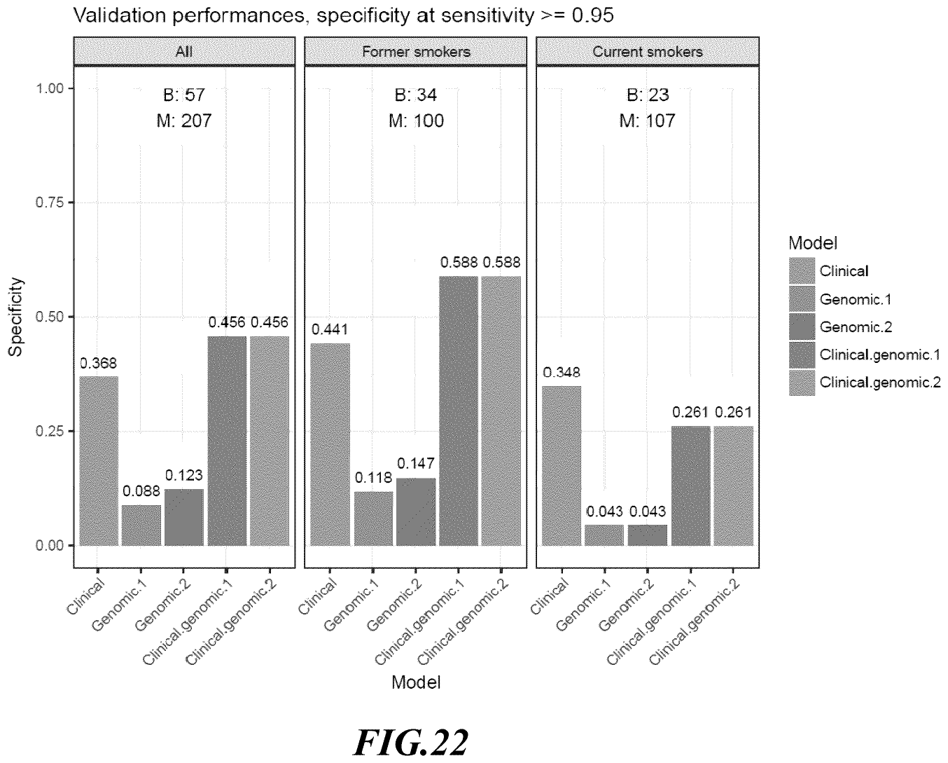

FIG. 22 shows the specificity of the classifiers at a sensitivity greater than or equal to 0.95. Clinical-genomic classifiers have higher specificities than clinical only classifiers in samples from former smokers only. Table 25 below summarizes the results.

TABLE 25
Validation performances, specificity at
sensitivity greater than or equal to 0.95

| | | | | Clinical | Clinical |
|---|---|---|---|---|---|
| | Clini- | Genomic | Genomic | Genomic | Genomic |
| Samples | cal | 1 | 2 | 1 | 2 |
| All (57 | 0.368 | 0.088 | 0.123 | 0.456 | 0.456 |
| benign, 207 | | | | | |
| malignant) | | | | | |
| Former | 0.441 | 0.118 | 0.147 | 0.588 | 0.588 |
| Smokers (34 | | | | | |
| benign, 100 | | | | | |
| malignant) | | | | | |
| Current | 0.348 | 0.043 | 0.043 | 0.261 | 0.261 |
| Smokers (23 | | | | | |
| benign, 107 | | | | | |
| malignant) | | | | | |

### Example 6: Reclassification of a Risk of Malignancy in Patients with Lung Nodules after a Nondiagnostic Bronchoscopy

Individuals who currently smoke or formerly smoked with an indeterminate lung nodule and a non-diagnostic bronchoscopy from the AEGIS I and II cohorts and the Registry were included. All patients underwent two bronchial brushings from the right mainstem bronchus during clinically indicated bronchoscopy to obtain bronchial epithelial cells from which mRNA was collected to perform whole transcriptome sequencing. Using predefined thresholds, the sensitivity, specificity, and predictive values for both the rule-out and rule-in thresholds of testing were calculated.

412 patients with nodules with a 39.6% prevalence of malignancy were included. Twenty-nine percent of intermediate risk lung nodules were down-classified to low risk with a sensitivity of 90.6% and a 91.0% negative predictive value (NPV) and 12.2% of intermediate risk nodules were up-classified to high risk with a 94.1% specificity and a 65.4% positive predictive value (PPV). In addition, 54.5% of low-risk nodules were down-classified to very low risk with 100% sensitivity and >99% NPV and 27.3% of high-risk nodules were up-classified to very high risk with a specificity of 91.2% and a 91.5% PPV.

The classifier has a high sensitivity for malignancy when used as a rule-out test and high specificity for malignancy when used as a rule-in test. It improves the diagnostic performance of bronchoscopy. The high accuracy of risk re-classification may lead to improved management of lung nodules.

Patients with an indeterminate lung nodule who had a non-diagnostic bronchoscopy from three different cohorts were evaluated for inclusion. The Airway Epithelium Gene Expression In the Diagnosis of Lung Cancer cohorts (AEGIS I and II) were recruited as a part of multi-center prospective observational studies. Participants were included from 24 centers in the United States, Canada and Ireland (Table 31) if they currently smoke or formerly smoked and were undergoing bronchoscopy for evaluation of lung nodules. The Registry cohort was a multi-center prospective registry that included patients with lung nodules who underwent clinically indicated diagnostic bronchoscopy at 34 medical centers across the US (Table 32). Institutional review board (IRB) approval was obtained by each institution before enrollment and informed consent was obtained from all patients. Two bronchial brushings were performed during bronchoscopy, and mRNA was collected from bronchial epithelial cells from the right mainstem bronchus. Before bronchoscopy, physicians assessed the pre-test risk of malignancy (ROM) for each patient, designated as low (<10%), intermediate (10-60%), or high (>60%) (5). Physicians could assign this assessment based on their clinical expertise or by using a published lung nodule risk model. Study personnel recorded nodule characteristics from the site radiologist report at each institution. All patients were followed for at least 12 months after bronchoscopy unless a diagnosis of malignancy was confirmed.

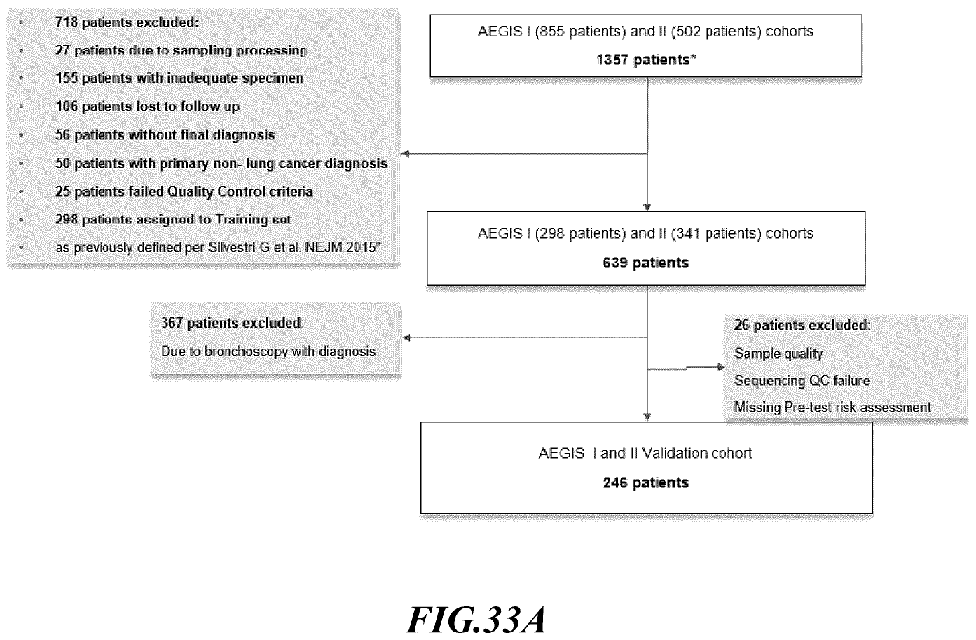

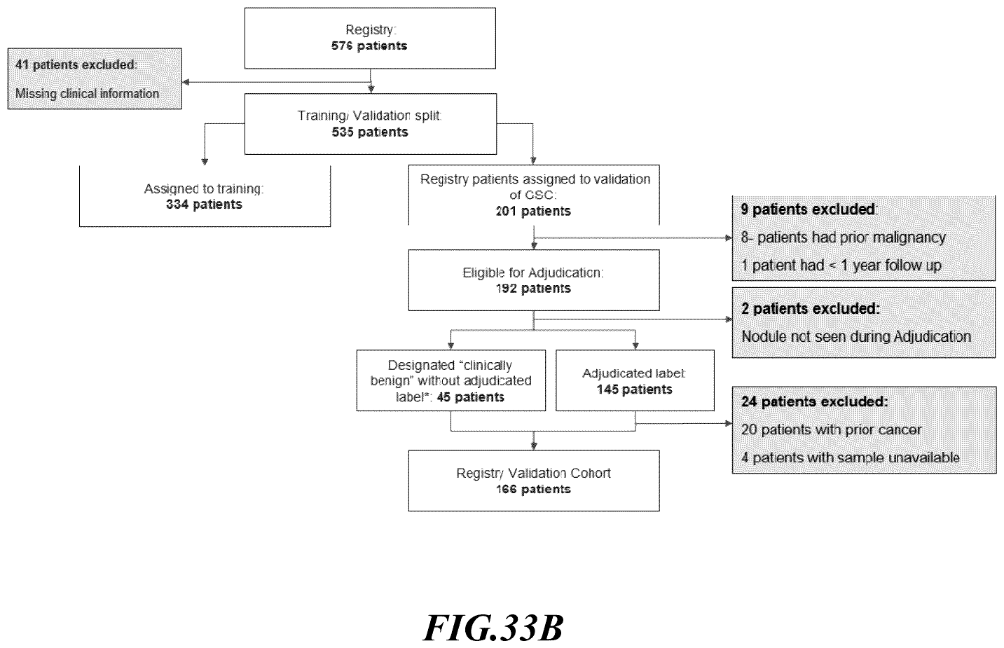

Patients from the AEGIS cohorts and the Registry were randomly split into a training cohort and a validation cohort (FIGS. 33A and 33B). The previously described algorithm development process was restricted to the training cohort. The algorithm development team was blinded to the validation cohort. After the final algorithm was locked, the performance of the classifier was determined by an unblinded third party. Only patients with a nodule suspicious for malignancy and a non-diagnostic bronchoscopy with at least one year follow up were included in this study. Exclusion criteria included age ≤21 years old, inability to provide informed consent, lack of tobacco use (smoked <100 cigarettes), or history of prior or concurrent cancer. All patients underwent an adjudication process, described below, to determine if the nodule was benign or malignant. Forty-five patients from the Registry who underwent adjudication and had stable imaging after 12 months but did not have a confirmed diagnosis by the adjudication rules were labeled “clinically benign” and excluded from the calculation of sensitivity and specificity of the GSC validation performance as they did not have individual truth labels. However, given the concern for significant bias of overestimation of cancer prevalence, these “clinically benign” nodules were included in calculating cancer prevalence. Since NPV, PPV, and risk re-classification are all functions of sensitivity, specificity, and cancer prevalence, these measures are impacted by these “clinically benign” patients through cancer prevalence.

A subset of patients was identified as having a diagnosis of chronic obstructive pulmonary disease (COPD) based upon the clinical expertise of the investigators at the time of enrollment. In addition to the overall accuracy assessment, the accuracy of the GSC was assessed for patients with and without COPD.

Diagnosis of a benign or malignant nodule was determined through an adjudication process. For the Registry Cohort, a live adjudication process was conducted to arbitrate a benign, malignant, or inconclusive consensus diagnosis by an expert 3-member pulmonologists panel. (HJL, DFK, LY). Panel members were provided with de-identified patient information with at least 12 months follow-up. Members of the panel were blinded to the GSC results.

A benign diagnosis was assigned in cases with 1) resolution of the nodule; 2) an alternative benign diagnosis; 3) nodule stability for ≥12 months and determination by the panel that the patient has no further suspicion of malignancy. Although two-year stability for radiographic imaging of nodules is recommended, this study included one-year stability of the nodule based upon prior studies that have found one-year nodule stability to be predictive of stability at two years (24, 28, 29). A malignant diagnosis was assigned in cases with pathology reports confirming malignancy, or a decision to treat a patient with stereotactic body radiation therapy (SBRT) without tissue confirmation.

To enhance confidence in the adjudication process, a subset of adjudicated patients underwent a second blinded independent central review by two independent oncologists with adjudication by a third oncologist, if needed. Reviewers were provided with the same clinical information as provided in the first adjudication process. Results were 95% concordant (Cohen's kappa=0.88), therefore data from the first adjudication was used for analysis.

The adjudication process for the AEGIS I and II cohorts was performed as previously described.

Two bronchial brush specimens were collected from the normal-appearing right mainstem bronchus during bronchoscopy, stored in a nucleic acid preservative (RNAprotect, QIAGEN, Hilden, Germany), then shipped (2-8C) to the testing laboratory. From each brushing sample, total RNA was extracted using the miRNeasy Mini Kit (QIAGEN, Hilden, Germany), quantitated (QuantiFluor RNA System, Promega, Madison, WI) and 50 ng was used as input to the TruSeq RNA Access Library Prep procedure (Illumina, San Diego, CA) for coding transcriptome enrichment. Libraries meeting quality control criteria were sequenced using NextSeq 500 instruments (2×75 bp paired-end reads) with the High Output Kit (Illumina, San Diego, CA). Raw sequencing (FASTQ) files were aligned to the Human Reference assembly 37 (Genome Reference Consortium) using the STAR RNA-seq aligner software. Uniquely mapped and non-duplicate reads were summarized for 63,677 annotated Ensembl genes using HTSeq. Data quality metrics were generated using RNA-SeQC. Samples were excluded and re-sequenced when their library sequence data did not achieve minimum criteria for total reads, uniquely mapped reads, mean per-base coverage, base duplication rate, percentage of bases aligned to coding regions, base mismatch rate, and uniformity of coverage within each gene.

GSC Algorithm Development

Normalization and gene filtering of the genomic sequencing data and the derivation of the algorithm of the GSC in the training cohort was previously described. The final ensemble score from the GSC algorithm is the logit of mean probabilities from four individual models. Together, the final ensemble classifier includes five clinical features (age, gender, pack-year, inhaled medication use, and specimen collection timing) and 1,232 gene features as listed in Table 37. This final ensemble classifier was developed and prospectively locked on a prior training cohort. The final ensemble classifier has pre-defined locked thresholds for risk-reclassification in the respective ROM groups.

TABLE 37
GSC gene features

| | |
|---|---|
| ENSG00000184389 | A3GALT2 |
| ENSG00000144452 | ABCA12 |
| ENSG00000073734 | ABCB11 |
| ENSG00000255277 | ABCC6P2 |
| ENSG00000248487 | ABHD14A |
| ENSG00000214841 | AC005493.1 |
| ENSG00000267090 | AC005789.9 |
| ENSG00000227407 | AC008746.3 |
| ENSG00000238045 | AC009133.14 |
| ENSG00000224361 | AC011239.1 |
| ENSG00000267896 | AC018766.4 |
| ENSG00000269352 | AC018766.5 |
| ENSG00000215067 | AC027763.2 |
| ENSG00000269657 | AC079210.1 |
| ENSG00000111271 | ACAD10 |
| ENSG00000151498 | ACAD8 |
| ENSG00000135847 | ACBD6 |
| ENSG00000131473 | ACLY |
| ENSG00000176715 | ACSF3 |
| ENSG00000139567 | ACVRL1 |
| ENSG00000196839 | ADA |
| ENSG00000229186 | ADAM1A |
| ENSG00000114948 | ADAM23 |
| ENSG00000042980 | ADAM28 |
| ENSG00000151388 | ADAMTS12 |
| ENSG00000138316 | ADAMTS14 |
| ENSG00000170214 | ADRA1B |
| ENSG00000150594 | ADRA2A |
| ENSG00000130706 | ADRM1 |
| ENSG00000185100 | ADSSL1 |
| ENSG00000223959 | AFG3L1P |
| ENSG00000183077 | AFMID |
| ENSG00000255737 | AGAP2-AS1 |
| ENSG00000204305 | AGER |
| ENSG00000135744 | AGT |
| ENSG00000063438 | AHRR |
| ENSG00000186063 | AIDA |
| ENSG00000183773 | AIFM3 |
| ENSG00000196581 | AJAP1 |
| ENSG00000227471 | AKR1B15 |
| ENSG00000165092 | ALDH1A1 |
| ENSG00000136010 | ALDH1L2 |
| ENSG00000119711 | ALDH6A1 |
| ENSG00000253981 | ALG1L13P |
| ENSG00000073331 | ALPK1 |
| ENSG00000136383 | ALPK3 |
| ENSG00000162551 | ALPL |
| ENSG00000160593 | AMICA1 |
| ENSG00000145020 | AMT |
| ENSG00000214274 | ANG |
| ENSG00000013523 | ANGEL1 |
| ENSG00000154188 | ANGPT1 |
| ENSG00000145362 | ANK2 |
| ENSG00000088448 | ANKRD10 |
| ENSG00000076513 | ANKRD13A |
| ENSG00000159712 | ANKRD18CP |
| ENSG00000135976 | ANKRD36 |
| ENSG00000196912 | ANKRD36B |
| ENSG00000154945 | ANKRD40 |
| ENSG00000168096 | ANKS3 |
| ENSG00000131620 | ANO1 |
| ENSG00000237276 | ANO7P1 |
| ENSG00000185101 | ANO9 |
| ENSG00000248546 | ANP32C |
| ENSG00000138279 | ANXA7 |
| ENSG00000131480 | AOC2 |
| ENSG00000131471 | AOC3 |
| ENSG00000138356 | AOX1 |
| ENSG00000243627 | AP000322.53 |
| ENSG00000254614 | AP003068.23 |
| ENSG00000213983 | AP1G2 |
| ENSG00000129354 | AP1M2 |
| ENSG00000134262 | AP4B1 |
| ENSG00000011132 | APBA3 |
| ENSG00000113108 | APBB3 |
| ENSG00000154856 | APCDD1 |
| ENSG00000163382 | APOA1BP |
| ENSG00000084674 | APOB |
| ENSG00000142192 | APP |
| ENSG00000136044 | APPL2 |
| ENSG00000186635 | ARAP1 |
| ENSG00000205595 | AREGB |
| ENSG00000134884 | ARGLU1 |
| ENSG00000075884 | ARHGAP15 |
| ENSG00000163219 | ARHGAP25 |
| ENSG00000145819 | ARHGAP26 |
| ENSG00000186517 | ARHGAP30 |
| ENSG00000089820 | ARHGAP4 |
| ENSG00000074964 | ARHGEF10L |
| ENSG00000114790 | ARHGEF26 |
| ENSG00000165801 | ARHGEF40 |
| ENSG00000129675 | ARHGEF6 |
| ENSG00000131089 | ARHGEF9 |
| ENSG00000188042 | ARL4C |
| ENSG00000241685 | ARPC1A |
| ENSG00000128989 | ARPP19 |
| ENSG00000100628 | ASB2 |
| ENSG00000110881 | ASIC1 |
| ENSG00000196433 | ASMT |
| ENSG00000236017 | ASMTL-AS1 |
| ENSG00000198356 | ASNA1 |
| ENSG00000123268 | ATF1 |
| ENSG00000168010 | ATG16L2 |
| ENSG00000197548 | ATG7 |
| ENSG00000142102 | ATHL1 |
| ENSG00000068650 | ATP11A |
| ENSG00000075673 | ATP12A |
| ENSG00000163399 | ATP1A1 |
| ENSG00000166377 | ATP9B |
| ENSG00000126895 | AVPR2 |
| ENSG00000160862 | AZGP1 |
| ENSG00000172232 | AZU1 |
| ENSG00000112208 | BAG2 |
| ENSG00000151929 | BAG3 |
| ENSG00000166170 | BAG5 |
| ENSG00000135298 | BAI3 |
| ENSG00000095739 | BAMBI |
| ENSG00000153064 | BANK1 |
| ENSG00000172530 | BANP |
| ENSG00000075790 | BCAP29 |
| ENSG00000060982 | BCAT1 |
| ENSG00000171552 | BCL2L1 |
| ENSG00000258643 | BCL2L2-PABPN1 |
| ENSG00000106635 | BCL7B |
| ENSG00000116128 | BCL9 |
| ENSG00000100739 | BDKRB1 |
| ENSG00000102409 | BEX4 |
| ENSG00000197299 | BLM |
| ENSG00000104081 | BMF |
| ENSG00000125378 | BMP4 |
| ENSG00000176171 | BNIP3 |
| ENSG00000163170 | BOLA3 |
| ENSG00000078898 | BPIFB2 |
| ENSG00000167104 | BPIFB6 |
| ENSG00000139618 | BRCA2 |
| ENSG00000166164 | BRD7 |
| ENSG00000113460 | BRIX1 |
| ENSG00000109743 | BST1 |
| ENSG00000112763 | BTN2A1 |
| ENSG00000124508 | BTN2A2 |
| ENSG00000124549 | BTN2A3P |
| ENSG00000204161 | C10orf128 |
| ENSG00000168070 | C11orf85 |
| ENSG00000257242 | C12orf79 |
| ENSG00000087302 | C14orf166 |
| ENSG00000186073 | C15orf41 |
| ENSG00000166920 | C15orf48 |
| ENSG00000130731 | C16orf13 |
| ENSG00000103544 | C16orf62 |
| ENSG00000172653 | C17orf66 |
| ENSG00000177025 | C19orf18 |
| ENSG00000118292 | C1orf54 |
| ENSG00000108561 | C1QBP |
| ENSG00000172247 | C1QTNF4 |
| ENSG00000222018 | C21orf140 |
| ENSG00000189269 | C22orf43 |
| ENSG00000173557 | C2orf70 |
| ENSG00000188315 | C3orf62 |
| ENSG00000123843 | C4BPB |
| ENSG00000138658 | C4orf21 |
| ENSG00000134830 | C5AR2 |
| ENSG00000185127 | C6orf120 |
| ENSG00000203872 | C6orf163 |
| ENSG00000021852 | C8B |
| ENSG00000136819 | C9orf78 |
| ENSG00000154975 | CA10 |
| ENSG00000074410 | CA12 |
| ENSG00000185015 | CA13 |
| ENSG00000178538 | CA8 |
| ENSG00000196557 | CACNA1H |
| ENSG00000157445 | CACNA2D3 |
| ENSG00000108878 | CACNG1 |
| ENSG00000075461 | CACNG4 |
| ENSG00000198668 | CALM1 |
| ENSG00000145349 | CAMK2D |
| ENSG00000092529 | CAPN3 |
| ENSG00000204397 | CARD16 |
| ENSG00000105483 | CARD8 |
| ENSG00000187796 | CARD9 |
| ENSG00000153048 | CARHSP1 |
| ENSG00000003400 | CASP10 |
| ENSG00000106144 | CASP2 |
| ENSG00000153113 | CAST |
| ENSG00000205771 | CATSPER2P1 |
| ENSG00000110395 | CBL |
| ENSG00000104957 | CCDC130 |
| ENSG00000128596 | CCDC136 |
| ENSG00000197599 | CCDC154 |
| ENSG00000163749 | CCDC158 |
| ENSG00000149201 | CCDC81 |
| ENSG00000168071 | CCDC88B |
| ENSG00000205021 | CCL3L1 |
| ENSG00000135083 | CCNJL |
| ENSG00000183625 | CCR3 |
| ENSG00000183813 | CCR4 |
| ENSG00000126353 | CCR7 |
| ENSG00000134256 | CD101 |
| ENSG00000135535 | CD164 |
| ENSG00000204936 | CD177 |
| ENSG00000177455 | CD19 |
| ENSG00000185275 | CD24P4 |
| ENSG00000178562 | CD28 |
| ENSG00000167850 | CD300C |
| ENSG00000102245 | CD40LG |
| ENSG00000143119 | CD53 |
| ENSG00000114013 | CD86 |
| ENSG00000002586 | CD99 |
| ENSG00000185324 | CDK10 |
| ENSG00000108465 | CDK5RAP3 |
| ENSG00000008086 | CDKL5 |
| ENSG00000168564 | CDKN2AIP |
| ENSG00000123080 | CDKN2C |
| ENSG00000184258 | CDR1 |
| ENSG00000170956 | CEACAM3 |
| ENSG00000007306 | CEACAM7 |
| ENSG00000099954 | CECR2 |
| ENSG00000123219 | CENPK |
| ENSG00000102901 | CENPT |
| ENSG00000166582 | CENPV |
| ENSG00000143418 | CERS2 |
| ENSG00000172828 | CES3 |
| ENSG00000087237 | CETP |
| ENSG00000243649 | CFB |
| ENSG00000135346 | CGA |
| ENSG00000138028 | CGREF1 |
| ENSG00000100532 | CGRRF1 |
| ENSG00000136457 | CHAD |
| ENSG00000186940 | CHCHD2P9 |
| ENSG00000170004 | CHD3 |
| ENSG00000072609 | CHFR |
| ENSG00000168539 | CHRM1 |
| ENSG00000175344 | CHRNA7 |
| ENSG00000170175 | CHRNB1 |
| ENSG00000198108 | CHSY3 |
| ENSG00000179583 | CIITA |
| ENSG00000198894 | CIPC |
| ENSG00000230055 | CISD3 |
| ENSG00000217555 | CKLF |
| ENSG00000171217 | CLDN20 |
| ENSG00000177300 | CLDN22 |
| ENSG00000132514 | CLEC10A |
| ENSG00000105472 | CLEC11A |
| ENSG00000111729 | CLEC4A |
| ENSG00000166523 | CLEC4E |
| ENSG00000115295 | CLIP4 |
| ENSG00000104853 | CLPTM1 |
| ENSG00000139182 | CLSTN3 |
| ENSG00000184220 | CMSS1 |
| ENSG00000153551 | CMTM7 |
| ENSG00000169714 | CNBP |
| ENSG00000108797 | CNTNAP1 |
| ENSG00000106078 | COBL |
| ENSG00000204248 | COL11A2 |
| ENSG00000082293 | COL19A1 |
| ENSG00000081052 | COL4A4 |
| ENSG00000230524 | COL6A4P1 |
| ENSG00000206384 | COL6A6 |
| ENSG00000049089 | COL9A2 |
| ENSG00000168090 | COPS6 |
| ENSG00000167549 | CORO6 |
| ENSG00000115944 | COX7A2L |
| ENSG00000160111 | CPAMD8 |
| ENSG00000109472 | CPE |
| ENSG00000140848 | CPNE2 |
| ENSG00000196353 | CPNE4 |
| ENSG00000178773 | CPNE7 |
| ENSG00000139117 | CPNE8 |
| ENSG00000021826 | CPS1 |
| ENSG00000146592 | CREB5 |
| ENSG00000150938 | CRIM1 |
| ENSG00000146215 | CRIP3 |
| ENSG00000006016 | CRLF1 |
| ENSG00000205755 | CRLF2 |
| ENSG00000095713 | CRTAC1 |
| ENSG00000139631 | CSAD |
| ENSG00000164400 | CSF2 |
| ENSG00000147408 | CSGALNACT1 |
| ENSG00000164796 | CSMD3 |
| ENSG00000175183 | CSRP2 |
| ENSG00000214249 | CTAGE11P |
| ENSG00000205041 | CTC-425O23.2 |
| ENSG00000259655 | CTD-2054N24.1 |
| ENSG00000266208 | CTD-2267D19.3 |
| ENSG00000258875 | CTD-2547L24.3 |
| ENSG00000267309 | CTD-2630F21.1 |
| ENSG00000188897 | CTD-3088G3.8 |
| ENSG00000107562 | CXCL12 |
| ENSG00000163464 | CXCR1 |
| ENSG00000180871 | CXCR2 |
| ENSG00000121966 | CXCR4 |
| ENSG00000138061 | CYP1B1 |
| ENSG00000186684 | CYP27C1 |
| ENSG00000197408 | CYP2B6 |
| ENSG00000256612 | CYP2B7P |
| ENSG00000100197 | CYP2D6 |
| ENSG00000205702 | CYP2D7P |
| ENSG00000130612 | CYP2G1P |
| ENSG00000233622 | CYP2T2P |
| ENSG00000155016 | CYP2U1 |
| ENSG00000186204 | CYP4F12 |
| ENSG00000186377 | CYP4X1 |
| ENSG00000186160 | CYP4Z1 |
| ENSG00000100055 | CYTH4 |
| ENSG00000115165 | CYTIP |
| ENSG00000165659 | DACH1 |
| ENSG00000204843 | DCTN1 |
| ENSG00000132912 | DCTN4 |
| ENSG00000153904 | DDAH1 |
| ENSG00000178404 | DDC8 |
| ENSG00000110367 | DDX6 |
| ENSG00000164825 | DEFB1 |
| ENSG00000100150 | DEPDC5 |
| ENSG00000099958 | DERL3 |
| ENSG00000153933 | DGKE |
| ENSG00000135829 | DHX9 |
| ENSG00000160305 | DIP2A |
| ENSG00000150768 | DLAT |
| ENSG00000132535 | DLG4 |
| ENSG00000104093 | DMXL2 |
| ENSG00000185842 | DNAH14 |
| ENSG00000187775 | DNAH17 |
| ENSG00000069345 | DNAJA2 |
| ENSG00000120675 | DNAJC15 |
| ENSG00000101152 | DNAJC5 |
| ENSG00000116675 | DNAJC6 |
| ENSG00000163687 | DNASE1L3 |
| ENSG00000119772 | DNMT3A |
| ENSG00000272636 | DOC2B |
| ENSG00000168631 | DPCR1 |
| ENSG00000184845 | DRD1 |
| ENSG00000134769 | DTNA |
| ENSG00000088986 | DYNLL1 |
| ENSG00000125971 | DYNLRB1 |
| ENSG00000147654 | EBAG9 |
| ENSG00000117395 | EBNA1BP2 |
| ENSG00000121310 | ECHDC2 |
| ENSG00000164176 | EDIL3 |
| ENSG00000101210 | EEF1A2 |
| ENSG00000159658 | EFCAB14 |
| ENSG00000176927 | EFCAB5 |
| ENSG00000138798 | EGF |
| ENSG00000173442 | EHBP1L1 |
| ENSG00000100353 | EIF3D |
| ENSG00000110321 | EIF4G2 |
| ENSG00000106682 | EIF4H |
| ENSG00000100664 | EIF5 |
| ENSG00000066044 | ELAVL1 |
| ENSG00000166897 | ELFN2 |
| ENSG00000115459 | ELMOD3 |
| ENSG00000170571 | EMB |
| ENSG00000231752 | EMBP1 |
| ENSG00000268758 | EMR4P |
| ENSG00000173818 | ENDOV |
| ENSG00000120658 | ENOX1 |
| ENSG00000112796 | ENPP5 |
| ENSG00000138185 | ENTPD1 |
| ENSG00000188833 | ENTPD8 |
| ENSG00000163378 | EOGT |
| ENSG00000166947 | EPB42 |
| ENSG00000105131 | EPHX3 |
| ENSG00000198758 | EPS8L3 |
| ENSG00000065361 | ERBB3 |
| ENSG00000104714 | ERICH1 |
| ENSG00000089248 | ERP29 |
| ENSG00000196405 | EVL |
| ENSG00000182473 | EXOC7 |
| ENSG00000162894 | FAIM3 |
| ENSG00000162636 | FAM102B |
| ENSG00000152102 | FAM168B |
| ENSG00000198780 | FAM169A |
| ENSG00000144369 | FAM171B |
| ENSG00000174132 | FAM174A |
| ENSG00000185442 | FAM174B |
| ENSG00000197520 | FAM177B |
| ENSG00000146067 | FAM193B |
| ENSG00000124103 | FAM209A |
| ENSG00000204930 | FAM221B |
| ENSG00000225828 | FAM229A |
| ENSG00000154511 | FAM69A |
| ENSG00000148343 | FAM73B |
| ENSG00000101447 | FAM83D |
| ENSG00000005812 | FBXL3 |
| ENSG00000156804 | FBXO32 |
| ENSG00000165355 | FBXO33 |
| ENSG00000177294 | FBXO39 |
| ENSG00000198019 | FCGR1B |
| ENSG00000143226 | FCGR2A |
| ENSG00000162747 | FCGR3B |
| ENSG00000130475 | FCHO1 |
| ENSG00000137478 | FCHSD2 |
| ENSG00000132704 | FCRL2 |
| ENSG00000088340 | FER1L4 |
| ENSG00000182511 | FES |
| ENSG00000128610 | FEZF1 |
| ENSG00000102466 | FGF14 |
| ENSG00000213066 | FGFR1OP |
| ENSG00000160867 | FGFR4 |
| ENSG00000000938 | FGR |
| ENSG00000137460 | FHDC1 |
| ENSG00000189283 | FHIT |
| ENSG00000134775 | FHOD3 |
| ENSG00000172500 | FIBP |
| ENSG00000214253 | FIS1 |
| ENSG00000162076 | FLYWCH2 |
| ENSG00000052795 | FNIP2 |
| ENSG00000171051 | FPR1 |
| ENSG00000156869 | FRRS1 |
| ENSG00000075539 | FRYL |
| ENSG00000188738 | FSIP2 |
| ENSG00000165775 | FUNDC2 |
| ENSG00000148803 | FUOM |
| ENSG00000128683 | GAD1 |
| ENSG00000179271 | GADD45GIP1 |
| ENSG00000144278 | GALNT13 |
| ENSG00000115339 | GALNT3 |
| ENSG00000213930 | GALT |
| ENSG00000214013 | GANC |
| ENSG00000139354 | GAS2L3 |
| ENSG00000162645 | GBP2 |
| ENSG00000154451 | GBP5 |
| ENSG00000203879 | GDI1 |
| ENSG00000178795 | GDPD4 |
| ENSG00000158555 | GDPD5 |
| ENSG00000168827 | GFM1 |
| ENSG00000146013 | GFRA3 |
| ENSG00000115486 | GGCX |
| ENSG00000100121 | GGTLC2 |
| ENSG00000183038 | GGTLC3 |
| ENSG00000139436 | GIT2 |
| ENSG00000187513 | GJA4 |
| ENSG00000198814 | GK |
| ENSG00000090863 | GLG1 |
| ENSG00000156689 | GLYATL2 |
| ENSG00000168237 | GLYCTK |
| ENSG00000140632 | GLYR1 |
| ENSG00000130755 | GMFG |
| ENSG00000137198 | GMPR |
| ENSG00000088256 | GNA11 |
| ENSG00000168243 | GNG4 |
| ENSG00000111670 | GNPTAB |
| ENSG00000147437 | GNRH1 |
| ENSG00000184206 | GOLGA6L4 |
| ENSG00000175265 | GOLGA8A |
| ENSG00000113384 | GOLPH3 |
| ENSG00000116580 | GON4L |
| ENSG00000169347 | GP2 |
| ENSG00000143167 | GPA33 |
| ENSG00000149735 | GPHA2 |
| ENSG00000077585 | GPR137B |
| ENSG00000154165 | GPR15 |
| ENSG00000184194 | GPR173 |
| ENSG00000169508 | GPR183 |
| ENSG00000183840 | GPR39 |
| ENSG00000140030 | GPR65 |
| ENSG00000166123 | GPT2 |
| ENSG00000166923 | GREM1 |
| ENSG00000163873 | GRIK3 |
| ENSG00000152822 | GRM1 |
| ENSG00000168959 | GRM5 |
| ENSG00000186088 | GSAP |
| ENSG00000174156 | GSTA3 |
| ENSG00000213366 | GSTM2 |
| ENSG00000084207 | GSTP1 |
| ENSG00000122034 | GTF3A |
| ENSG00000148308 | GTF3C5 |
| ENSG00000204529 | GUCY2EP |
| ENSG00000138796 | HADH |
| ENSG00000112855 | HARS2 |
| ENSG00000244734 | HBB |
| ENSG00000255398 | HCAR3 |
| ENSG00000111906 | HDDC2 |
| ENSG00000166503 | HDGFRP3 |
| ENSG00000130021 | HDHD1 |
| ENSG00000162639 | HENMT1 |
| ENSG00000188290 | HES4 |
| ENSG00000213614 | HEXA |
| ENSG00000169660 | HEXDC |
| ENSG00000135547 | HEY2 |
| ENSG00000124440 | HIF3A |
| ENSG00000110422 | HIPK3 |
| ENSG00000198339 | HIST1H41 |
| ENSG00000156515 | HK1 |
| ENSG00000204257 | HLA-DMA |
| ENSG00000242574 | HLA-DMB |
| ENSG00000204252 | HLA-DOA |
| ENSG00000223865 | HLA-DPB1 |
| ENSG00000196735 | HLA-DQA1 |
| ENSG00000232629 | HLA-DQB2 |
| ENSG00000204287 | HLA-DRA |
| ENSG00000204642 | HLA-F |
| ENSG00000204632 | HLA-G |
| ENSG00000136630 | HLX |
| ENSG00000148357 | HMCN2 |
| ENSG00000134240 | HMGCS2 |
| ENSG00000179362 | HMGN2P46 |
| ENSG00000100292 | HMOX1 |
| ENSG00000135100 | HNF1A |
| ENSG00000215271 | HOMEZ |
| ENSG00000095066 | HOOK2 |
| ENSG00000168172 | HOOK3 |
| ENSG00000164120 | HPGD |
| ENSG00000107521 | HPS1 |
| ENSG00000182601 | HS3ST4 |
| ENSG00000215769 | hsa-mir-6080 |
| ENSG00000087076 | HSD17B14 |
| ENSG00000130948 | HSD17B3 |
| ENSG00000119471 | HSDL2 |
| ENSG00000096384 | HSP90AB1 |
| ENSG00000242028 | HYPK |
| ENSG00000116237 | ICMT |
| ENSG00000117318 | ID3 |
| ENSG00000211895 | IGHA1 |
| ENSG00000211897 | IGHG3 |
| ENSG00000211941 | IGHV3-11 |
| ENSG00000211949 | IGHV3-23 |
| ENSG00000211970 | IGHV4-61 |
| ENSG00000211933 | IGHV6-1 |
| ENSG00000243290 | IGKV1-12 |
| ENSG00000240864 | IGKV1-16 |
| ENSG00000240834 | IGKV1D-12 |
| ENSG00000241244 | IGKV1D-16 |
| ENSG00000239951 | IGKV3-20 |
| ENSG00000211671 | IGLV2-8 |
| ENSG00000211667 | IGLV3-12 |
| ENSG00000117154 | IGSF21 |
| ENSG00000140749 | IGSF6 |
| ENSG00000104365 | IKBKB |
| ENSG00000143466 | IKBKE |
| ENSG00000137070 | IL11RA |
| ENSG00000168811 | IL12A |
| ENSG00000112115 | IL17A |
| ENSG00000188263 | IL17REL |
| ENSG00000016402 | IL20RA |
| ENSG00000110944 | IL23A |
| ENSG00000162594 | IL23R |
| ENSG00000147168 | IL2RG |
| ENSG00000125571 | IL37 |
| ENSG00000104432 | IL7 |
| ENSG00000169429 | IL8 |
| ENSG00000104331 | IMPAD1 |
| ENSG00000081148 | IMPG2 |
| ENSG00000122641 | INHBA |
| ENSG00000204084 | INPP5B |
| ENSG00000165458 | INPPL1 |
| ENSG00000248099 | INSL3 |
| ENSG00000171105 | INSR |
| ENSG00000065150 | IPO5 |
| ENSG00000259673 | IQCH-AS1 |
| ENSG00000090376 | IRAK3 |
| ENSG00000126456 | IRF3 |
| ENSG00000137265 | IRF4 |
| ENSG00000213928 | IRF9 |
| ENSG00000170549 | IRX1 |
| ENSG00000136003 | ISCU |
| ENSG00000161638 | ITGA5 |
| ENSG00000140678 | ITGAX |
| ENSG00000179914 | ITLN1 |
| ENSG00000137825 | ITPKA |
| ENSG00000099840 | IZUMO4 |
| ENSG00000188385 | JAKMIP3 |
| ENSG00000172977 | KAT5 |
| ENSG00000069424 | KCNAB2 |
| ENSG00000131398 | KCNC3 |
| ENSG00000120049 | KCNIP2 |
| ENSG00000134504 | KCTD1 |
| ENSG00000110906 | KCTD10 |
| ENSG00000100196 | KDELR3 |
| ENSG00000073614 | KDM5A |
| ENSG00000128052 | KDR |
| ENSG00000102445 | KIAA0226L |
| ENSG00000132680 | KIAA0907 |
| ENSG00000122203 | KIAA1191 |
| ENSG00000164323 | KIAA1430 |
| ENSG00000139116 | KIF21A |
| ENSG00000068796 | KIF2A |
| ENSG00000170759 | KIF5B |
| ENSG00000130487 | KLHDC7B |
| ENSG00000162873 | KLHDC8A |
| ENSG00000185909 | KLHDC8B |
| ENSG00000179454 | KLHL28 |
| ENSG00000146021 | KLHL3 |
| ENSG00000145332 | KLHL8 |
| ENSG00000167757 | KLK11 |
| ENSG00000114030 | KPNA1 |
| ENSG00000118162 | KPTN |
| ENSG00000147121 | KRBOX4 |
| ENSG00000186395 | KRT10 |
| ENSG00000111057 | KRT18 |
| ENSG00000172867 | KRT2 |
| ENSG00000171403 | KRT9 |
| ENSG00000115919 | KYNU |
| ENSG00000182866 | LCK |
| ENSG00000184925 | LCN12 |
| ENSG00000136167 | LCP1 |
| ENSG00000174106 | LEMD3 |
| ENSG00000167615 | LENG8 |
| ENSG00000116977 | LGALS8 |
| ENSG00000218357 | LL22NC03-75H12.2 |
| ENSG00000131899 | LLGL1 |
| ENSG00000105983 | LMBR1 |
| ENSG00000139636 | LMBR1L |
| ENSG00000162761 | LMX1A |
| ENSG00000167210 | LOXHD1 |
| ENSG00000150471 | LPHN3 |
| ENSG00000110031 | LPXN |
| ENSG00000183423 | LRIT3 |
| ENSG00000263142 | LRRC37A17P |
| ENSG00000148948 | LRRC4C |
| ENSG00000163428 | LRRC58 |
| ENSG00000188906 | LRRK2 |
| ENSG00000204482 | LST1 |
| ENSG00000226979 | LTA |
| ENSG00000227507 | LTB |
| ENSG00000007392 | LUC7L |
| ENSG00000154589 | LY96 |
| ENSG00000254087 | LYN |
| ENSG00000197353 | LYPD2 |
| ENSG00000083099 | LYRM2 |
| ENSG00000099949 | LZTR1 |
| ENSG00000088899 | LZTS3 |
| ENSG00000179222 | MAGED1 |
| ENSG00000198042 | MAK16 |
| ENSG00000196547 | MAN2A2 |
| ENSG00000109323 | MANBA |
| ENSG00000104814 | MAP4K1 |
| ENSG00000100030 | MAPK1 |
| ENSG00000138834 | MAPK8IP3 |
| ENSG00000166974 | MAPRE2 |
| ENSG00000127241 | MASP1 |
| ENSG00000180611 | MB21D2 |
| ENSG00000104738 | MCM4 |
| ENSG00000063322 | MED29 |
| ENSG00000081189 | MEF2C |
| ENSG00000112818 | MEP1A |
| ENSG00000105976 | MET |
| ENSG00000165792 | METTL17 |
| ENSG00000067365 | METTL22 |
| ENSG00000169026 | MFSD7 |
| ENSG00000261857 | MIA |
| ENSG00000154305 | MIA3 |
| ENSG00000204520 | MICA |
| ENSG00000204516 | MICB |
| ENSG00000101871 | MID1 |
| ENSG00000267195 | MIR212 |
| ENSG00000207939 | MIR223 |
| ENSG00000207698 | MIR32 |
| ENSG00000207932 | MIR33A |
| ENSG00000198995 | MIR340 |
| ENSG00000207562 | MIR34C |
| ENSG00000198976 | MIR429 |
| ENSG00000207728 | MIR449B |
| ENSG00000208002 | MIR643 |
| ENSG00000207579 | MIR662 |
| ENSG00000196549 | MME |
| ENSG00000163563 | MNDA |
| ENSG00000123562 | MORF4L2 |
| ENSG00000143158 | MPC2 |
| ENSG00000130830 | MPP1 |
| ENSG00000156968 | MPV17L |
| ENSG00000135324 | MRAP2 |
| ENSG00000179832 | MROH1 |
| ENSG00000117501 | MROH9 |
| ENSG00000143314 | MRPL24 |
| ENSG00000185608 | MRPL40 |
| ENSG00000143436 | MRPL9 |
| ENSG00000131368 | MRPS25 |
| ENSG00000112996 | MRPS30 |
| ENSG00000074071 | MRPS34 |
| ENSG00000173531 | MST1 |
| ENSG00000146410 | MTFR2 |
| ENSG00000163719 | MTMR14 |
| ENSG00000087053 | MTMR2 |
| ENSG00000102043 | MTMR8 |
| ENSG00000168412 | MTNR1A |
| ENSG00000173171 | MTX1 |
| ENSG00000169550 | MUC15 |
| ENSG00000215182 | MUC5AC |
| ENSG00000172551 | MUCL1 |
| ENSG00000059728 | MXD1 |
| ENSG00000266714 | MYO15B |
| ENSG00000041515 | MYO16 |
| ENSG00000166866 | MYO1A |
| ENSG00000174527 | MYO1H |
| ENSG00000137474 | MYO7A |
| ENSG00000120729 | MYOT |
| ENSG00000139597 | N4BP2L1 |
| ENSG00000138386 | NAB1 |
| ENSG00000136274 | NACAD |
| ENSG00000172890 | NADSYN1 |
| ENSG00000145414 | NAF1 |
| ENSG00000249437 | NAIP |
| ENSG00000067798 | NAV3 |
| ENSG00000144426 | NBEAL1 |
| ENSG00000163386 | NBPF10 |
| ENSG00000243452 | NBPF15 |
| ENSG00000203827 | NBPF16 |
| ENSG00000142794 | NBPF3 |
| ENSG00000061676 | NCKAP1 |
| ENSG00000102471 | NDFIP2 |
| ENSG00000151414 | NEK7 |
| ENSG00000184613 | NELL2 |
| ENSG00000162139 | NEU3 |
| ENSG00000214357 | NEURL1B |
| ENSG00000235568 | NFAM1 |
| ENSG00000100968 | NFATC4 |
| ENSG00000077150 | NFKB2 |
| ENSG00000167604 | NFKBID |
| ENSG00000146232 | NFKBIE |
| ENSG00000166681 | NGFRAP1 |
| ENSG00000188811 | NHLRC3 |
| ENSG00000145912 | NHP2 |
| ENSG00000100138 | NHP2L1 |
| ENSG00000184117 | NIPSNAP1 |
| ENSG00000167034 | NKX3-1 |
| ENSG00000174885 | NLRP6 |
| ENSG00000132911 | NMUR2 |
| ENSG00000225921 | NOL7 |
| ENSG00000166197 | NOLC1 |
| ENSG00000164867 | NOS3 |
| ENSG00000134250 | NOTCH2 |
| ENSG00000213240 | NOTCH2NL |
| ENSG00000139910 | NOVA1 |
| ENSG00000007952 | NOX1 |
| ENSG00000196408 | NOXO1 |
| ENSG00000015520 | NPC1L1 |
| ENSG00000159899 | NPR2 |
| ENSG00000165671 | NSD1 |
| ENSG00000169189 | NSMCE1 |
| ENSG00000076685 | NT5C2 |
| ENSG00000135778 | NTPCR |
| ENSG00000148053 | NTRK2 |
| ENSG00000155561 | NUP205 |
| ENSG00000124006 | OBSL1 |
| ENSG00000130558 | OLFM1 |
| ENSG00000196403 | OR10D1P |
| ENSG00000168158 | OR2C1 |
| ENSG00000180988 | OR52N2 |
| ENSG00000141447 | OSBPL1A |
| ENSG00000165899 | OTOGL |
| ENSG00000181631 | P2RY13 |
| ENSG00000174944 | P2RY14 |
| ENSG00000101104 | PABPC1L |
| ENSG00000076641 | PAG1 |
| ENSG00000128050 | PAICS |
| ENSG00000145730 | PAM |
| ENSG00000073150 | PANX2 |
| ENSG00000148832 | PAOX |
| ENSG00000121274 | PAPD5 |
| ENSG00000138801 | PAPSS1 |
| ENSG00000137817 | PARP6 |
| ENSG00000229474 | PATL2 |
| ENSG00000165194 | PCDH19 |
| ENSG00000120324 | PCDHB10 |
| ENSG00000177839 | PCDHB9 |
| ENSG00000253910 | PCDHGB2 |
| ENSG00000125851 | PCSK2 |
| ENSG00000169174 | PCSK9 |
| ENSG00000106244 | PDAP1 |
| ENSG00000172572 | PDE3A |
| ENSG00000131435 | PDLIM4 |
| ENSG00000165650 | PDZD8 |
| ENSG00000162366 | PDZK1IP1 |
| ENSG00000163218 | PGLYRP4 |
| ENSG00000079739 | PGM1 |
| ENSG00000102174 | PHEX |
| ENSG00000054148 | PHPT1 |
| ENSG00000006576 | PHTF2 |
| ENSG00000175309 | PHYKPL |
| ENSG00000124102 | PI3 |
| ENSG00000153823 | PID1 |
| ENSG00000124155 | PIGT |
| ENSG00000100100 | PIK3IP1 |
| ENSG00000141506 | PIK3R5 |
| ENSG00000085514 | PILRA |
| ENSG00000166908 | PIP4K2C |
| ENSG00000241878 | PISD |
| ENSG00000057757 | PITHD1 |
| ENSG00000057294 | PKP2 |
| ENSG00000123739 | PLA2G12A |
| ENSG00000011422 | PLAUR |
| ENSG00000124181 | PLCG1 |
| ENSG00000154822 | PLCL2 |
| ENSG00000182378 | PLCXD1 |
| ENSG00000106086 | PLEKHA8 |
| ENSG00000120278 | PLEKHG1 |
| ENSG00000090924 | PLEKHG2 |
| ENSG00000196155 | PLEKHG4 |
| ENSG00000054690 | PLEKHH1 |
| ENSG00000241839 | PLEKHO2 |
| ENSG00000147872 | PLIN2 |
| ENSG00000102007 | PLP2 |
| ENSG00000136040 | PLXNC1 |
| ENSG00000127957 | PMS2P3 |
| ENSG00000123965 | PMS2P5 |
| ENSG00000240694 | PNMA2 |
| ENSG00000006757 | PNPLA4 |
| ENSG00000014138 | POLA2 |
| ENSG00000106628 | POLD2 |
| ENSG00000148229 | POLE3 |
| ENSG00000102978 | POLR2C |
| ENSG00000105171 | POP4 |
| ENSG00000110777 | POU2AF1 |
| ENSG00000138621 | PPCDC |
| ENSG00000125534 | PPDPF |
| ENSG00000177380 | PPFIA3 |
| ENSG00000104695 | PPP2CB |
| ENSG00000074211 | PPP2R2C |
| ENSG00000138814 | PPP3CA |
| ENSG00000154845 | PPP4R1 |
| ENSG00000124224 | PPP4R1L |
| ENSG00000040487 | PQLC2 |
| ENSG00000133246 | PRAM1 |
| ENSG00000123131 | PRDX4 |
| ENSG00000108946 | PRKAR1A |
| ENSG00000114302 | PRKAR2A |
| ENSG00000126583 | PRKCG |
| ENSG00000183943 | PRKX |
| ENSG00000099725 | PRKY |
| ENSG00000132600 | PRMT7 |
| ENSG00000147471 | PROSC |
| ENSG00000110107 | PRPF19 |
| ENSG00000174231 | PRPF8 |
| ENSG00000147224 | PRPS1 |
| ENSG00000111215 | PRR4 |
| ENSG00000135362 | PRR5L |
| ENSG00000135378 | PRRG4 |
| ENSG00000167157 | PRRX2 |
| ENSG00000112812 | PRSS16 |
| ENSG00000005001 | PRSS22 |
| ENSG00000150687 | PRSS23 |
| ENSG00000146250 | PRSS35 |
| ENSG00000178226 | PRSS36 |
| ENSG00000215148 | PRSS41 |
| ENSG00000099341 | PSMD8 |
| ENSG00000183527 | PSMG1 |
| ENSG00000140368 | PSTPIP1 |
| ENSG00000073756 | PTGS2 |
| ENSG00000179295 | PTPN11 |
| ENSG00000204179 | PTPN20A |
| ENSG00000070159 | PTPN3 |
| ENSG00000213402 | PTPRCAP |
| ENSG00000155093 | PTPRN2 |
| ENSG00000177707 | PVRL3 |
| ENSG00000168994 | PXDC1 |
| ENSG00000119943 | PYROXD2 |
| ENSG00000145337 | PYURF |
| ENSG00000129646 | QRICH2 |
| ENSG00000167964 | RAB26 |
| ENSG00000109113 | RAB34 |
| ENSG00000197562 | RAB40C |
| ENSG00000168118 | RAB4A |
| ENSG00000166128 | RAB8B |
| ENSG00000123570 | RAB9B |
| ENSG00000136933 | RABEPK |
| ENSG00000179262 | RAD23A |
| ENSG00000119318 | RAD23B |
| ENSG00000170471 | RALGAPB |
| ENSG00000076864 | RAP1GAP |
| ENSG00000075391 | RASAL2 |
| ENSG00000105538 | RASIP1 |
| ENSG00000101265 | RASSF2 |
| ENSG00000162775 | RBM15 |
| ENSG00000100461 | RBM23 |
| ENSG00000004534 | RBM6 |
| ENSG00000179051 | RCC2 |
| ENSG00000100918 | REC8 |
| ENSG00000102032 | RENBP |
| ENSG00000174236 | REP15 |
| ENSG00000165731 | RET |
| ENSG00000237441 | RGL2 |
| ENSG00000116741 | RGS2 |
| ENSG00000117152 | RGS4 |
| ENSG00000129667 | RHBDF2 |
| ENSG00000173156 | RHOD |
| ENSG00000177181 | RIMKLA |
| ENSG00000176406 | RIMS2 |
| ENSG00000123091 | RNF11 |
| ENSG00000133874 | RNF122 |
| ENSG00000170153 | RNF150 |
| ENSG00000108523 | RNF167 |
| ENSG00000145428 | RNF175 |
| ENSG00000155827 | RNF20 |
| ENSG00000158286 | RNF207 |
| ENSG00000187147 | RNF220 |
| ENSG00000205937 | RNPS1 |
| ENSG00000154134 | ROBO3 |
| ENSG00000263271 | RP11-1055B8.8 |
| ENSG00000259772 | RP11-16E12.2 |
| ENSG00000269609 | RP11-18I14.10 |
| ENSG00000225032 | RP11-228B15.4 |
| ENSG00000187812 | RP11-24M17.5 |
| ENSG00000116883 | RP11-268J15.5 |
| ENSG00000262712 | RP11-295D4.1 |
| ENSG00000237188 | RP11-337C18.8 |
| ENSG00000272849 | RP11-347I19.8 |
| ENSG00000259649 | RP11-351M8.1 |
| ENSG00000250989 | RP11-392E22.5 |
| ENSG00000214796 | RP11-480I12.5 |
| ENSG00000206532 | RP11-553A10.1 |
| ENSG00000254761 | RP11-672A2.1 |
| ENSG00000272947 | RP11-71H17.9 |
| ENSG00000254461 | RP11-755F10.3 |
| ENSG00000251615 | RP11-774O3.3 |
| ENSG00000255093 | RP11-794P6.2 |
| ENSG00000254469 | RP11-849H4.2 |
| ENSG00000262222 | RP11-876N24.4 |
| ENSG00000236869 | RP11-944L7.4 |
| ENSG00000183638 | RP1L1 |
| ENSG00000238164 | RP3-395M20.8 |
| ENSG00000273137 | RP3-402G11.28 |
| ENSG00000225450 | RP3-508I15.14 |
| ENSG00000231663 | RP5-827C21.4 |
| ENSG00000117748 | RPA2 |
| ENSG00000153574 | RPIA |
| ENSG00000101413 | RPRD1B |
| ENSG00000163125 | RPRD2 |
| ENSG00000177519 | RPRM |
| ENSG00000179673 | RPRML |
| ENSG00000155876 | RRAGA |
| ENSG00000248124 | RRN3P1 |
| ENSG00000103472 | RRN3P2 |
| ENSG00000179041 | RRS1 |
| ENSG00000159579 | RSPRY1 |
| ENSG00000105784 | RUNDC3B |
| ENSG00000013392 | RWDD2A |
| ENSG00000163602 | RYBP |
| ENSG00000101115 | SALL4 |
| ENSG00000123453 | SARDH |
| ENSG00000130066 | SAT1 |
| ENSG00000151748 | SAV1 |
| ENSG00000085365 | SCAMP1 |
| ENSG00000074660 | SCARF1 |
| ENSG00000249784 | SCARNA22 |
| ENSG00000124939 | SCGB2A1 |
| ENSG00000144285 | SCN1A |
| ENSG00000166828 | SCNN1G |
| ENSG00000139410 | SDSL |
| ENSG00000214491 | SEC14L6 |
| ENSG00000138802 | SEC24B |
| ENSG00000075826 | SEC31B |
| ENSG00000065665 | SEC61A2 |
| ENSG00000008952 | SEC62 |
| ENSG00000174175 | SELP |
| ENSG00000075213 | SEMA3A |
| ENSG00000170381 | SEMA3E |
| ENSG00000138623 | SEMA7A |
| ENSG00000161956 | SENP3 |
| ENSG00000186910 | SERPINA11 |
| ENSG00000166396 | SERPINB7 |
| ENSG00000167711 | SERPINF2 |
| ENSG00000149131 | SERPING1 |
| ENSG00000168137 | SETD5 |
| ENSG00000099995 | SF3A1 |
| ENSG00000087365 | SF3B2 |
| ENSG00000143368 | SF3B4 |
| ENSG00000104332 | SFRP1 |
| ENSG00000145423 | SFRP2 |
| ENSG00000166224 | SGPL1 |
| ENSG00000141258 | SGSM2 |
| ENSG00000095370 | SH2D3C |
| ENSG00000214193 | SH3D21 |
| ENSG00000148341 | SH3GLB2 |
| ENSG00000147010 | SH3KBP1 |
| ENSG00000174705 | SH3PXD2B |
| ENSG00000172985 | SH3RF3 |
| ENSG00000160691 | SHC1 |
| ENSG00000168995 | SIGLEC7 |
| ENSG00000138083 | SIX3 |
| ENSG00000155926 | SLA |
| ENSG00000109171 | SLAIN2 |
| ENSG00000117090 | SLAMF1 |
| ENSG00000026751 | SLAMF7 |
| ENSG00000007216 | SLC13A2 |
| ENSG00000117479 | SLC19A2 |
| ENSG00000115902 | SLC1A4 |
| ENSG00000168575 | SLC20A2 |
| ENSG00000175003 | SLC22A1 |
| ENSG00000163393 | SLC22A15 |
| ENSG00000085491 | SLC25A24 |
| ENSG00000155850 | SLC26A2 |
| ENSG00000091137 | SLC26A4 |
| ENSG00000225697 | SLC26A6 |
| ENSG00000083807 | SLC27A5 |
| ENSG00000117394 | SLC2A1 |
| ENSG00000014824 | SLC30A9 |
| ENSG00000198569 | SLC34A3 |
| ENSG00000110660 | SLC35F2 |
| ENSG00000141424 | SLC39A6 |
| ENSG00000138821 | SLC39A8 |
| ENSG00000137968 | SLC44A5 |
| ENSG00000004939 | SLC4A1 |
| ENSG00000256870 | SLC5A8 |
| ENSG00000163817 | SLC6A20 |
| ENSG00000092068 | SLC7A8 |
| ENSG00000066230 | SLC9A3 |
| ENSG00000184347 | SLIT3 |
| ENSG00000165300 | SLITRK5 |
| ENSG00000175387 | SMAD2 |
| ENSG00000120693 | SMAD9 |
| ENSG00000153147 | SMARCA5 |
| ENSG00000163029 | SMC6 |
| ENSG00000157106 | SMG1 |
| ENSG00000235169 | SMIM1 |
| ENSG00000259120 | SMIM6 |
| ENSG00000145335 | SNCA |
| ENSG00000206755 | SNORA30 |
| ENSG00000239149 | SNORA59A |
| ENSG00000200478 | SNORD115-41 |
| ENSG00000201143 | SNORD115-42 |
| ENSG00000202261 | SNORD115-44 |
| ENSG00000163788 | SNRK |
| ENSG00000167208 | SNX20 |
| ENSG00000112335 | SNX3 |
| ENSG00000162627 | SNX7 |
| ENSG00000120833 | SOCS2 |
| ENSG00000180008 | SOCS4 |
| ENSG00000112096 | SOD2 |
| ENSG00000154556 | SORBS2 |
| ENSG00000108018 | SORCS1 |
| ENSG00000079263 | SP140 |
| ENSG00000076382 | SPAG5 |
| ENSG00000133104 | SPG20 |
| ENSG00000197912 | SPG7 |
| ENSG00000116096 | SPR |
| ENSG00000167778 | SPRYD3 |
| ENSG00000123178 | SPRYD7 |
| ENSG00000115306 | SPTBN1 |
| ENSG00000122862 | SRGN |
| ENSG00000075142 | SRI |
| ENSG00000140319 | SRP14 |
| ENSG00000167881 | SRP68 |
| ENSG00000179954 | SSC5D |
| ENSG00000141298 | SSH2 |
| ENSG00000197558 | SSPO |
| ENSG00000100380 | ST13 |
| ENSG00000126091 | ST3GAL3 |
| ENSG00000214188 | ST7-OT4 |
| ENSG00000185482 | STAC3 |
| ENSG00000115145 | STAM2 |
| ENSG00000147465 | STAR |
| ENSG00000126549 | STATH |
| ENSG00000123473 | STIL |
| ENSG00000112079 | STK38 |
| ENSG00000137868 | STRA6 |
| ENSG00000266173 | STRADA |
| ENSG00000242866 | STRC |
| ENSG00000166763 | STRCP1 |
| ENSG00000099365 | STX1B |
| ENSG00000103496 | STX4 |
| ENSG00000064607 | SUGP2 |
| ENSG00000177688 | SUMO4 |
| ENSG00000148291 | SURF2 |
| ENSG00000264538 | SUZ12P |
| ENSG00000185518 | SV2B |
| ENSG00000162520 | SYNC |
| ENSG00000129990 | SYT5 |
| ENSG00000147041 | SYTL5 |
| ENSG00000115353 | TACR1 |
| ENSG00000165632 | TAF3 |
| ENSG00000148835 | TAF5 |
| ENSG00000187325 | TAF9B |
| ENSG00000164691 | TAGAP |
| ENSG00000102125 | TAZ |
| ENSG00000175463 | TBC1D10C |
| ENSG00000105254 | TBCB |
| ENSG00000110719 | TCIRG1 |
| ENSG00000185339 | TCN2 |
| ENSG00000124678 | TCP11 |
| ENSG00000162782 | TDRD5 |
| ENSG00000099797 | TECR |
| ENSG00000120156 | TEK |
| ENSG00000149256 | TENM4 |
| ENSG00000159648 | TEPP |
| ENSG00000131126 | TEX101 |
| ENSG00000136478 | TEX2 |
| ENSG00000008196 | TFAP2B |
| ENSG00000116819 | TFAP2E |
| ENSG00000162851 | TFB2M |
| ENSG00000160182 | TFF1 |
| ENSG00000092295 | TGM1 |
| ENSG00000169231 | THBS3 |
| ENSG00000130775 | THEMIS2 |
| ENSG00000100296 | THOC5 |
| ENSG00000005108 | THSD7A |
| ENSG00000116001 | TIA1 |
| ENSG00000166548 | TK2 |
| ENSG00000101342 | TLDC2 |
| ENSG00000137462 | TLR2 |
| ENSG00000101916 | TLR8 |
| ENSG00000141524 | TMC6 |
| ENSG00000162542 | TMCO4 |
| ENSG00000170348 | TMED10 |
| ENSG00000086598 | TMED2 |
| ENSG00000139173 | TMEM117 |
| ENSG00000011638 | TMEM159 |
| ENSG00000146842 | TMEM209 |
| ENSG00000089063 | TMEM230 |
| ENSG00000155755 | TMEM237 |
| ENSG00000165152 | TMEM246 |
| ENSG00000106609 | TMEM248 |
| ENSG00000182107 | TMEM30B |
| ENSG00000151715 | TMEM45B |
| ENSG00000204178 | TMEM57 |
| ENSG00000116209 | TMEM59 |
| ENSG00000165548 | TMEM63C |
| ENSG00000133872 | TMEM66 |
| ENSG00000165071 | TMEM71 |
| ENSG00000167874 | TMEM88 |
| ENSG00000137103 | TMEM8B |
| ENSG00000175348 | TMEM9B |
| ENSG00000153802 | TMPRSS11D |
| ENSG00000232810 | TNF |
| ENSG00000185215 | TNFAIP2 |
| ENSG00000173535 | TNFRSF10C |
| ENSG00000141655 | TNFRSF11A |
| ENSG00000157873 | TNFRSF14 |
| ENSG00000127863 | TNFRSF19 |
| ENSG00000028137 | TNFRSF1B |
| ENSG00000215788 | TNFRSF25 |
| ENSG00000186827 | TNFRSF4 |
| ENSG00000049249 | TNFRSF9 |
| ENSG00000125735 | TNFSF14 |
| ENSG00000130595 | TNNT3 |
| ENSG00000173726 | TOMM20 |
| ENSG00000143337 | TOR1AIP1 |
| ENSG00000092203 | TOX4 |
| ENSG00000186815 | TPCN1 |
| ENSG00000162341 | TPCN2 |
| ENSG00000198467 | TPM2 |
| ENSG00000158109 | TPRG1L |
| ENSG00000056558 | TRAF1 |
| ENSG00000127191 | TRAF2 |
| ENSG00000009790 | TRAF3IP3 |
| ENSG00000211868 | TRAJ21 |
| ENSG00000211859 | TRAJ30 |
| ENSG00000211853 | TRAJ36 |
| ENSG00000211844 | TRAJ45 |
| ENSG00000211842 | TRAJ47 |
| ENSG00000211840 | TRAJ49 |
| ENSG00000115993 | TRAK2 |
| ENSG00000255569 | TRAV1-1 |
| ENSG00000211794 | TRAV12-3 |
| ENSG00000211818 | TRAV39 |
| ENSG00000211804 | TRDV1 |
| ENSG00000072657 | TRHDE |
| ENSG00000204616 | TRIM31 |
| ENSG00000231226 | TRIM31-AS1 |
| ENSG00000134253 | TRIM45 |
| ENSG00000147573 | TRIM55 |
| ENSG00000100505 | TRIM9 |
| ENSG00000188917 | TRMT2B |
| ENSG00000100991 | TRPC4AP |
| ENSG00000167723 | TRPV3 |
| ENSG00000182612 | TSPAN10 |
| ENSG00000168785 | TSPAN5 |
| ENSG00000158526 | TSR2 |
| ENSG00000178952 | TUFM |
| ENSG00000140830 | TXNL4B |
| ENSG00000011600 | TYROBP |
| ENSG00000272173 | U47924.31 |
| ENSG00000182179 | UBA7 |
| ENSG00000154127 | UBASH3B |
| ENSG00000078967 | UBE2D4 |
| ENSG00000170035 | UBE2E3 |
| ENSG00000009335 | UBE3C |
| ENSG00000135018 | UBQLN1 |
| ENSG00000188021 | UBQLN2 |
| ENSG00000104517 | UBR5 |
| ENSG00000154277 | UCHL1 |
| ENSG00000198276 | UCKL1 |
| ENSG00000109814 | UGDH |
| ENSG00000242515 | UGT1A10 |
| ENSG00000156096 | UGT2B4 |
| ENSG00000174607 | UGT8 |
| ENSG00000059145 | UNKL |
| ENSG00000243566 | UPK3B |
| ENSG00000188690 | UROS |
| ENSG00000006611 | USH1C |
| ENSG00000162402 | USP24 |
| ENSG00000101558 | VAPA |
| ENSG00000071246 | VASH1 |
| ENSG00000197415 | VEPH1 |
| ENSG00000206538 | VGLL3 |
| ENSG00000151445 | VIPAS39 |
| ENSG00000154978 | VOPP1 |
| ENSG00000163032 | VSNL1 |
| ENSG00000132821 | VSTM2L |
| ENSG00000167992 | VWCE |
| ENSG00000176473 | WDR25 |
| ENSG00000163811 | WDR43 |
| ENSG00000085433 | WDR47 |
| ENSG00000166415 | WDR72 |
| ENSG00000103175 | WFDC1 |
| ENSG00000115935 | WIPF1 |
| ENSG00000116729 | WLS |
| ENSG00000165238 | WNK2 |
| ENSG00000002745 | WNT16 |
| ENSG00000124343 | XG |
| ENSG00000171044 | XKR6 |
| ENSG00000182489 | XKRX |
| ENSG00000143324 | XPR1 |
| ENSG00000196419 | XRCC6 |
| ENSG00000006047 | YBX2 |
| ENSG00000163872 | YEATS2 |
| ENSG00000177311 | ZBTB38 |
| ENSG00000104219 | ZDHHC2 |
| ENSG00000165861 | ZFYVE1 |
| ENSG00000155256 | ZFYVE27 |
| ENSG00000141497 | ZMYND15 |
| ENSG00000123870 | ZNF137P |
| ENSG00000179909 | ZNF154 |
| ENSG00000010539 | ZNF200 |
| ENSG00000159917 | ZNF235 |
| ENSG00000145908 | ZNF300 |
| ENSG00000175213 | ZNF408 |
| ENSG00000196724 | ZNF418 |
| ENSG00000183621 | ZNF438 |
| ENSG00000142528 | ZNF473 |
| ENSG00000152433 | ZNF547 |
| ENSG00000251369 | ZNF550 |
| ENSG00000171970 | ZNF57 |
| ENSG00000180357 | ZNF609 |
| ENSG00000167528 | ZNF641 |
| ENSG00000179930 | ZNF648 |
| ENSG00000251192 | ZNF674 |
| ENSG00000120963 | ZNF706 |
| ENSG00000140548 | ZNF710 |
| ENSG00000133624 | ZNF767 |
| ENSG00000224689 | ZNF812 |
| ENSG00000151612 | ZNF827 |
| ENSG00000221923 | ZNF880 |
| ENSG00000180532 | ZSCAN4 |

Evaluation of the Validation Performance and Other Statistical Analysis

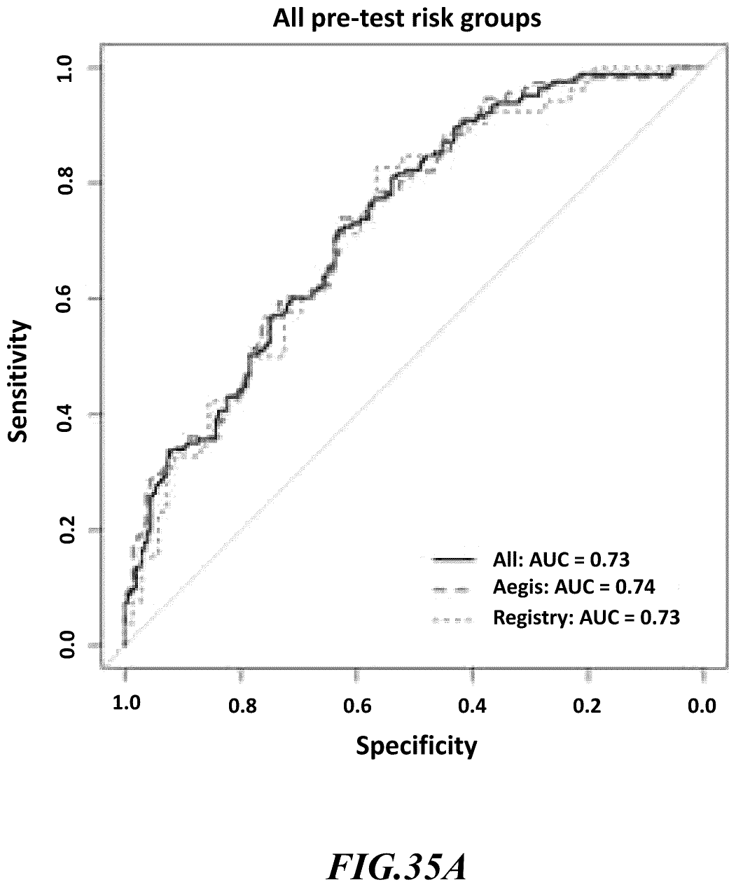

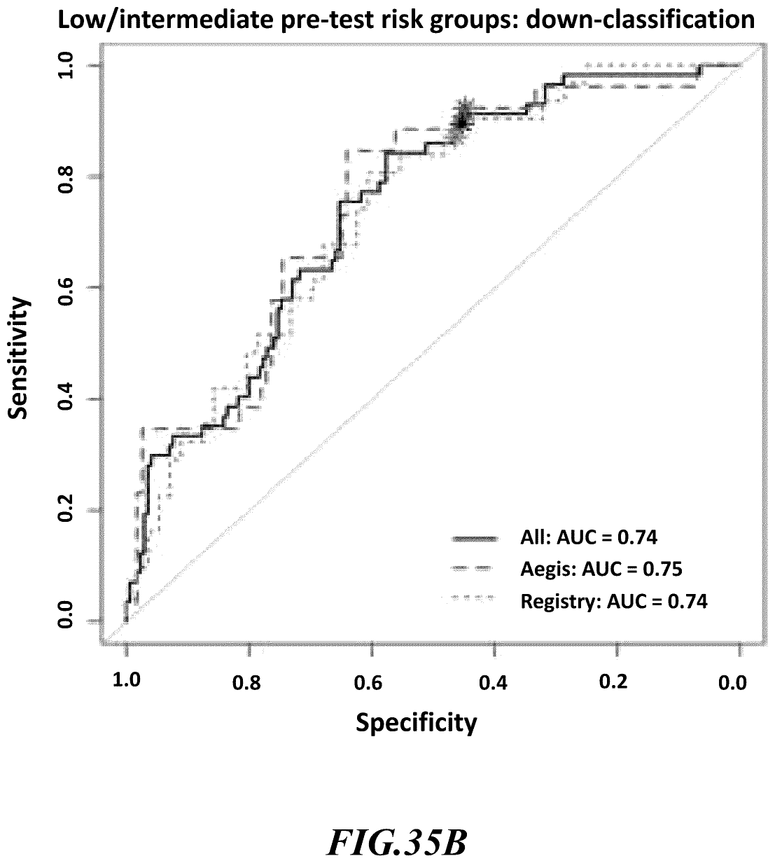

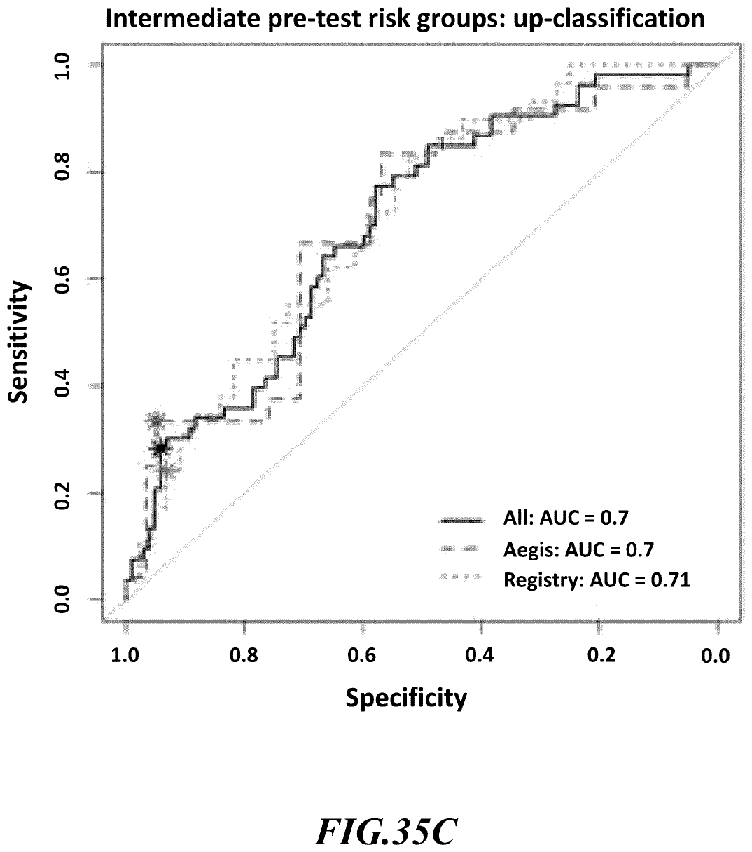

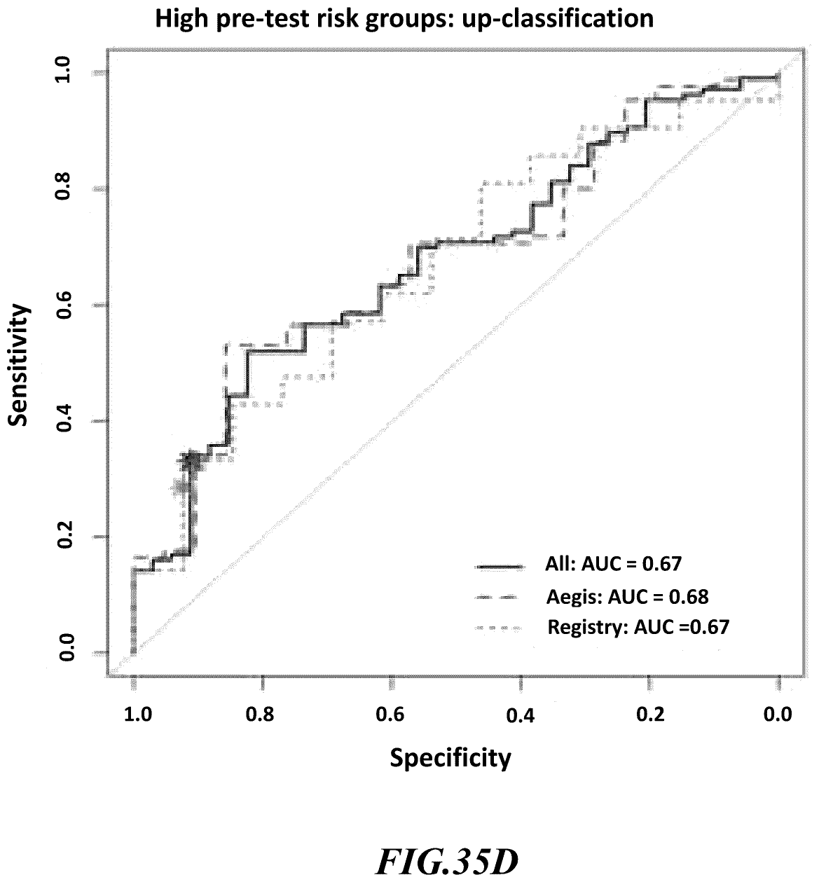

This independent validation set included 412 patients with nodules either low, intermediate or high pre-test ROM. The cancer prevalence together with GSC's sensitivity and specificity were used for the computation of negative predicted value (NPV) when down-classifying the patient's cancer risk and positive predictive value (PPV) when up-classifying the patient's cancer risk. Descriptive statistics are reported for clinical demographic data by cohorts included in the final validation set. Significance of difference among cohorts was tested with the chi-square test for categorical variables and Wilcoxon rank test for continuous variables. All confidence intervals are two-sided 95% unless otherwise noted. Statistical analyses were performed in R (version 3.2.3, r-project.org). Performance of the classifier was also assessed without fixed thresholds utilizing a receiver operating curve (ROC) and calculation of the area under the curve (AUC). The ROC provided a comprehensive evaluation of the GSC classifier performance independent of the cut-offs across all three cohorts and in different pre-test ROM groups. (Table 34 and FIG. 35A-35D).

TABLE 34
GSC performance in patients in subset of patients with and without COPD

| | | COPD | | | non-COPD | | |
|---|---|---|---|---|---|---|---|
| Pre-test Cancer Risk | GSC result | N | Specificity | Sensitivity | N | Specificity | Sensitivity |
| Low | Very Low | 18 | 35.3% | 100% | 54 | 64.7% | 100% |
| | | | (14.2-61.7) | (2.5-100) | | (50.1-77.6) | (29.2-100) |
| Intermediate | Low | 54 | 18.2% | 95.2% | 101 | 46.4% | 87.5% |
| | | | (7.0-35.5) | (76.2-99.9) | | (34.3-58.8) | (71-96.5) |
| | High | | 90.9% | 47.6% | | 95.7% | 15.6% |
| | | | (75.7-98.1) | (25.7-70.2) | | (87.8-99.1) | (5.3-32.8) |
| High | Very High | 64 | 88.9% | 45.5% | 76 | 92.0% | 21.6% |
| | | | (51.8-99.7) | (32.0-59.4) | | (74.0-99.0) | (11.3-35.3) |

N, number of patients;
COPD, chronic obstructive pulmonary disease

Results

Clinical Study Population and Nodule Characteristics

Four hundred twelve patients from the AEGIS cohorts (I and II) (246 patients) and the Registry (166 patients) were included in the validation cohort for the GSC (Table 33 and FIGS. 33A and 33B) The most common histological types of cancer were adenocarcinoma (51%) followed by squamous cell (22%) lung cancer.

TABLE 33
Demographic and Clinical Characteristics of the Study Participants

| | AEGIS | | Registry | | Total | | |
|---|---|---|---|---|---|---|---|
| Characteristic | (N = 246) | | (N = 166) | | (N = 432) | | P-value |
| Sex | | | | | | | 0.001 |
| Female | 83 | ( [symbol] %) | 84 | (51%) | 157 | (40%) | |
| Male | 163 | ( [symbol] %) | 82 | (49%) | 245 | (59%) | |
| Median age (IQR) | 62 | ( [symbol] ) | [symbol] | ( [symbol] -71) | 63 | ( [symbol] -71) | 0.08 |
| Race | | | | | | | 0.38 |
| White | 192 | ( [symbol] %) | 132 | (80%) | [symbol] | ( [symbol] %) | |
| Black | 42 | ( [symbol] %) | 29 | (17%) | [symbol] | ( [symbol] %) | |
| Other | 12 | ( [symbol] %) | 4 | ( [symbol] %) | [symbol] | ( [symbol] %) | |
| Unknown | 0 | | 1 | ( [symbol] %) | 1 | (0.2%) | |
| Smoking status | | | | | | | 0.92 |
| Current | 107 | ( [symbol] %) | 73 | (44%) | 180 | ( [symbol] %) | |
| Former | [symbol] | ( [symbol] %) | 93 | (56%) | 232 | ( [symbol] %) | |
| Median cumulative tobacco use (IQR) | 35 | ( [symbol] ) | [symbol] | ( [symbol] ) | 35 | ( [symbol] ) | 0.89 |
| -pack-year | | | | | | | |
| L [symbol] size | | | | | | | <0.001 |
| Infiltrate* | 25 | ( [symbol] ) | 0 | | 25 | (6%) | |
| <2 cm | [symbol] | ( [symbol] %) | 80 | (48%) | [symbol] | (41%) | |
| 2 to 3 cm | [symbol] | (20%) | 29 | ( [symbol] %) | 77 | (19%) | |
| >3 cm | [symbol] | (30% | 44 | ( [symbol] %) | 119 | ( [symbol] %) | |
| Unknown | [symbol] | (4%) | 13 | ( [symbol] %) | 25 | (6%) | |
| Lesion location | | | | | | | <0.001 |
| Central | 72 | ( [symbol] %) | 10 | (6%) | 82 | ( [symbol] %) | |
| Peripheral | 108 | (44%) | 144 | ( [symbol] %) | [symbol] | (61%) | |
| Central and peripheral | [symbol] | ( [symbol] %) | 0 | | 53 | (35%) | |
| Unknown | 13 | (5%) | 12 | (7%) | [symbol] | ( [symbol] %) | |
| Lung-cancer histologic type | 111 | (45%) | 52 | (31%) | 163 | (40%) | 0.01 |
| Small cell lung cancer | [symbol] | (7%) | 1 | (2%) | 9 | (6%) | |
| Non-small cell lung cancer | 100 | (90%) | 43 | ( [symbol] %) | 145 | ( [symbol] %) | 0.43 |
| Adenocarcinoma | [symbol] | (58%) | [symbol] | ( [symbol] %) | 83 | (58%) | |
| Squamous | 26 | ( [symbol] %) | [symbol] | ( [symbol] %) | [symbol] | ( [symbol] %) | |
| Large-cell | 4 | (4%) | 0 | | 4 | (3%) | |
| NSCLC-NOS | [symbol] | ( [symbol] %) | 8 | (19%) | 20 | (14%) | |
| Carcinoid | 0 | | 2 | (4%) | [symbol] | (3%) | |
| Unknown | 3 | (3%) | 6 | (12%) | 9 | (6%) | |
| Diagnosis of a benign condition | 135 | (55%) | 114 | (69%) | 249 | (60%) | <0.001 |
| [symbol] | 26 | (19%) | [symbol] | ( [symbol] %) | 36 | ( [symbol] %) | |
| Infection | 36 | (27%) | 35 | ( [symbol] %) | 51 | ( [symbol] %) | |
| Two or more benign conditions | 3 | (6%) | 0 | | [symbol] | ( [symbol] %) | |
| Other | 27 | ( [symbol] %) | 4 | ( [symbol] ) | 31 | ( [symbol] %) | |
| Resolution of Stability | [symbol] | (28%) | 40 | (35%) | [symbol] | ( [symbol] %) | |

| | | | | | |
|---|---|---|---|---|---|
| Clinically benign** | 0 | [symbol] | (39%) | 45 | ( [symbol] %) |

IQR, intraquartile range;
NSCLC-NOS, non-small cell lung cancer- not otherwise specified
Percentages are calculated within each study cohort, i.e. AEGIS, and the [symbol] Registry, respectively; for sub-level breakdowns, i.e. cancer histologic subtype and benign condition, the [symbol] is the sub-group count
*Infiltrates are pulmonary [symbol] with ill-defined margins and 2 diameter that cannot be accurately defined.
**Clinically benign did not have an adjudicated diagnosis but were included in the analysis for cancer prevalence to prevent an over-estimate. [symbol]
indicates data missing or illegible when filed

Performance of GSC in Indeterminate Nodules Stratified by Risk of Malignancy

Approximately 19% of the cohort was defined as low risk (cancer prevalence of 5.0%), 46% were defined as intermediate risk (cancer prevalence of 28.2%) and 35% were defined as high risk (cancer prevalence of 74.0%). Intermediate-risk nodules were down-classified to low risk with a sensitivity of 90.6% and a specificity of 37.3%. With a 28.2% cancer prevalence, 29.4% of intermediate-risk nodules were down-classified with a 91.0% (Confidence Interval (CI), 80.8-96.0) NPV. Intermediate-risk nodules were up-classified to high risk with a 94.1% specificity and 28.3% sensitivity. With a 28.2% cancer prevalence, 12.2% of intermediate risk nodules were up-classified with a 65.4% (CI, 43.8-82.1) positive predictive value (PPV). Low-risk nodules were further down-classified to very low risk in 54.5% of tests with a 100% sensitivity indicating there are no false negatives and >99% negative predictive value (NPV) (CI, 91.0-100). High-risk nodules were up-classified to very high risk, with a specificity of 91.2% and a sensitivity of 34.0%. With a 73.6% cancer prevalence, 27.3% of high-risk nodules were up-classified with a 91.5% (CI, 77.9-97.0) PPV (Table 36).

TABLE 36
GSC performance.

| Pre-test | | | | | | | | % |
|---|---|---|---|---|---|---|---|---|
| Risk of | | | | | | | | Reclassified |
| Malignancy | | | Clinical | | | Percepta | Post-test | risk of |
| (cancerprevalence) | Malignant | Benign | Benign | Specificity | Sensitivity | GSC result | NPV/PPV | malignancy |
| Low | 4 | 68 | 8 | 57.4% | 100% | Very Low | 100% NPV | 54.5% |
| N = 80 | | | | (44.8- 69.3) | (39.8-100) | | (91.0-100) | |
| (5.0%) | | | | | | | | |
| Intermediate | 53 | 102 | 33 | 37.3% | 90.6% | Low | 91.0% NPV | 29.4% |
| N = 188 | | | | (27.9 - 47.4) | (79.3 - 96.9) | | (80.8 - 96.0) | |
| (28.2%) | | | | 94.1% | 28.3% | High | 65.4% PPV | 12.20% |
| | | | | (87.6- 97.8) | (16.8-42.3) | | (43.8 - 82.3) | |
| High | 156 | 34 | 4 | 91.2% | 34.0% | Very High | 93.5% PPV | 27.3% |
| N =144 | | | | (76.3- 98.3) | (25.0 - 43.8) | | (77.9 -97.0) | |
| (73.6%) | | | | | | | | |

N. number of patients; including malignant, benign and clinical benign patients
Cancer Prevalence is the proportion of malignant patients over total patients (N) including clinical benign.+
Specificity is calculated on benign patients only, excluding clinical benien; sensitivity is calculated on malignant patients only
PPV = Prevalence · Sensitivity/Prevalence · Sensitivity + (1-Prevalence) · (1-Specificity);
NPV = (1-Prevalence) · Specificity/Prevalence · (1-Sensitivity) + (1-Prevalence) · Specificity
% Reclassified (Low to Very Low, Intermediate to Low) = (1-Prevalence) specificity + Prevalence (1-sensitivity)
% Reclassified (Intermediate to High, High to Very High) = Prevalence · sensitivity + (1-Prevalence) (1-specificity)
NPV (negative predictive value, PPV (positive predictive value), and % Reclassified are all functions of sensitivity, specificity and cancer prevalence.

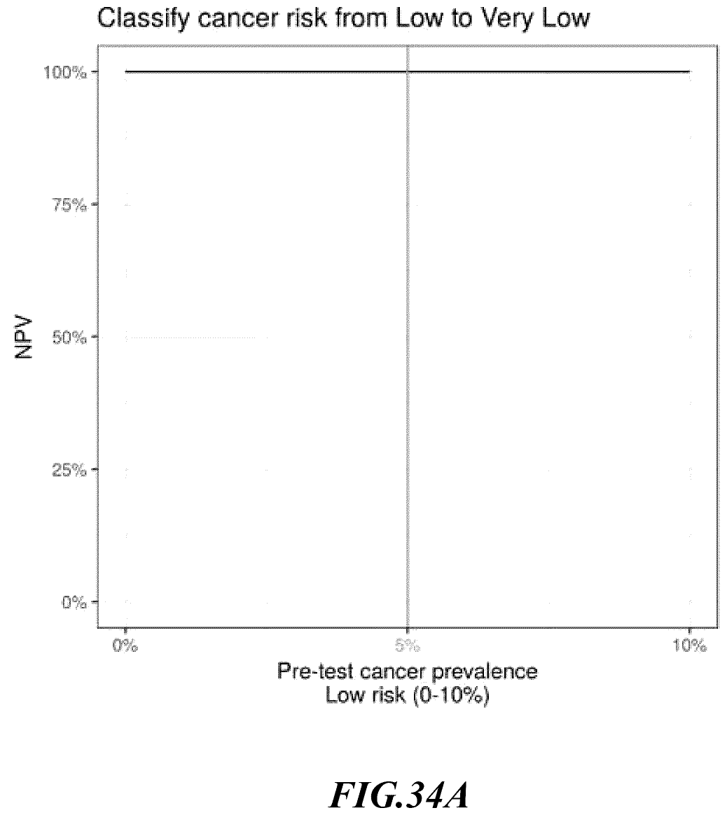

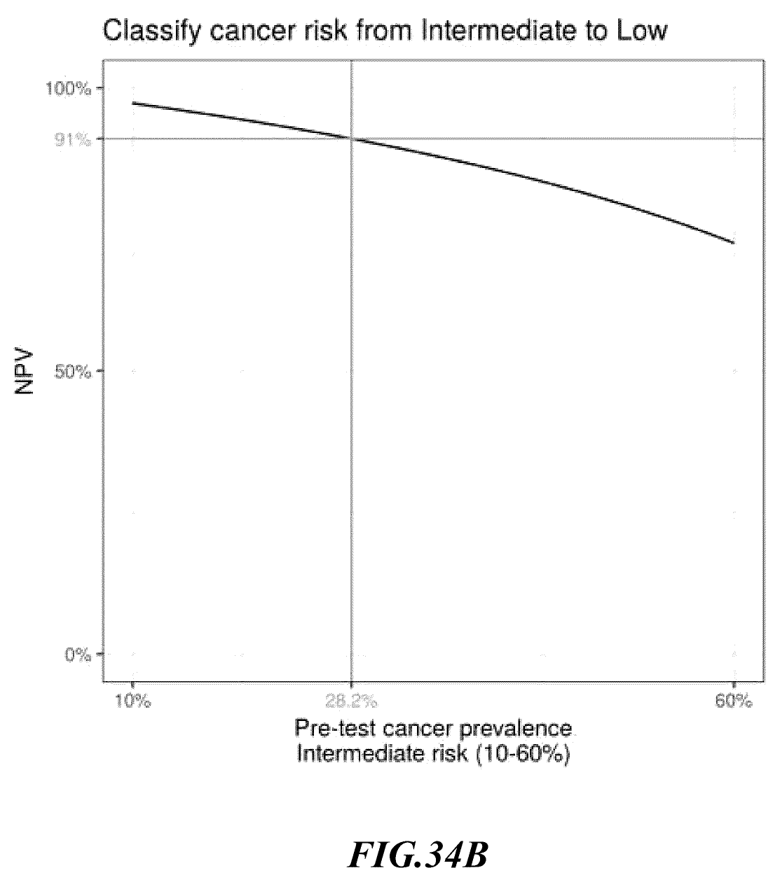

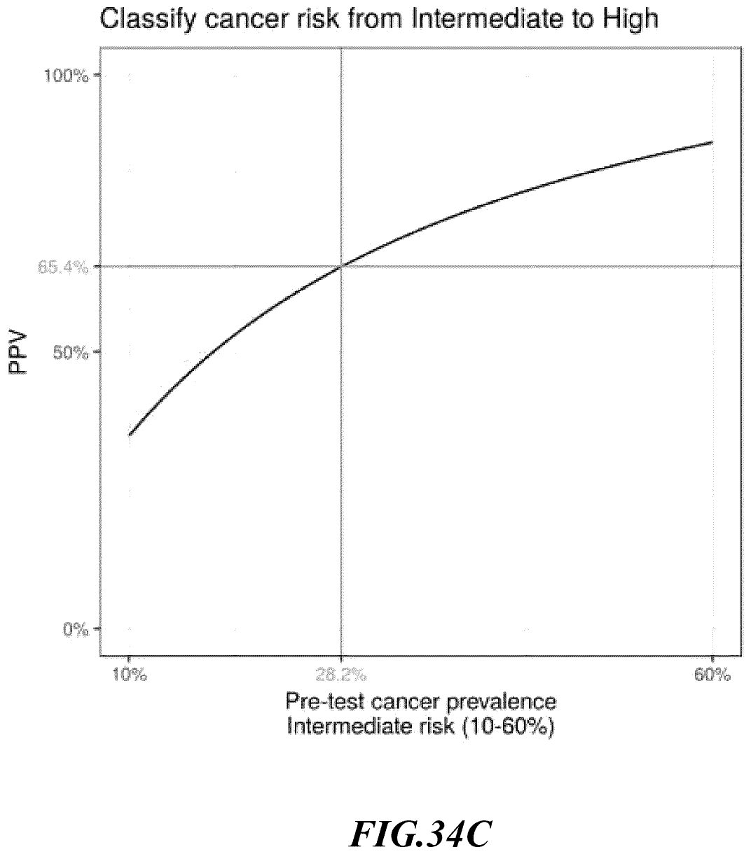

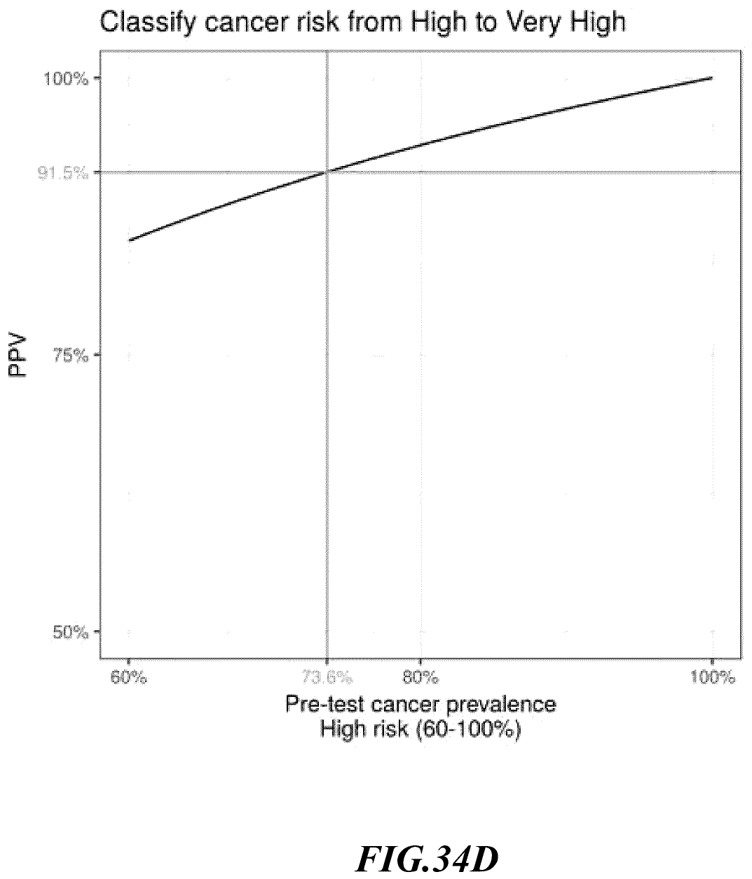

Among nodules that were up-classified from intermediate to high ROM, six nodules were benign. These false positives account for 6/102 (5.90%) of all benign intermediate-risk nodules. Among nodules that were down-classified from intermediate to low ROM, five nodules were malignant. These false negatives account for 5/53 (9.40%) of all malignant intermediate risk nodules. Among nodules that were up-classified from high to very high ROM, three nodules were benign. These false positives account for 3/34 (8.8%) of all benign high-risk nodules. There were no nodules that were falsely down classified from low to very low ROM. NPV and PPV estimates across a range of cancer prevalence are shown in FIG. 34A-34D.

We evaluated the accuracy of the GSC in patients with and without COPD. The sensitivity in those with COPD was slightly higher and the specificity slightly lower than those without COPD (Table 34).

We compared the overall performance of the Percepta GSC using a Receiver Operating Curve (ROC) to provide a comprehensive evaluation of the classifier performance independent of the cut-offs in all three cohorts. We found that the overall performance of the Percepta GSC was similar in the AEGIS I and II cohorts compared to the Percepta Registry with an overall Area Under the Curve (AUC) of 0.73 (CI. 68.3-78.4) highlighting the robustness of the classifier performance across different patient cohorts (Table 33, Table 35 and FIG. 35A-35D).

TABLE 35
GSC performance in patients in AEGIS I and II and Registry Cohorts

| Pre-test | AEGIS I and II | | | Registry | | |
|---|---|---|---|---|---|---|
| Cancer Risk | N | Specificity | Sensitivity | N | Specificity | Sensitivity |
| Low | 58 | 55.4% | 100% | 14 | 100% | 100% |
| | | (41.5-68.7) | (15.8-100) | | (2.5-100) | (2.5-100) |
| Intermediate | 82 | 34.5% | 91.7% | 73 | 40.9% | 89.7% |
| | | (22.5-48.1) | (73.0-99.0) | | (26.3-56.8) | (72.6-97.8) |
| | | 94.8% | 33.3% | | 93.2% | 24.1% |
| | | (85.6-98.9) | (15.6-55.3) | | (81.3-98.6) | (10.3-43.5) |
| High | 106 | 90.5% | 34.1% | 34 | 92.3% | 33.3% |
| | | (69.6-98.8) | (24.2-45.2) | | (64.0-99.8) | (14.6-57.0) |

AEGIS, Airway Epithelium Gene Expression In the Diagnosis of Lung Cancer,
N, number of patients

In this clinical validation study of the second generation lung nodule classifier, GSC, the accuracy of the classifier was validated in an independent sample set. A high sensitivity with modest specificity for the rule out portion of the classifier and high specificity with modest sensitivity for the rule in portion was confirmed. By accurately down-classifying and up-classifying a portion of those with indeterminate lung nodules and a nondiagnostic bronchoscopy, the classifier may influence later management decisions to the benefit of the patients.

As designed, when down-classifying the risk of malignancy (ROM), the classifier has high sensitivity and modest specificity. Thus, a negative result would lead to a reduced ROM, and a positive result confirms the pre-test risk assessment and management decisions. Similarly, when up-classifying the ROM, the classifier has a high specificity and modest sensitivity. Thus a positive result would lead to an increased ROM, and a negative result would confirm pre-test risk assessment and management decisions. Therefore, a portion of those tested will have a test result that could change pre-test clinical management decisions and a portion will confirm the pre-test management approach.

For those patients with an intermediate pre-test risk lung nodule and a non-diagnostic bronchoscopy, the classifier may be used to down-classify the risk, making the clinician more comfortable with surveillance of the nodule, or to up-classify the risk, suggesting additional testing or treatment is warranted. In the population studied within this risk group, the sensitivity of 90.6% and specificity of 37.3% for the down-classifier led to an actionable negative result in 29.4% of those tested with a ratio of true negative to false-negative results of 10:1. Thus if the test result led to surveillance imaging, 10 patients with benign nodules may have avoided further testing while 1 patient with a malignant nodule may have had further evaluation delayed. In the population studied within this risk group, the sensitivity of 28.3% and specificity of 94.1% for the up-classifier led to an actionable positive result in 12.2% of those tested with a ratio of true positive to false-positive results of 1.9:1. Thus if the test result led to more aggressive testing or treatment, approximately 2 patients with malignant nodules would proceed to additional invasive testing or treatment while 1 patient with a benign nodule would do the same. Overall, 41.6% of patients with intermediate risk nodules and non-diagnostic bronchoscopies were classified to a lower or higher risk group. Additional studies will directly answer how often test results change management decisions, as these decisions are heavily influenced by local treatment patterns as well as patient values and comorbidities.

Similarly, the ability to risk stratify nodules with low and high pre-test probability of malignancy may lead to greater clinician or patient confidence with management choices. The test characteristics suggest that a negative result from the rule-out classifier may downgrade the risk of a patient with a low probability nodule and a positive result from the rule-in classifier may upgrade the risk of a patient with a high probability nodule. In the population studied, 54.5% of low-risk nodules were down-classified to very low risk without any false negatives reported, while 27.3% of high-risk nodules were up-classified to very high risk with a ratio of true positives to false positives of 12:1. Thus if the test result resulted in further aggressive therapy, approximately 12 patients with a malignant nodule would be referred for an additional invasive procedure, whereas 1 patient with a benign nodule would also undergo the same. When the classifier is used across categories of risk (low, intermediate, and high) 39.1% of tests would classify the patient to a category of risk that is different from their pre-test risk category.

The comparison results of test accuracy between those with and without COPD provides interesting insight into the nature of the classifier and the field of injury concept. In general, the classifier had a higher sensitivity and lower specificity in those with COPD whether used as a rule-in or rule-out test. This may suggest some signature overlap between genomic changes and clinical features with COPD and lung cancer, such that some positive results are identifying shared features between the two conditions, perhaps reflecting the increased risk of lung cancer in the COPD population. This knowledge may further increase confidence in negative results in a COPD patient and positive results in those without COPD.

Strengths of the study include three large, heterogeneous, independent cohorts to assess clinical accuracy metrics of the GSC, locked-down after completion of algorithm development and technical validation phases. The updated classifier extends the range of potential utility by adding a rule-in component to the test for patients with a pre-test intermediate-risk lung nodule. This clinical validation of the GSC was performed in patients with a non-diagnostic bronchoscopy, reflecting the accuracy where the test will have potential utility.

Limitations of the results include the adjudication process where follow-up was only required to be 12 months to determine benign status. This may have contributed to the inability to adjudicate 45 samples (not included in the sensitivity and specificity metrics but used to estimate prevalence assuming benignity). Thus a few indolent lung cancers could have been present and the true prevalence of malignancy may have been slightly higher. It is unclear whether identifying indolent malignancies would impact the utility of the classifier, as surveillance of indolent malignancies is less likely to influence outcomes.

As is true with all risk of malignancy prediction models, shifts from one risk category to another are based on negative and positive predictive values, the calculation of which requires the prevalence of malignancy within those risk groups. This study utilized three independent cohorts to establish cancer prevalence at each risk level, however, prevalence may vary in an individual clinical practice. To assist with the application of the test, we provided figures showing post-test probabilities across a range of pre-test probabilities in the supplement, assuming consistent sensitivity and specificity across all pre-test ROMs (FIG. 35A-35D).

This clinical validation study confirmed the accuracy of the GSC, showing high sensitivity for the rule-out portion of the classifier and high specificity for the rule-in portion of the classifier. Use of the classifier could impact clinical decisions in up to 40% of patients with lung nodules and indeterminate results from bronchoscopy. Further assessment of clinical utility is warranted.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-101. (canceled)

102. A method, comprising:
(a) upon obtaining a first level of risk of malignancy of a subject for having or developing a cancer, obtaining a data set corresponding to a sample of said subject;
(b) in a programmed computer, using a classifier to assign said data set corresponding to said sample a second level of risk of malignancy for having or developing said cancer; and
(c) electronically outputting a report comprising said second level of risk of malignancy of (b) assigned to said sample of said subject,
wherein said second level of risk of malignancy is determined with a negative predictive value greater than 90%.

103. The method of claim 102, wherein said first level of risk of malignancy is 10% to 60% and said second level of risk of malignancy is greater than 60% or less than 10%.

104. The method of claim 102, wherein said data set comprises one or more genomic features.

105. The method of claim 104, wherein said one or more genomic features comprise a genomic smoking status or genomic gender.

106. The method of claim 104, wherein said one or more genomic features comprise gene expression products of genes differentially expressed in subjects that have said cancer and subjects that do not have said cancer.

107. The method of claim 102, wherein said cancer is a lung cancer.

108. The method of claim 102, wherein said first level of risk of malignancy is obtained based at least on a physical examination of the subject.

109. The method of claim 108, wherein said physical examination comprises a computed tomography scan, a non-surgical biopsy, a diagnostic bronchoscopy, or a combination thereof.

110. The method of claim 102, wherein said first level of risk of malignancy is inconclusive for said cancer.

111. The method of claim 102, wherein said data set comprises one or more clinical features.

112. The method of claim 111, wherein said one or more clinical features are selected from the group consisting of: age, gender, smoking status, number of years since subject quit smoking, length of a nodule, infiltrate nodule of the subject, and any combination thereof.

113. The method of claim 102, wherein said data set comprises one or more gene expression products.

114. The method of claim 113, wherein said gene expression products correspond to one or more genes set forth in Table 37, or a derivative thereof.

115. The method of claim 102, wherein said classification in (b) comprises applying a trained algorithm to said data set to determine the second level of risk of malignancy for having or developing said cancer, and wherein the trained algorithm is trained with a training data set.

116. The method of claim 115, wherein said training data set comprises sequence information derived from transcripts of bronchial or nasal epithelial cells.

117. The method of claim 115, wherein said training data set comprises data from samples of current smokers and former smokers.

118. The method of claim 115, wherein said training data set comprises data from (i) samples obtained from subjects that have a high risk, (ii) samples obtained from subjects that have an intermediate risk, or (iii) samples obtained from subjects that have a low risk of malignancy, based on diagnostic bronchoscopy.

119. The method of claim 115, wherein said training data set comprises data from samples obtained from subjects that have lung nodules that are inconclusive for lung cancer as determined by computed tomography scan or bronchoscopy.

120. The method of claim 102, further comprising obtaining said sample from said subject by collecting nasal epithelial cells from a nasal passage of said subject or collecting bronchial epithelial cells by bronchial brushing.

121. The method of claim 102, wherein said first level of risk of malignancy is based upon identification of nodule(s) or lesion(s) from a CT scan.

122. The method of claim 102, wherein said second level of risk of malignancy is less than 10% and wherein said classifier assigns said second level of risk of malignancy with a negative predictive value (NPV) of 95% or higher.

123. The method of claim 102, wherein said second level of risk of malignancy is greater than 60% and wherein said classifier assigns said second level of risk of malignancy with a positive predictive value (PPV) of 65% or greater.
